(19) United States
(12) Patent Application Publication
(10) Pub. No.: US 2015/0368242 A1
(43) Pub. Date: Dec. 24, 2015

(54) NOVEL TETRAHYDROPYRIDOPYRIMIDINONE DERIVATIVE

(71) Applicant: Ajinomoto Co., Inc., Chuo-ku (JP)

(72) Inventors: Tamotsu Suzuki, Kawasaki-shi (JP); Hiroki Inoue, Chuo-ku (JP); Kayo Matsumoto, Kawasaki-shi (JP); Takahiro Koshiba, Yokkaichi-shi (JP); Koji Ohsumi, Kawasaki-shi (JP); Hiroki Ozawa, Chuo-ku (JP); Munetaka Tokumasu, Kawasaki-shi (JP); Masatsugu Noguchi, Kawasaki-shi (JP)

(73) Assignee: Ajinomoto Co., Inc., Chuo-ku (JP)

(21) Appl. No.: 14/838,419

(22) Filed: Aug. 28, 2015

Related U.S. Application Data
(63) Continuation of application No. PCT/JP2014/055065, filed on Feb. 28, 2014.

(30) Foreign Application Priority Data
Feb. 28, 2013 (JP) 2013-039540

(51) Int. Cl. C07D 471/04

(52) U.S. Cl. CPC C07D 471/04; C07B 59/002

(57) ABSTRACT

An object is to provide a novel compound having a higher MGAT2 inhibitory activity than conventional compounds. A compound represented by the following general formula (I) or a pharmaceutically acceptable salt thereof is provided.

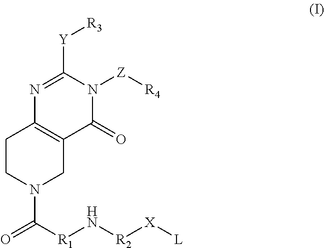

### CROSS-REFERENCE TO RELATED APPLICATIONS

The present application is a continuation of International Application No. PCT/JP2014/055605, filed Feb. 28, 2014, which is based upon and claims the benefits of priority to Japanese Application No. 2013-039540, filed Feb. 28, 2013. The entire contents of these applications are incorporated herein by reference.

### TECHNICAL FIELD

The present invention relates to a novel tetrahydropyridopyrimidinone derivative having monoacylglycerol acyltransferase inhibitory activity, a physiologically acceptable salt thereof, and a pharmaceutical composition comprising the same.

### BACKGROUND ART

Various disorders including impaired glucose tolerance, type 2 diabetes mellitus, lipid metabolism disorders, hypertension, and the like are known as health problems caused by or associated with obesity, and it is important to treat adiposity for preventing or alleviating these diseases. Diet therapy, exercise therapy, and behavior therapy are employed for treating adiposity, and, if necessary, drug therapy is introduced. Currently, drugs such as orlistat, mazindol, and sibutramine have been used as anti-obesity agents. However, none of these drugs is sufficiently satistfactory in terms of both drug efficacy and side effects, and there is a need for development of a better agent.

Neutral fats (triacylglycerols (TGs)) from diet are hydrolyzed in the gastrointestinal tract with pancreatic lipase into 2-monoacylglycerols (MGs) and free fatty acids, which are then absorbed into intestinal mucosal epithelial cells in the small intestine, where TGs are re-synthesized. The re-synthesis reaction is known to proceed through the monoacylglycerol pathway catalysed by monoacylglycerol acyltransferase (MGAT) and diacylglycerol acyltransferase (DGAT), and through the glycerol 3-phosphate pathway. In the small intestine, 70 to 80% of postprandial TG re-synthesis is undertook by the monoacylglycerol pathway, and the re-synthesized TGs are incorporated into chylomicrons together with other lipids, and transported through the blood to the liver and tissues such as adipose tissues.

MGAT is an enzyme for acylation of MGs to diacylglycerols (DGs), and three isoforms, namely, MGAT1, MGAT2, and MGAT3, have been identified so far. Of these isoforms, MGAT2 and MGAT3, which are highly expressed in the small intestine, are assumed to take part in the TG re-synthesis in the small intestine.

It is reported that, in an experiment using mice, a high-fat diet load increases the expression of MGAT2 in the small intestine, and the MGAT activity is increased (Non Patent Literature 1). In addition, in MGAT2-deficient mice on high-fat diet, reduction in weight gain, reduction in induction of insulin resistance, suppression of increase in blood cholesterol level, suppression of formation of fatty liver and the like, and increase in energy consumption were observed, and MGAT2 is shown to be a key enzyme of lipid absorption and energy metabolism (Non Patent Literature 2).

From these findings, it is predicted that an MGAT2 enzyme activity inhibitor is useful for treating or preventing obesity and various diseases associated with obesity.

Conventional compounds having MGAT2 inhibitory activity include several compounds described in Patent Literatures 1 to 4. However, the MGAT2 inhibitory activity of each of these compounds is not very high, and there is a great demand for a novel compound which has a higher MGAT2 inhibitory activity, and, accordingly, which is more useful for inhibiting fat absorption or for treating or preventing adiposity than conventional compounds.

### CITATION LIST

### Patent Literatures

Patent Literature 1: International Publication No. WO2008/038768
Patent Literature 2: International Publication No. WO2010/095767
Patent Literature 3: International Publication No. WO2012/091010
Patent Literature 4: International Publication No. WO2012/124744

### Non Patent Literatures

Non Patent Literature 1: Journal of Biological Chemistry, 279, 18878-18886, 2004
Non Patent Literature 2: Nature Medicine, 15, 442-446, 2009

### SUMMARY OF INVENTION

### Technical Problem

The present invention has been made in view of the above-described problem, and an object of the present invention is to provide a novel compound having a high MGAT2 inhibitory activity.

### Solution to Problem

The present inventors have found that a compound represented by the following general formula (I) or a physiologically acceptable salt thereof has an extremely high MGAT2 inhibition action, and this finding has led to the completion of the present invention. Specifically, the present invention includes the following components.

<1> A compound represented by the following general formula (I) or a pharmaceutically acceptable salt thereof:

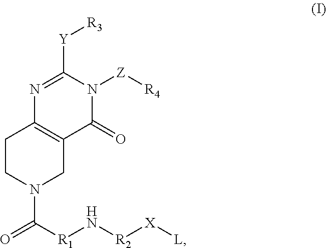

wherein

R₁ represents a linear C₁₋₆ alkylene group which may be optionally substituted by deuterium atoms;

R₂ represents a linear C₁₋₆ alkylene group or a cyclic C₃₋₆ alkylene group, which may be optionally substituted by 1 to 6 identical or different substituents selected from deuterium atoms, halogen atoms, C₁₋₃ alkyl groups, C₁₋₃ haloalkyl groups, C₁₋₃ alkylthio groups, nitrile groups, hydroxyl groups, C₁₋₃ alkoxy groups, C₁₋₃ haloalkoxy groups, amino groups, C₁₋₃ alkylamino groups, and C₂₋₆ dialkylamino groups;

X represents a single bond, —O—, —S—, or —NR₅—, where R₅ represents a hydrogen atom, a C₁₋₆ alkyl group, or a C₁₋₆ haloalkyl group;

L represents a C₂₋₆ alkenyl group, a C₂₋₆ alkynyl group, a cycloalkyl group having 3 to 6 carbon atoms, a phenyl group, a naphthyl group, a 3- to 6-membered saturated or unsaturated heterocyclic group, or a 9- or 10-membered saturated or unsaturated heterobicyclic group, which may be optionally substituted by 1 to 5 identical or different substituents selected from deuterium atoms, halogen atoms, hydroxyl groups, amino groups, C₁₋₃ alkyl groups, C₁₋₃ haloalkyl groups, C₁₋₃ alkylthio groups, nitrile groups, C₁₋₃ alkoxy groups, C₁₋₃ haloalkoxy groups, C₁₋₃ alkylamino groups, and C₂₋₆ dialkylamino groups, or which may be optionally monosubstituted by a substituent selected from the following (a) to (e):

(a) a —C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;

(b) a —C₁₋₃ alkylene-O—C₁₋₆ alkyl group which may be optionally substituted by 1 to 5 identical or different substituents selected from deuterium atoms, halogen atoms, C₁₋₃ alkoxy groups, and C₁₋₃ haloalkoxy groups;

(c) a —C₁₋₃ alkylene-O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;

(d) an —O—C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups; and

(e) an —O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;

Y represents a single bond, —S—, —O—, or —NR₆—, where R₆ represents a hydrogen atom, a C₁₋₆ alkyl group, or a C₁₋₆ haloalkyl group;

Z represents a single bond or a C₁₋₆ alkylene group;

R₃ represents a C₃₋₈ alkyl group, a C₃₋₈ cycloalkyl group, or a 3- to 8-membered saturated or unsaturated heterocyclic group, and may optionally have 1 to 7 identical or different substituents selected from the group consisting of deuterium atoms, halogen atoms, C₁₋₃ alkyl groups, C₁₋₃ haloalkoxy groups, and C₁₋₃ haloalkyl groups; and

R₄ represents a C₁₋₆ alkyl group, a phenyl group, a cycloalkyl group having 3 to 8 carbon atoms, or a 3- to 8-membered saturated or unsaturated heterocyclic group, and may optionally have 1 to 7 identical or different substituents selected from the group consisting of deuterium atoms, halogen atoms, C₁₋₃ alkyl groups, C₁₋₃ haloalkyl groups, C₁₋₃ alkoxy groups, and C₁₋₃ haloalkoxy groups.

<2> The compound according to the above-described <1> or a pharmaceutically acceptable salt thereof, wherein

X is a single bond, —O—, —S—, or —NH—.

<3> The compound according to the above-described <1> or <2>, or a pharmaceutically acceptable salt thereof, wherein

R₁ is a linear C₁₋₃ alkylene group.

<4> The compound according to any one of the above-described <1> to <3> or a pharmaceutically acceptable salt thereof, wherein

L is a phenyl group, a naphthyl group, a 5- or 6-membered saturated or unsaturated heterocyclic group, or a 9- or 10-membered saturated or unsaturated heterobicyclic group which may be optionally substituted by 1 to 5 identical or different substituents selected from deuterium atoms, halogen atoms, hydroxyl groups, amino groups, C₁₋₃ alkyl groups, C₁₋₃ haloalkyl groups, C₁₋₃ alkylthio groups, nitrile groups, C₁₋₃ alkoxy groups, C₁₋₃ haloalkoxy groups, C₁₋₃ alkylamino groups, and C₂₋₆ dialkylamino groups, or which may be optionally monosubstituted by a substituent selected from the following (a) to (e):

(a) a —C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;

(b) a —C₁₋₃ alkylene-O—C₁₋₆ alkyl group which may be optionally substituted by 1 to 5 identical or different substituents selected from deuterium atoms, halogen atoms, C₁₋₃ alkoxy groups, and C₁₋₃ haloalkoxy groups;

(c) a —C₁₋₃ alkylene-O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;

(d) an —O—C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups; and

(e) an —O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups.

<5> The compound according to any one of the above-described <1> to <4> or a pharmaceutically acceptable salt thereof, wherein

R₃ is a C₃₋₆ alkyl group or a cycloalkyl group having 3 to 7 carbon atoms.

<6> The compound according to any one of the above-described <1> to <5> or a pharmaceutically acceptable salt thereof, wherein

Y is —S—, —O—, or —NH—.

<7> The compound according to any one of the above-described <1> to <6> or a pharmaceutically acceptable salt thereof, wherein

Z is a single bond or a C₁₋₃ alkylene group.

<8> The compound according to any one of the above-described <1> to <7> or a pharmaceutically acceptable salt thereof, wherein

R₄ is a C₁₋₆ alkyl group, a phenyl group, a cycloalkyl group having 3 to 7 carbon atoms, or a 5- or 6-membered saturated or unsaturated heterocyclic group.

<9> The compound according to the above-described <1> or a pharmaceutically acceptable salt thereof, wherein

the compound is selected from the group consisting of the following compounds:

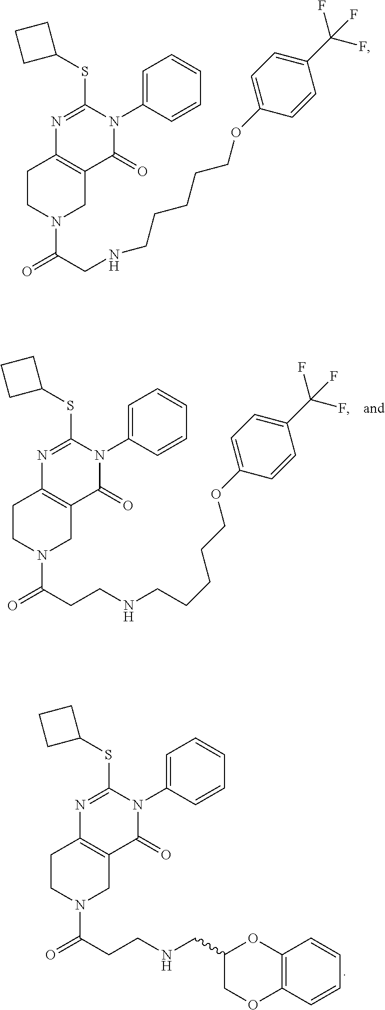 [SMILES: O=C(CNCCCCCOc1ccc(C(F)(F)F)cc1)N1CCc2nc(SC3CCC3)n(-c3ccccc3)c(=O)c2C1 ; O=C(CCNCCCCCOc1ccc(C(F)(F)F)cc1)N1CCc2nc(SC3CCC3)n(-c3ccccc3)c(=O)c2C1 ; O=C(CCNCC1COc2ccccc2O1)N1CCc2nc(SC3CCC3)n(-c3ccccc3)c(=O)c2C1]

<10> A monoacylglycerol acyltransferase (MGAT) inhibitor, comprising the compound according to any one of the above-described <1> to <9> or a pharmaceutically acceptable salt thereof as an active ingredient.

<11> A fat absorption inhibitor, comprising the compound according to any one of the above-described <1> to <9> or a pharmaceutically acceptable salt thereof as an active ingredient.

<12> An agent for preventing and/or treating adiposity, comprising the compound according to any one of the above-described <1> to <9> or a pharmaceutically acceptable salt thereof as an active ingredient.

<13> An agent for preventing and/or treating a lipid metabolism disorder, comprising the compound according to any one of the above-described <1> to <9> or a pharmaceutically acceptable salt thereof as an active ingredient.

<14> A pharmaceutical composition, comprising the compound according to any one of the above-described <1> to <9> or a pharmaceutically acceptable salt thereof as an active ingredient.

<15> A method for producing a compound represented by the following general formula (I) or a pharmaceutically acceptable salt thereof, comprising the steps of:

(a) adding acryloyl chloride to a compound represented by the following general formula (II) for a reaction therebetween, to obtain a compound represented by the following general formula (III); and

(b) adding an alkylated amine represented by Formula L-X—R₂—NH₂, where L, X, and R₂ are the same as L, X, and R₂ in general formula (I), respectively, to the compound represented by general formula (III) for a reaction therebetween, to obtain the compound represented by general formula (I):

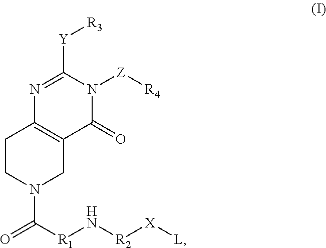

wherein

R₁ represents a linear ethylene group which may be optionally substituted by deuterium atoms;

R₂ represents a linear C₁₋₆ alkylene group or acyclic C₃₋₆ alkylene group, which may be optionally substituted by 1 to 6 identical or different substituents selected from deuterium atoms, halogen atoms, C₁₋₃ alkyl groups, C₁₋₃ haloalkyl groups, C₁₋₃ alkylthio groups, nitrile groups, hydroxyl groups, C₁₋₃ alkoxy groups, C₁₋₃ haloalkoxy groups, amino groups, C₁₋₃ alkylamino groups, and C₂₋₆ dialkylamino groups;

X represents a single bond, —O—, —S—, or —NR₅—, where R₅ represents a hydrogen atom, a C₁₋₆ alkyl group, or a C₁₋₆ haloalkyl group;

L represents a C₂₋₆ alkenyl group, a C₂₋₆ alkynyl group, a cycloalkyl group having 3 to 6 carbon atoms, a phenyl group, a naphthyl group, a 3- to 6-membered saturated or unsaturated heterocyclic group, or a 9- or 10-membered saturated or unsaturated heterobicyclic group, which may be optionally substituted by 1 to 5 identical or different substituents selected from deuterium atoms, halogen atoms, hydroxyl groups, amino groups, C₁₋₃ alkyl groups, C₁₋₃ haloalkyl groups, C₁₋₃ alkylthio groups, nitrile groups, C₁₋₃ alkoxy groups, C₁₋₃ haloalkoxy groups, C₁₋₃ alkylamino groups, and C₂₋₆ dialkylamino groups, or which may be optionally monosubstituted by a substituent selected from the following (a) to (e):

(a) a —C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;

(b) a —C₁₋₃ alkylene-O—C₁₋₆ alkyl group which may be optionally substituted by 1 to 5 identical or different substituents selected from deuterium atoms, halogen atoms, C₁₋₃ alkoxy groups, and C₁₋₃ haloalkoxy groups;

(c) a —C₁₋₃ alkylene-O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;

(d) an —O—C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups; and

(e) an —O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;

Y represents a single bond, —S—, —O—, or —NR₆—, where R₆ represents a hydrogen atom, a C₁₋₆ alkyl group, or a C₁₋₆ haloalkyl group;

Z represents a single bond or a C₁₋₆ alkylene group;

R₃ represents a C₃₋₈ alkyl group, a C₃₋₈ cycloalkyl group, or a 3- to 8-membered saturated or unsaturated heterocyclic group, and may optionally have 1 to 7 identical or different substituents selected from the group consisting of deuterium atoms, halogen atoms, C₁₋₃ alkyl groups, C₁₋₃ haloalkoxy groups, and C₁₋₃ haloalkyl groups; and

R₄ represents a C₁₋₆ alkyl group, a phenyl group, a cycloalkyl group having 3 to 8 carbon atoms, or a 3- to 8-membered saturated or unsaturated heterocyclic group, and may optionally have 1 to 7 identical or different substituents selected from the group consisting of deuterium atoms, halogen atoms, C₁₋₃ alkyl groups, C₁₋₃ haloalkyl groups, C₁₋₃ alkoxy groups, and C₁₋₃ haloalkoxy groups;

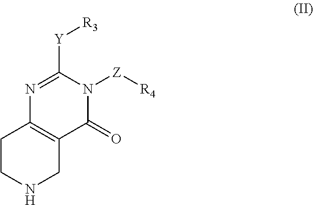

wherein

Y, Z, R₃, and R₄ are the same as Y, Z, R₃, and R₄ in general formula (I), respectively; and

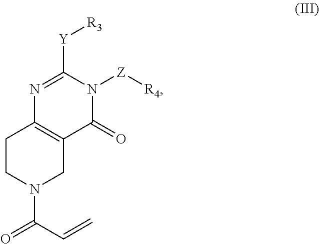

wherein

Y, Z, R₃, and R₄ are the same as Y, Z, R₃, and R₄ in general formula (I), respectively.

### Advantageous Effects of Invention

The compound represented by general formula (I) of the present invention or a pharmaceutically acceptable salt thereof has an extremely higher MGAT inhibitory activity than conventional compounds. Accordingly, the compound represented by general formula (I) of the present invention or a pharmaceutically acceptable salt thereof is useful as an MGAT inhibitor, and can be used suitably for inhibiting fat absorption or treating or preventing adiposity.

### DESCRIPTION OF EMBODIMENTS

### Definitions

In Description and Claims, a “C₁₋₆ alkyl group” and a “C₁₋₆ alkylene group” mean an alkyl group having 1 to 6 carbon atoms and an alkylene group having 1 to 6 carbon atoms, respectively.

In Description and Claims, a “haloalkyl group” means a group (halogenated alkyl group) in which some or all hydrogen atoms constituting an alkyl group are replaced with halogen atoms. Likewise, a “haloalkoxy group” means a group (halogenated alkoxy group) in which some or all hydrogen atoms constituting an alkoxy group are replaced with halogen atoms.

Meanwhile, in Description and Claims, “halogen atoms” are a concept including fluorine atoms, chlorine atoms, bromine atoms, and iodine atoms.

In Description and Claims, the “alkyl group”, the “alkylene group,” and the “alkoxy group” (including the alkyl group and the alkoxy group constituting the haloalkyl group, the haloalkoxy group, or the like) may be linear or branched, unless otherwise noted.

In Description and Claims, a “heterocyclic group” means a group obtained by removing one hydrogen atom from a saturated or unsaturated ring (heterocycle) containing 1 to 3 heteroatoms selected from oxygen atoms, sulfur atoms, and nitrogen atoms.

Meanwhile, in Description and Claims, a “heterobicyclic group” means a group obtained by removing one hydrogen atom from a condensed ring obtained by condensing two rings including a heterocycle.

Examples of saturated heterocyclic groups include groups obtained by removing one hydrogen atom from piperidine, piperazine, pyrrolidine, tetrahydrofuran, and the like.

Examples of unsaturated heterocyclic groups include groups obtained by removing one hydrogen atom from thiophene, furan, oxazole, thiazole, oxadiazole, pyridine, and the like.

Examples of unsaturated heterobicyclic groups include groups obtained by removing one hydrogen atom from indole, indoline, benzothiophene, benzofuran, benzoxazole, benzodioxazole, and the like.

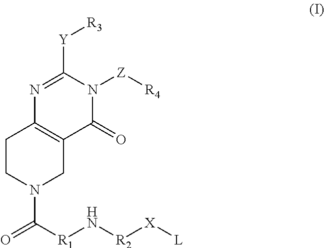

Hereinafter, a compound represented by general formula (I) of the present invention and a pharmaceutically acceptable salt thereof is described.

In general formula (I), R₁ represents a linear C₁₋₆ alkylene group or a cyclic C₃₋₆ alkylene group, which may be optionally substituted by deuterium atoms. R₁ is more preferably a linear C₁₋₃ alkylene group, and further preferably a linear C₁₋₂ alkylene group. Specifically, a methylene group or an ethylene group is particularly preferably used as R₁.

In general formula (I), R₂ represents a linear C₁₋₆ alkylene group, which may be optionally substituted by 1 to 6 identical or different substituents selected from deuterium atoms, halogen atoms, C₁₋₃ alkyl groups, C₁₋₃ haloalkyl groups, C₁₋₃ alkylthio groups, nitrile groups, hydroxyl groups, C₁₋₃ alkoxy groups, C₁₋₃ haloalkoxy groups, amino groups, C₁₋₃ alkylamino groups, and C₂₋₆ dialkylamino groups.

R₂ is preferably a linear C₁₋₆ alkylene group, and more preferably a linear C₁₋₅ alkylene group. Specifically, a methylene group, an ethylene group, a n-propylene group, a n-butylene group, or a n-pentylene group is particularly preferable.

When R₂ has a substituent(s), the substituent is preferably a C₁₋₃ alkyl group. A hydroxyl group is also preferable as the substituent. The number of the substituents is preferably 1 to 3, and more preferably 1 or 2.

In general formula (I), X represents a single bond, —O—, —S—, or —NR₅—, where R₅ represents a hydrogen atom, a C₁₋₆ alkyl group, or a C₁₋₆ haloalkyl group.

X is preferably a single bond, —O—, —S—, or —NH—, more preferably a single bond, —O—, or —NH—, and further preferably a single bond or —O—.

In general formula (I), L represents a C₂₋₆ alkenyl group, a C₂₋₆ alkynyl group, a cycloalkyl group having 3 to 6 carbon atoms, a phenyl group, a naphthyl group, a 3- to 6-membered saturated or unsaturated heterocyclic group, or a 9- or 10-membered saturated or unsaturated heterobicyclic group. If necessary, these groups serving as L may be substituted by 1 to 5 identical or different substituents selected from deuterium atoms, halogen atoms, hydroxyl groups, amino groups, C₁₋₃ alkyl groups, C₁₋₃ haloalkyl groups, C₁₋₃ alkylthio groups, nitrile groups, C₁₋₃ alkoxy groups, C₁₋₃ haloalkoxy groups, C₁₋₃ alkylamino groups, and C₂₋₆ dialkylamino groups, or may be monosubstituted by a substituent selected from the following (a) to (e):

(a) a —C₁₋₃ alkylene-phenyl group which may be optionally substituted by 1 to 5, preferably 1 to 3, and more preferably or 2 identical or different substituents, or further preferably one substituent, selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;

(b) a —C₁₋₃ alkylene-O—C₁₋₆ alkyl group which may be optionally substituted by 1 to 5, preferably 1 to 3, and more preferably 1 or 2 identical or different substituents, or further preferably one substituent, selected from deuterium atoms, halogen atoms, C₁₋₃ alkoxy groups, and C₁₋₃ haloalkoxy groups;

(c) a —C₁₋₃ alkylene-O-phenyl group which may be optionally substituted by 1 to 5, preferably 1 to 3, and more preferably 1 or 2 identical or different substituents, or further preferably one substituent, selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;

(d) an —O—C₁₋₃ alkylene-phenyl group which may be optionally substituted by 1 to 5, preferably 1 to 3, and more preferably 1 or 2 identical or different substituents, or further preferably one substituent, selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups; and

(e) an —O-phenyl group which may be optionally substituted by 1 to 5, preferably 1 to 3, and more preferably or 2 identical or different substituents, or further preferably one substituent, selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups.

Of these groups, L is preferably a phenyl group, a naphthyl group, a 5- or 6-membered saturated or unsaturated heterocyclic group, or a 9- or 10-membered saturated or unsaturated heterobicyclic group, and more preferably a group having any of the following structures.

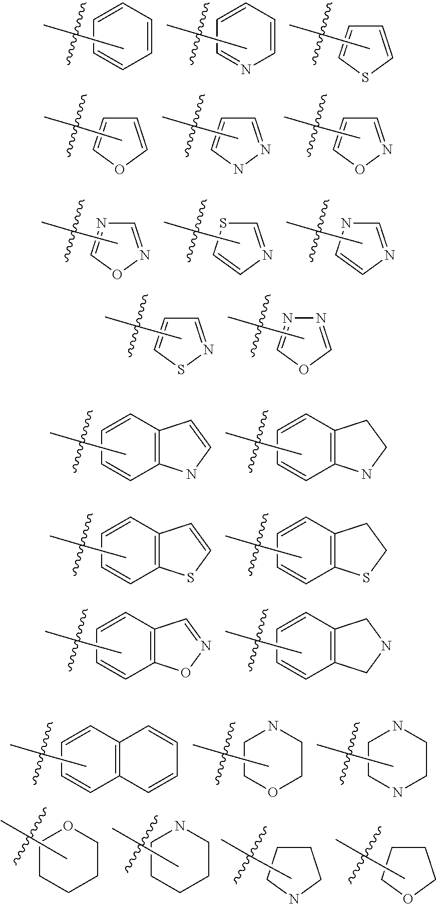

The position at which the above-described group is bonded to X is not particularly limited, as long as the group having the structure can be bonded to X at that position. In addition, in the case of a heterobicycle, any one of the two condensed rings may be bonded to X.

Moreover, a C₂₋₆ alkynyl group (preferably an ethynyl group) or a group having any of the following structure is also preferable as L.

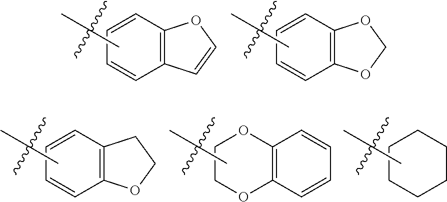

The position at which the above-described group is bonded to X is not particularly limited, as long as the group having the structure can be bonded to X at that position. In addition, in the case of a heterobicycle, any one of the two condensed rings may be bonded to X.

Of these groups, L is particularly preferably a phenyl group, or a group obtained by removing one hydrogen atom from thiophene, furan, indole, indoline, thiophene, thiazole, pyrazole, oxazole, or oxadiazole.

Moreover, the following group is also particularly preferable as L.

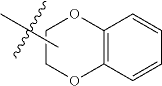

When L has a substituent(s), the number of the substituents is 1 to 5, preferably 1 to 3, and more preferably 1 or 2.

In addition, when L has a substituent(s), L is preferably substituted by 1 or 2 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups, or preferably monosubstituted by a substituent selected from the following (a) to (e):

(a) a —C₁₋₃ alkylene-phenyl group which may be optionally substituted by 1 or 2 identical or different substituents, or more preferably 1 substituent, selected from deuterium atoms, halogen atoms, C₁₋₃ alkyl groups, C₁₋₃ haloalkyl groups, C₁₋₃ alkylthio groups, and nitrile groups (preferably selected from C₁₋₃ haloalkyl groups, and more preferably CF₃);

(b) a —C₁₋₃ alkylene-O—C₁₋₆ alkyl group which may be optionally substituted by 1 or 2 identical or different substituents, or more preferably 1 substituent, selected from deuterium atoms and halogen atoms;

(c) a —C₁₋₃ alkylene-O-phenyl group which may be optionally substituted by 1 or 2 identical or different substituents, or more preferably 1 substituent, selected from deuterium atoms, halogen atoms, C₁₋₃ alkyl groups, C₁₋₃ haloalkyl groups, C₁₋₃ alkylthio groups, and nitrile groups (preferably selected from C₁₋₃ haloalkyl groups, and more preferably CF₃);

(d) an —O—C₁₋₃ alkylene-phenyl group which may be optionally substituted by 1 or 2 identical or different substituents, or more preferably 1 substituent, selected from deuterium atoms, halogen atoms, C₁₋₃ alkyl groups, C₁₋₃ haloalkyl groups, C₁₋₃ alkylthio groups, and nitrile groups (preferably selected from C₁₋₃ haloalkyl groups, and more preferably CF₃); and

(e) an —O-phenyl group which may be optionally substituted by 1 or 2 identical or different substituents, or more preferably 1 substituent, selected from deuterium atoms, halogen atoms, C₁₋₃ alkyl atoms, C₁₋₃ haloalkyl groups, C₁₋₃ alkylthio groups, and nitrile groups (preferably selected from C₁₋₃ haloalkyl groups, and more preferably CF₃).

Of these groups, each substituent of L is preferably a C₁₋₃ haloalkyl group, a C₁₋₃ haloalkoxy group, or a halogen atom, more preferably —CF₃, —O—CF₃, or a halogen atom, and further preferably —CF₃.

In addition, L is preferably a phenyl group which may be optionally substituted by 1 to 5 and preferably 1 or 2 identical or different substituents, or more preferably 1 substituent, selected from CF₃ and halogen atoms; an ethynyl group which may be optionally substituted by an —O-phenyl group which may be optionally substituted by 1 or 2 identical or different C₁₋₃ haloalkyl groups, preferably one C₁₋₃ haloalkyl group, and preferably CF₃; or any one of the following groups. L is more preferably a phenyl group in which the hydrogen at the para position may be replaced by CF₃ or a halogen atom, and preferably by CF₃.

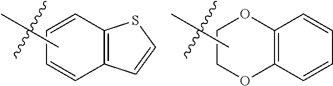

In general formula (I), Y represents a single bond, —S—, —O—, or —NR⁶—, where R₆ represents a hydrogen atom, a C₁₋₆ alkyl group, or a C₁₋₆ haloalkyl group.

Of these groups, Y is preferably —S—, —O—, or —NH—, more preferably —S— or —NH—, and further preferably —S—.

In general formula (I), Z represents a single bond or a C₁₋₆ alkylene group.

Z is preferably a single bond or a C₁₋₃ alkylene group, more preferably a single bond or a C₁₋₂ alkylene group, and further preferably a single bond or a methylene group.

In general formula (I), R₃ represents a C₃₋₈ alkyl group, a cycloalkyl group having 3 to 8 carbon atoms, or a 3- to 8-membered saturated or unsaturated heterocyclic group. If necessary, these groups serving as R₃ may have 1 to 7 identical or different substituents selected from the group consisting of deuterium atoms, halogen atoms, C₁₋₃ alkyl groups, C₁₋₃ haloalkoxy groups, and C₁₋₃ haloalkyl groups.

R₃ is preferably a C₃₋₆ alkyl group or a cycloalkyl group having 3 to 7 carbon atoms, more preferably a cycloalkyl group having 3 to 6 carbon atoms, further preferably a cycloalkyl group having 4 to 5 carbon atoms, and particularly preferably a cycloalkyl group having 4 carbon atoms.

When R₃ has a substituent(s), the number of the substituents is 1 to 7, preferably 1 to 6, more preferably 1 to 4, further preferably 1 or 2, and particularly preferably 1. An unsubstituted group is also as preferable as R₃.

In general formula (I), R₄ represents a C₁₋₆ alkyl group, a phenyl group, a cycloalkyl group having 3 to 8 carbon atoms, or a 3- to 8-membered saturated or unsaturated heterocyclic group. If necessary, each of these groups may have 1 to 7 identical or different substituents selected from the group consisting of deuterium atoms, halogen atoms, C₁₋₃ alkyl groups, C₁₋₃ haloalkyl groups, C₁₋₃ alkoxy groups, and C₁₋₃ haloalkoxy groups.

R₄ is preferably a C₁₋₆ alkyl group, a phenyl group, a cycloalkyl group having 3 to 7 carbon atoms, or a 5- or 6-membered saturated or unsaturated heterocyclic group, and more preferably a group selected from the following groups:

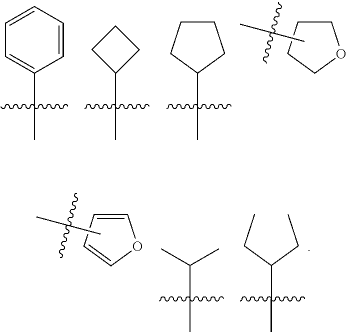

Of the above-described groups, the furanyl group or the tetrahydrofuranyl group may be bonded to Z at any position without any particular limitation, as long as the group having the structure can be bonded to Z at that position.

Preferred specific examples of the compound represented by general formula (I) are listed below. However, the compound of the present invention is not limited to these compounds.
2-cyclobutylsulfanyl-3-phenyl-6-[2-[[4-(trifluoromethyl)phenyl]methylamino]acetyl]-7,8-dihydro-5H-pyrido[4,3-d]pyrimidin-4-one
2-cyclobutylsulfanyl-3-phenyl-6-[2-(3-phenylpropylamino)acetyl]-7,8-dihydro-5H-pyrido[4,3-d]pyrimidin-4-one
6-[2-[(4-chlorophenyl)methylamino]acetyl]-2-cyclobutylsulfanyl-3-phenyl-7,8-dihydro-5H-pyrido[4,3-d]pyrimidin-4-one
6-[2-[(3-chlorophenyl)methylamino]acetyl]-2-cyclobutylsulfanyl-3-phenyl-7,8-dihydro-5H-pyrido[4,3-d]pyrimidin-4-one
2-cyclobutylsulfanyl-3-phenyl-6-[2-[5-[4-(trifluoromethyl)phenoxy]pentylamino]acetyl]-7,8-dihydro-5H-pyrido[4,3-d]pyrimidin-4-one
2-cyclobutylsulfanyl-3-phenyl-6-[2-[[4-[[4-(trifluoro methyl)phenyl]methoxy]phenyl]methylamino]acetyl]-7,8-dihydro-5H-pyrido[4,3-d]pyrimidin-4-one
2-cyclobutylsulfanyl-6-[2-[[4-(phenoxymethyl)phenyl]methylamino]acetyl]-3-phenyl-7,8-dihydro-5H-pyrido[4,3-d]pyrimidin-4-one
2-cyclobutylsulfanyl-6-[2-(phenethylamino)acetyl]-3-phenyl-7,8-dihydro-5H-pyrido[4,3-d]pyrimidin-4-one
2-cyclobutylsulfanyl-6-[2-[2-(3,4-difluorophenoxyl)ethylamino]acetyl]-3-phenyl-7,8-dihydro-5H-pyrido[4,3-d]pyrimidin-4-one
6-[3-(benzylamino)propanoyl]-2-cyclobutylsulfanyl-3-phenyl-7,8-dihydro-5H-pyrido[4,3-d]pyrimidin-4-one
6-[3-(benzylamino)propanoyl]-2-cyclobutylsulfanyl-3-(2,3,4,5,6-pentadeuteriophenyl)-7,8-dihydro-5H-pyrido[4,3-d]pyrimidin-4-one
6-[3-(benzylamino)propanoyl]-3-(cyclobutylmethyl)-2-cyclobutylsulfanyl-7,8-dihydro-5H-pyrido[4,3-d]pyrimidin-4-one
6-[3-(benzylamino)propanoyl]-2-cyclobutylsulfanyl-3-[[(2R)-tetrahydrofuran-2-yl]methyl]-7,8-dihydro-5H-pyrido[4,3-d]pyrimidin-4-one
6-[3-(benzylamino)propanoyl]-2-cyclobutylsulfanyl-3-(2-furylmethyl)-7,8-dihydro-5H-pyrido[4,3-d]pyrimidin-4-one
6-[3-(benzylamino)propanoyl]-2-cyclobutylsulfanyl-3-cyclopentyl-7,8-dihydro-5H-pyrido[4,3-d]pyrimidin-4-one
2-cyclobutylsulfanyl-3-phenyl-6-[3-(3-thienylmethylamino)propanoyl]-7,8-dihydro-5H-pyrido[4,3-d]pyrimidin-4-one
2-cyclobutylsulfanyl-6-[3-[(2-methoxyphenyl)methylamino]propanoyl]-3-phenyl-7,8-dihydro-5H-pyrido[4,3-d]pyrimidin-4-one
2-cyclobutylsulfanyl-6-[3-[(2-fluorophenyl)methylamino]propanoyl]-3-phenyl-7,8-dihydro-5H-pyrido[4,3-d]pyrimidin-4-one
2-cyclobutylsulfanyl-3-phenyl-6-[3-[(4-trifluoromethyl)phenyl)methylamino]propanoyl]-7,8-dihydro-5H-pyrido[4,3-d]pyrimidin-4-one
6-[3-[(benzothiophen-3-ylmethylamino)propanoyl]-2-cyclobutylsulfanyl-3-phenyl-7,8-dihydro-5H-pyrido[4,3-d]pyrimidin-4-one
2-cyclobutylsulfanyl-6-[3-[(1H-indol-2-ylmethylamino) propanoyl]-3-phenyl-7,8-dihydro-5H-pyrido[4,3-d]pyrimidin-4-one
2-cyclobutylsulfanyl-3-phenyl-6-[4-[[4-(trifluoromethyl)phenyl]methylamino]butanoyl]-7,8-dihydro-5H-pyrido[4,3-d]pyrimidin-4-one
2-cyclobutylsulfanyl-6-[3-[2-(1H-indol-3-yl)ethylamin o]propanoyl]-3-phenyl-7,8-dihydro-5H-pyrido[4,3-d]pyrimidin-4-one
2-cyclobutylsulfanyl-3-phenyl-6-[3-[[(4-trifluoromethoxy)phenyl]methylamino]propanoyl]-7,8-dihydro-5H-pyrido[4,3-d]pyrimidin-4-one
2-cyclobutylsulfanyl-6-[3-[[dideuterio-(2,3,4,5,6-pentadeuteriophenyl)methyl]amino]propanoyl]-3-phenyl-7,8-dihydro-5H-pyrido[4,3-d]pyrimidin-4-one
2-cyclobutylsulfanyl-6-[3-[(2-methylbenzofuran-7-yl)methylamino]propanoyl]-3-phenyl-7,8-dihydro-5H-pyrido[4,3-d]pyrimidin-4-one
6-[3-[(3-chloro-4-fluoro-phenyl)methylamino]propanoyl]-2-cyclobutylsulfanyl-3-phenyl-7,8-dihydro-5H-pyrido[4,3-d]pyrimidin-4-one
6-[3-(1,3-benzodioxol-5-ylmethylamino)propanoyl]-2-cyclobutylsulfanyl-3-phenyl-7,8-dihydro-5H-pyrido[4,3-d]pyrimidin-4-one
2-cyclobutylsulfanyl-6-[3-[(2-hydroxy-2-phenyl-ethyl)amino]propanoyl]-3-phenyl-7,8-dihydro-5H-pyrido[4,3-d]pyrimidin-4-one
2-cyclobutylsulfanyl-6-[3-[2-(4-hydroxyphenyl)ethylamino]propanoyl]-3-phenyl-7,8-dihydro-5H-pyrido[4,3-d]pyrimidin-4-one
2-cyclobutylsulfanyl-6-[3-[(4-dimethylaminophenyl)methylamino]propanoyl]-3-phenyl-7,8-dihydro-5H-pyrido[4,3-d]pyrimidin-4-one
2-cyclobutylsulfanyl-6-[3-(2,3-dihydrobenzofuran-5-ylmethylamino)propanoyl]-3-phenyl-7,8-dihydro-5H-pyrido[4,3-d]pyrimidin-4-one
2-cyclobutylsulfanyl-3-phenyl-6-[3-[5-[4-(trifluoromethyl)phenoxy]pentylamino]propanoyl]-7,8-dihydro-5H-pyrido[4,3-d]pyrimidin-4-one
6-[3-[(3-benzyl-1,2,4-oxadiazol-5-yl)methylamino]propanoyl]-2-cyclobutylsulfanyl-3-phenyl-7,8-dihydro-5H-pyrido[4,3-d]pyrimidin-4-one
2-cyclobutylsulfanyl-6-[3-[(2,6-difluorophenyl)methyl amino]propanoyl]-3-phenyl-7,8-dihydro-5H-pyrido[4,3-d]pyrimidin-4-one
2-cyclobutylsulfanyl-3-phenyl-6-[3-[[(2R)-2-phenylpropyl]amino]propanoyl]-7,8-dihydro-5H-pyrido[4,3-d]pyrimidin-4-one
2-cyclobutylsulfanyl-3-phenyl-6-[3-[[(2S)-2-phenylpropyl]amino]propanoyl]-7,8-dihydro-5H-pyrido[4,3-d]pyrimidin-4-one
6-[3-(2-anilinoethylamino)propanoyl]-2-cyclobutylsulfanyl-3-phenyl-7,8-dihydro-5H-pyrido[4,3-d]pyrimidin-4-one
2-cyclobutylsulfanyl-3-phenyl-6-[3-[[3-(trifluoromethyl)cyclohexyl]methylamino]propanoyl]-7,8-dihydro-5H-pyrido[4,3-d]pyrimidin-4-one
6-[3-[[(2R)-2-(4-chloroanilino)-3-methyl-butyl]amino]propanoyl]-2-cyclobutylsulfanyl-3-phenyl-7,8-dihydro-5H-pyrido[4,3-d]pyrimidin-4-one
2-cyclobutylsulfanyl-3-phenyl-6-[3-[[(1R)-1-phenyleth yl]amino]propanoyl]-7,8-dihydro-5H-pyrido[4,3-d]pyrimidin-4-one
2-cyclobutylsulfanyl-3-phenyl-6-[3-[[(1S)-1-phenyleth yl]amino]propanoyl]-7,8-dihydro-5H-pyrido[4,3-d]pyrimidin-4-one
2-cyclobutylsulfanyl-6-[3-[[(2R)-2-(4-fluorophenyl)-2-hydroxy-ethyl]amino]propanoyl]-3-phenyl-7,8-dihydro-5H-pyrido[4,3-d]pyrimidin-4-one
2-cyclobutylsulfanyl-6-[3-[[3-(4-fluorophenoxy)-2-hydroxy-propyl]amino]propanoyl]-3-phenyl-7,8-dihydro-5H-pyrido[4,3-d]pyrimidin-4-one
6-[3-[[3-(4-chlorophenoxy)-2-hydroxy-propyl]amino]propanoyl]-2-cyclobutylsulfanyl-3-phenyl-7,8-dihydro-5H-pyrido[4,3-d]pyrimidin-4-one
2-cyclobutylsulfanyl-6-[3-[2-(4-fluorophenoxy)ethylamino]propanoyl]-3-phenyl-7,8-dihydro-5H-pyrido[4,3-d]pyrimidin-4-one
2-cyclobutylsulfanyl-6-[3-[3-(4-fluorophenoxy)propylamino]propanoyl]-3-phenyl-7,8-dihydro-5H-pyrido[4,3-d]pyrimidin-4-one
2-cyclobutylsulfanyl-6-[3-[4-(4-fluorophenoxy)butylamino]propanoyl]-3-phenyl-7,8-dihydro-5H-pyrido[4,3-d]pyrimidin-4-one
2-cyclobutylsulfanyl-6-[3-[5-(4-fluorophenoxyl)pentylamino]propanoyl]-3-phenyl-7,8-dihydro-5H-pyrido[4,3-d]pyrimidin-4-one
2-cyclobutylsulfanyl-6-[2-[3-(4-fluorophenoxy)propylamino]acetyl]-3-phenyl-7,8-dihydro-5H-pyrido[4,3-d]pyrimidin-4-one
2-cyclobutylsulfanyl-6-[2-[2-(4-fluorophenoxy)ethylamino]acetyl]-3-phenyl-7,8-dihydro-5H-pyrido[4,3-d]pyrimidin-4-one
2-cyclobutylsulfanyl-6-[2-[4-(4-fluorophenoxy)butylamino]acetyl]-3-phenyl-7,8-dihydro-5H-pyrido[4,3-d]pyrimidin-4-one
2-cyclobutylsulfanyl-6-[2-[5-(4-fluorophenoxy)pentylamino]acetyl]-3-phenyl-7,8-dihydro-5H-pyrido[4,3-d]pyrimidin-4-one
2-cyclobutylsulfanyl-3-phenyl-6-[2-[[4-[3-(trifluoromethyl)phenoxy]phenyl]methylamino]acetyl]-7,8-dihydro-5H-pyrido[4,3-d]pyrimidin-4-one
2-cyclobutylsulfanyl-3-phenyl-6-[2-[5-[4-(trifluoromethyl)anilino]pentylamino]acetyl]-7,8-dihydro-5H-pyrido[4,3-d]pyrimidin-4-one
2-cyclobutylsulfanyl-3-phenyl-6-[3-[[5-[[4-(trifluoro methyl)phenyl]methyl]-1,3,4-oxadiazol-2-yl]methylamino]propanoyl]-7,8-dihydro-5H-pyrido[4,3-d]pyrimidin-4-one
2-cyclobutylsulfanyl-6-[2-[5-(cyclohexoxy)pentylamino]acetyl]-3-phenyl-7,8-dihydro-5H-pyrido[4,3-d]pyrimidin-4-one
2-cyclobutylsulfanyl-6-[2-[5-(4,4-difluorocyclohexoxyl)pentylamino]acetyl]-3-phenyl-7,8-dihydro-5H-pyrido[4,3-d]pyrimidin-4-one
2-cyclobutylsulfanyl-3-phenyl-6-[3-[4-[4-(trifluoromethyl)phenoxy]but-2-ynylamino]propanoyl]-7,8-dihydro-5H-pyrido[4,3-d]pyrimidin-4-one
2-cyclobutylsulfanyl-6-[3-(2,3-dihydro-1,4-benzodioxin-3-ylmethylamino)propanoyl]-3-phenyl-7,8-dihydro-5H-pyrido[4,3-d]pyrimidin-4-one

Of these compounds, the compound represented by general formula (I) is particularly preferably a compound selected from the group consisting of the following compounds:
2-cyclobutylsulfanyl-3-phenyl-6-[2-[5-[4-(trifluoromethyl)phenoxy]pentylamino]acetyl]-7,8-dihydro-5H-pyrido[4,3-d]pyrimidin-4-one

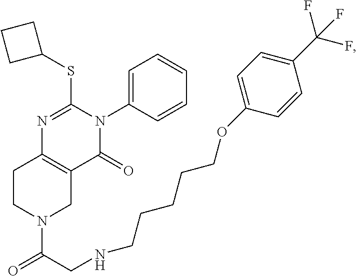 [SMILES: O=C(CNCCCCCOc1ccc(C(F)(F)F)cc1)N1CCc2nc(SC3CCC3)n(-c3ccccc3)c(=O)c2C1]

2-cyclobutylsulfanyl-3-phenyl-6-[3-[5-[4-(trifluoromethyl)phenoxy]pentylamino]propanoyl]-7,8-dihydro-5H-pyrido[4,3-d]pyrimidin-4-one

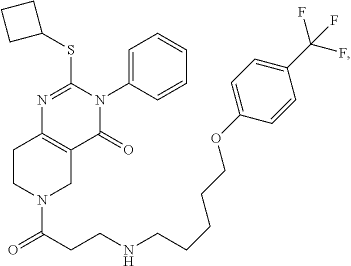 [SMILES: O=C(CCNCCCCCOc1ccc(C(F)(F)F)cc1)N1CCc2nc(SC3CCC3)n(-c3ccccc3)c(=O)c2C1]

and
2-cyclobutylsulfanyl-6-[3-(2,3-dihydro-1,4-benzodioxin-3-ylmethylamino)propanoyl]-3-phenyl-7,8-dihydro-5H-pyrido
[4,3-d]pyrimidin-4-one

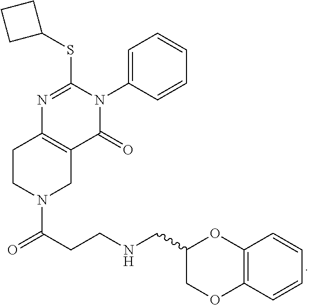 [SMILES: O=C(CCNCC1COc2ccccc2O1)N1CCc2nc(SC3CCC3)n(-c3ccccc3)c(=O)c2C1]

Of these compounds, 2-cyclobutylsulfanyl-3-phenyl-6-[2-[5-[4-(trifluoromethyl)phenoxy]pentylamino]acetyl]-7,8-dihydro-5H-pyrido[4,3-d]pyrimidin-4-one or 2-cyclobutylsulfanyl-3-phenyl-6-[3-[5-[4-(trifluoromethyl)phenoxy]pentylamino]propanoyl]-7,8-dihydro-5H-pyrido[4,3-d]pyrimidin-4-one is particularly preferable, and 2-cyclobutylsulfanyl-3-phenyl-6-[2-[5-[4-(trifluoromethyl)phenoxy]pentylamino]acetyl]-7,8-dihydro-5H-pyrido[4,3-d]pyrimidin-4-one is the most preferable.

In addition, from the viewpoint of the MGAT2 inhibitory action, the following compounds are also preferably used:
2-cyclobutylsulfanyl-3-phenyl-6-[2-[5-[4-(trifluoromethyl)phenoxy]pentylamino]acetyl]-7,8-dihydro-5H-pyrido[4,3-d]pyrimidin-4-one,
6-[3-[(benzylamino)propanoyl]-2-cyclobutylsulfanyl-3-phenyl-7,8-dihydro-5H-pyrido[4,3-d]pyrimidin-4-one,
2-cyclobutylsulfanyl-3-phenyl-6-[3-[(4-trifluoromethyl)phenyl]methylamino]propanoyl]-7,8-dihydro-5H-pyrido[4,3-d]pyrimidin-4-one,
6-[3-[(benzothiophen-3-ylmethylamino)propanoyl]-2-cyclobutylsulfanyl-3-phenyl-7,8-dihydro-5H-pyrido[4,3-d]pyrimidin-4-one,
6-[3-(2-anilinoethylamino)propanoyl]-2-cyclobutylsulfanyl-3-phenyl-7,8-dihydro-5H-pyrido[4,3-d]pyrimidin-4-one,
2-cyclobutylsulfanyl-6-[3-[[3-(4-fluorophenoxy)-2-hydroxy-propyl]amino]propanoyl]-3-phenyl-7,8-dihydro-5H-pyrido[4,3-d]pyrimidin-4-one,
6-[3-[[3-(4-chlorophenoxy)-2-hydroxy-propyl]amino]propanoyl]-2-cyclobutylsulfanyl-3-phenyl-7,8-dihydro-5H-pyrido[4,3-d]pyrimidin-4-one,
2-cyclobutylsulfanyl-6-[2-[3-(4-fluorophenoxy)propylamino]acetyl]-3-phenyl-7,8-dihydro-5H-pyrido[4,3-d]pyrimidin-4-one,
2-cyclobutylsulfanyl-6-[2-[2-(4-fluorophenoxy)ethylamino]acetyl]-3-phenyl-7,8-dihydro-5H-pyrido[4,3-d]pyrimidin-4-one,
2-cyclobutylsulfanyl-6-[2-[4-(4-fluorophenoxy)butylamino]acetyl]-3-phenyl-7,8-dihydro-5H-pyrido[4,3-d]pyrimidin-4-one,
2-cyclobutylsulfanyl-6-[2-[5-(4-fluorophenoxy)pentylamino]acetyl]-3-phenyl-7,8-dihydro-5H-pyrido[4,3-d]pyrimidin-4-one, and
2-cyclobutylsulfanyl-3-phenyl-6-[3-[4-[4-(trifluoromethyl)phenoxy]but-2-ynylamino]propanoyl]-7,8-dihydro-5H-pyrido[4,3-d]pyrimidin-4-one.

Examples of pharmaceutically acceptable salts of the compound represented by general formula (I) include acid addition salts, alkali metal salts, and alkaline earth metal salts.

The acid addition salts may be organic acid salts or inorganic acid salts. The organic acid salts include trifluoroacetate, oxalate, maleate, fumarate, malonate, lactate, malate, citrate, tartrate, methanesulfonate, p-toluenesulfonate, and the like. The inorganic acid salts include hydrochloride, hydrobromide, hydroiodide, sulfate, phosphate, and the like.

The alkali metal salts include sodium salt and potassium salt.

The alkaline earth metal salts include calcium salt and magnesium salt.

In addition, salts with organic bases include salts with ammonia, methylamine, triethylamine, N-methylmorpholine, and the like.

Note that, in Description and Claims, the compound represented by general formula (I) or a pharmaceutically acceptable salt thereof also includes those in the forms of hydrates and solvates.

### Applications

Monoacylglycerol acyltransferase (MGAT) is an enzyme for acylation of 2-monoacylglycerols (MGs) to diacylglycerols (DGs). Then, triacylglycerols (TGs) are re-synthesized from the DGs by diacylglycerol acyltransferase (DGAT), and the re-synthesized TGs are accumulated in the liver and tissues such as adipose tissue.

Accordingly, the compound represented by general formula (I) of the present invention or a pharmaceutically acceptable salt thereof which is capable of remarkably inhibiting the activity of the monoacylglycerol acyltransferase is useful as an MGAT inhibitor for inhibiting the activity of the monoacylglycerol acyltransferase (MGAT), especially, as a monoacylglycerol acyltransferase 2 (MGAT2) inhibitor.

In addition, the inhibition of the monoacylglycerol acyltransferase activity leads to the inhibition of the re-synthesis of DGs and TGs from MGs, and consequently, the absorption of fat into the body, especially, the liver and the adipose tissue is inhibited. Accordingly, the compound represented by general formula (I) of the present invention or a pharmaceutically acceptable salt thereof is useful as a fat absorption inhibitor.

Moreover, the compound represented by general formula (I) of the present invention or a pharmaceutically acceptable salt thereof which inhibits the MGAT activity is useful as an agent for treating and/or preventing a lipid metabolism disorder, or an agent for treating and/or preventing adiposity. Likewise, the compound represented by general formula (I) of the present invention or a pharmaceutically acceptable salt thereof is useful as a pharmaceutical composition, especially, as a pharmaceutical composition for treating and/or preventing a lipid metabolism disorder, or as a pharmaceutical composition for treating and/or preventing adiposity.

Examples of the lipid metabolism disorder include hypercholesterolemia and hypertriglyceridemia.

In each of the MGAT inhibitor, the fat absorption inhibitor, the agent for treating and/or preventing a lipid metabolism disorder, the agent for treating and/or preventing adiposity, and the pharmaceutical composition of the present invention, the compound represented by general formula (I) or a pharmaceutically acceptable salt thereof may be used alone, or may be used in the form of a composition comprising the compound represented by general formula (I) or a pharmaceutically acceptable salt thereof as an active ingredient.

For the use in the form of a composition, the composition may contain, for example, carriers (preferably, pharmaceutically or physiologically acceptable solid or liquid carriers), additives, and the like. Moreover, if necessary, a stabilizer, a wetting agent, an emulsifier, a binder, a tonicity adjusting agent, and the like may be added, as appropriate.

Examples of the carriers include glucose, lactose, sucrose, starch, mannitol, dextrin, fatty acid glyceride, polyethylene glycol, hydroxyethyl starch, ethylene glycol, polyoxyethylene sorbitan fatty acid ester, gelatin, albumin, amino acid, water, physiological saline, and the like.

The additives are not particularly limited, as long as the additives are commonly used for their individual purposes. Specific examples of the additives include flavors, saccharides, sweeteners, dietary fibers, vitamins, amino acids such as sodium glutamate (MSG), nucleic acids such as inosine monophosphate (IMP), inorganic salts such as sodium chloride, water, and the like.

### Dosage Form

The MGAT inhibitor, the fat absorption inhibitor, the agent for treating and/or preventing a lipid metabolism disorder, the agent for treating and/or preventing adiposity, and the pharmaceutical composition of the present invention can be used in orally administrable forms such as dry powders, pastes, and solutions, without any limitations on physical properties.

Examples of such orally administrable forms include tablets (including sugar-coated tablets, film-coated tablets, sublingual tablets, and orally disintegrating tablets), capsules (including soft capsules and microcapsules), granules, powders, troches, syrups, emulsions, suspensions, films (for example, orally disintegrating films), freeze-dried agents, and the like.

The MGAT inhibitor, the fat absorption inhibitor, the agent for treating and/or preventing a lipid metabolism disorder, the agent for treating and/or preventing adiposity, and the pharmaceutical composition of the present invention can be used also in the forms of parenteral agents such as injections (for example, subcutaneous injections, intravenous injections, intramuscular injections, intraperitoneal injections, and infusions), external agents (for example, transdermal preparations and ointments), suppositories (for example, rectal suppositories and vaginal suppositories), pellets, nasal agent, pulmonary preparations (inhalants), ophthalmic solutions, and the like.

These preparations can be safely administered orally or parenterally (for example, by local, rectal, or intravenous administration). These preparations may be rapid-release preparations or controlled-release preparations such as sustained-release preparations (for example, sustained-release microcapsules). These preparations can be prepared by ordinary pharmaceutical methods.

Moreover, the compound represented by general formula (I) or a pharmaceutically acceptable salt thereof can be used also in the forms accommodated in granules, tablets, gelatin capsules, and the like used for supplements and the like.

When the MGAT inhibitor, the agent for treating a lipid metabolism disorder, the agent for treating and/or preventing adiposity, the agent for inhibiting and/or preventing fat absorption, or the pharmaceutical composition of the present invention is administered to a human, the amount of the compound represented by general formula (I) of the present invention or a pharmaceutically acceptable salt thereof administered only needs to be an amount (effective amount) effective for inhibiting the activity of MGAT. The amount varies depending on the subject of administration, the administration route, the target disease, the symptom, and the like; however, for example, when orally administered to a human, the compound represented by general formula (I) of the present invention or a pharmaceutically acceptable salt thereof may be administered in an amount of approximately 0.3 mg/kg/day to approximately g/kg/day, preferably approximately 1 mg/kg/day to approximately 500 mg/kg/day, more preferably approximately 15 mg/kg/day to 200 mg/kg/day, and further preferably approximately 20 mg/kg/day to 50 mg/kg/day. The amount may be administered once per day, or may be administered one to five times per day, preferably, one to three times per day, or may be administered at intervals of once per 2, 3, 4, 5, 6, 7, or more days.

To reinforce the effect of the MGAT inhibitor of the present invention, the MGAT inhibitor may be used in combination with other anti-obesity agents, agents for treating diabetes mellitus, agents for treating hyperlipidemia, and the like.

When the MGAT inhibitor, the fat absorption inhibitor, the agent for treating and/or preventing a lipid metabolism disorder, the agent for treating and/or preventing adiposity, or the pharmaceutical composition of the present invention is used in combination with an anti-obesity agent, an agent for treating diabetes mellitus, or an agent for treating hyperlipidemia, the timing of the administration of the two is not particularly limited, and the two may be administered simultaneously to the subject of administration, or administered separately at a time interval.

### Production Methods

Methods for producing a compound represented by general formula (I) of the present invention are described below. The compound of the present invention can be produced by, for example, the following production method A, B, C, or D.

### Production Method A

Of the compounds represented by general formula (I), a Compound (A8 shown below) in which Y is —S— can be produced by, for example, the following production method.

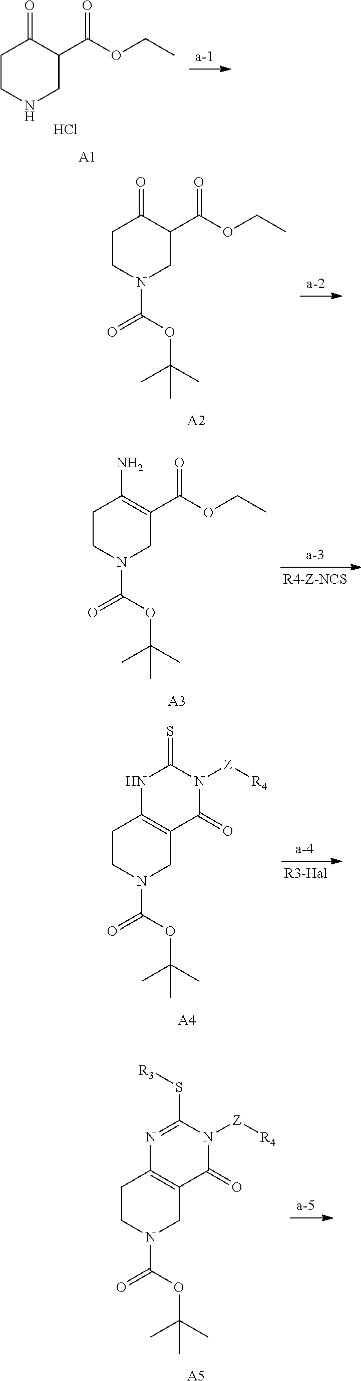

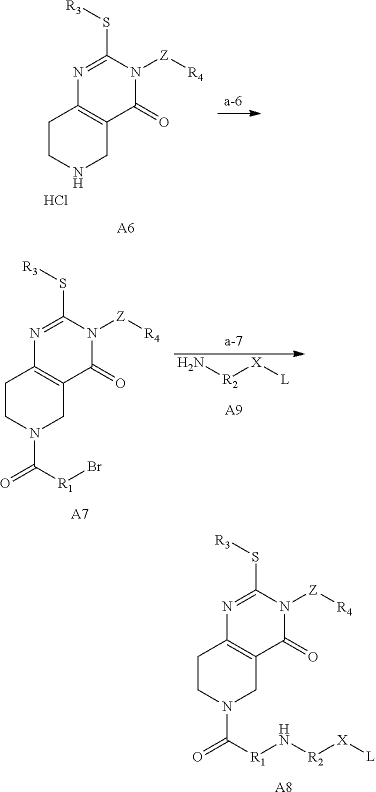

[Step a-1]

In this step, Compound (A1) is reacted with Boc₂O (di-tert-butyl dicarbonate) in the presence of a suitable solvent and a suitable amine, to obtain Compound (A2). The solvent is preferably dichloromethane, and the amine is preferably triethylamine. After the reaction, purification is carried out by extraction with ethyl acetate and water.

[Step a-2]

In this step, Compound (A2) is heated in the presence of ammonia water to obtain (A3). After the reaction, purification is carried out by extraction with ethyl acetate and water.

[Step a-3]

In this step, a corresponding isothiocyanate is added to Compound (A3) in the presence of a suitable solvent and a suitable amine, followed by heating to obtain (A4). The reaction liquid is concentrated, and then purified by crystallization.

[Step a-4]

In this step, a corresponding alkyl halide (R₃—Hal) is added to Compound (A4) in the presence of a suitable solvent and a suitable amine, followed by stirring to obtain (A5). The reaction liquid is extracted, concentrated, and purified by column chromatography, or the reaction liquid is concentrated, and then purified by crystallization.

[Step a-5]

In this step, a protective group is removed from Compound (A5) for deprotection in the presence of a suitable solvent and a suitable acid catalyst to obtain (A6). The reaction liquid is concentrated, and purified by crystallization.

[Step a-6]

In this step, a corresponding acyl bromide is added to Compound (A6) in the presence of a suitable solvent and a suitable amine, to obtain (A7). The reaction liquid is extracted, and purified by column chromatography.

[Step a-7]

In this step, Amine (A9) is added to Compound (A7) in the presence of a suitable solvent and a suitable amine, to obtain (A8). The reaction liquid is extracted, and purified by column chromatography.

### Production Method B

Of the compounds represented by general formula (I), a compound (B6 shown below) in which Y is —NH— can be produced by, for example, the following production method.

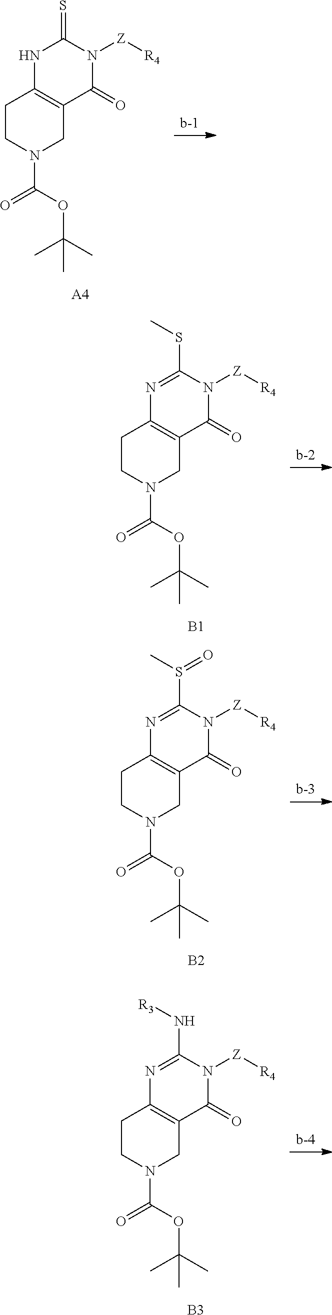

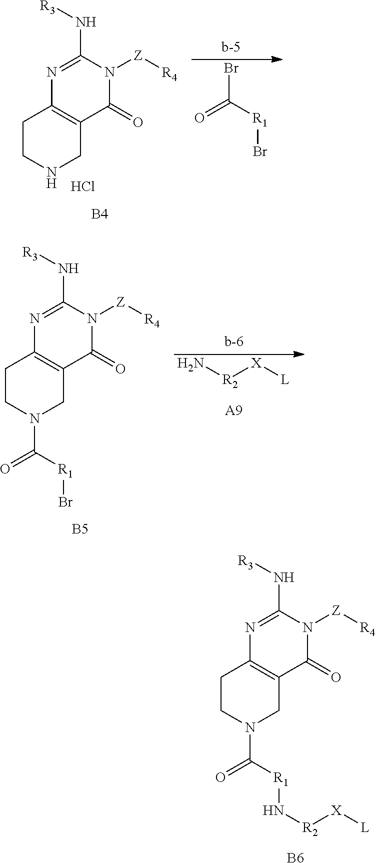

[Step b-1]

In this step, Compound (A4) is reacted with MeI (methyl iodide) in the presence of a suitable solvent and a suitable base, to obtain Compound (B1). The solvent is preferably DMF (N,N-dimethylformamide), and the base is preferably potassium carbonate. After the reaction, purification is carried out by extraction with ethyl acetate and water.

[Step b-2]

In this step, Compound (B1) is oxidized with m-CPBA (m-chloroperoxybenzoic acid) in the presence of a suitable solvent, to obtain (B2). After the reaction, purification is carried out by extraction with ethyl acetate and water.

[Step b-3]

In this step, a corresponding amine is added to Compound (B2) in the presence of a suitable solvent and a suitable base, to obtain (B3). The reaction liquid is concentrated, and then purified by column chromatography.

[Step b-4]

In this step, a protective group is removed from Compound (B3) for deprotection by using a suitable solvent and a suitable acid catalyst, to obtain (B4). The reaction liquid is concentrated, and purified by crystallization.

[Step b-5]

In this step, a corresponding acyl bromide is added to Compound (B4) in the presence of a suitable solvent and a suitable base, to obtain (B5). The reaction liquid is extracted, and purified by column chromatography.

[Step b-6]

In this step, Amine (A9) is added to Compound (B5) in the presence of a suitable solvent and a suitable base, to obtain (B6). The reaction liquid is extracted, and purified by column chromatography.

### Production Method C

Of the compounds represented by general formula (I), Compound (C2) in which Y is —S— and R₁ is —CH₂CH₂— can be produced by, for example, the following production method.

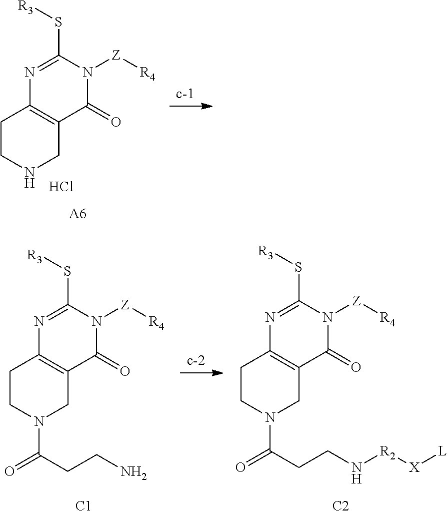

[Step c-1]

In this step, (C1) is obtained as follows. Specifically, in the presence of a suitable solvent and a suitable base, WSC (water-soluble carbodiimide), N-Boc-beta-alanine are added to Compound (A6), followed by stirring at room temperature. Subsequently, after concentration under reduced pressure, trifluoroacetic acid is added. Then, after concentration under reduced pressure, purification is carried out by using a reversed-phase preparative column to obtain (C1).

[Step c-2]

In this step, (C2) is obtained as follows. Specifically, in the presence of a suitable solvent and a suitable acid catalyst, a suitable aldehyde and sodium cyanoborohydride are added to Compound (C1), followed by stirring. After concentration under reduced pressure, purification is carried out by column chromatography to obtain (C2).

### Production Method D

Of the compounds represented by general formula (I), a compound in which R₁ is an ethylene group (—CH₂CH₂—) can also be produced by, for example, the following production method, in addition to the above-described production methods.

[Step d-1]

In this step, acryloyl chloride is added to and reacted with a compound represented by the following general formula (II) in the presence of a suitable solvent and a suitable base, to obtain a compound represented by the following general formula (III).

Examples of the solvent include dichloromethane and tetrahydrofuran. Examples of the base include diisopropylethylamine and triethylamine.

[Step d-2]

In this step, an alkylated amine represented by Formula L-X—R₂—NH₂, where L, X, and R₂ are the same as L, X, and R₂ in general formula (I), respectively, is added to and reacted with the obtained compound represented by general formula (III) in the presence of a suitable solvent and a suitable base, to obtain the compound represented by general formula (I).

Examples of the solvent include tetrahydrofuran and 1,4-dioxane. Examples of the base include diazabicycloundecene and diisopropylethylamine.

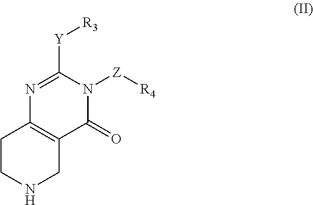

In the formula, Y, Z, R₃, and R₄ are the same as Y, Z, R₃, and R₄ in general formula (I), respectively.

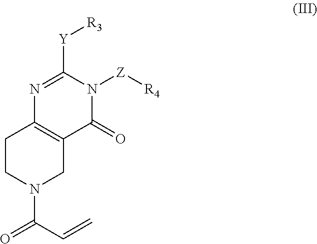

In the formula, Y, Z, R₃, and R₄ are the same as Y, Z, R₃, and R₄ in general formula (I), respectively.

Of the compounds represented by general formula (I), Compound (D2) in which Y is —S—, and R₁ is —CH₂CH₂— can be produced by, for example, the following production method.

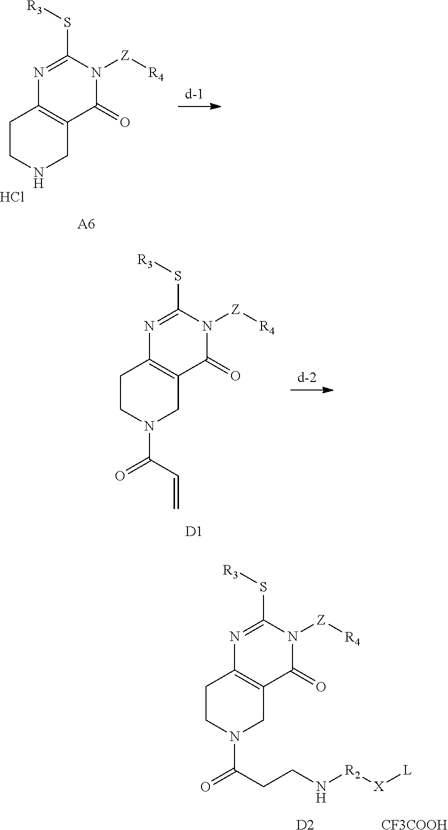

[Step d′-1]

In this step, (D1) is obtained as follows. Specifically, in the presence of a suitable solvent and a suitable base, acryloyl chloride is added to Compound (A6), followed by stirring at room temperature. After the reaction, extraction and drying are carried out. Then, after concentration under reduced pressure, purification is carried out by using a preparative column to obtain (D1).

[Step d′-2]

In this step, (D2) is obtained as follows. Specifically, in the presence of a suitable solvent and a suitable base, a suitable alkylated amine is added to Compound (D1), followed by stirring under heating at 80° C. After concentration under reduced pressure, purification is carried out by using a reversed-phase preparative column to obtain (D2).

### EXAMPLES

Hereinafter, the present invention is described specifically based on Examples. However, the present invention is not limited to the contents described in these Examples.

### Reference Example 1

### Synthesis of 2-Cylobutylsulfanyl-3-phenyl-5,6,7,8-tetrahydropyrido[4,3-d]pyrimidin-4-one Hydrochloride (Compound of the Following Formula (VI))

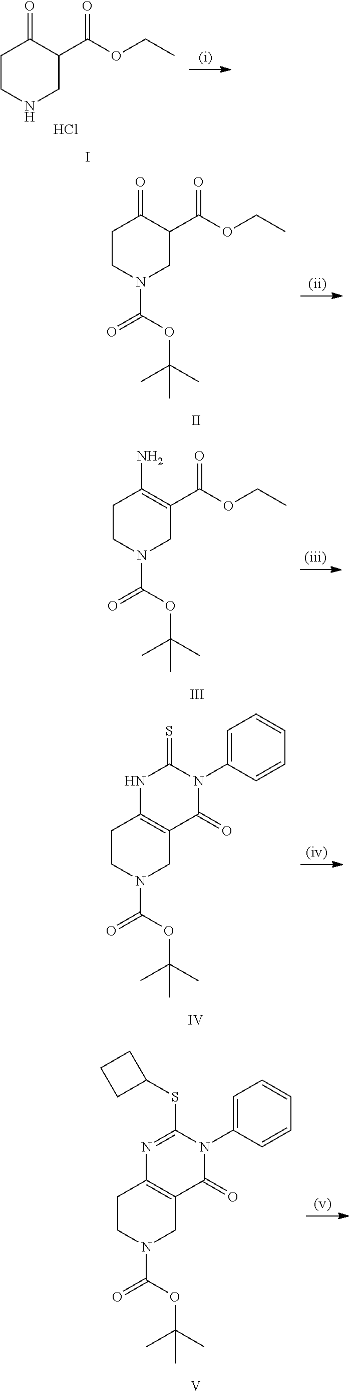

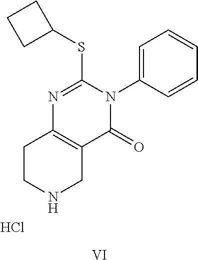 [SMILES: O=c1c2c(nc(SC3CCC3)n1-c1ccccc1)CCNC2 ; Cl ; "VI"]

### (Step (i))

Compound I (10.0 g) was dissolved in dichloromethane (200 mL), and triethylamine (20 mL) was added. Subsequently, Boc₂O (10.6 g) was added, followed by stirring at room temperature for 20 hours. To the reaction liquid, saturated aqueous sodium chloride was added, followed by extraction with dichloromethane three times. The organic layer was dried over anhydrous sodium sulfate, and concentrated under reduced pressure. Without purification, the product was used in Step (ii), as it was.

(Step (ii))

To Compound II, 28% ammonia water (130 mL) was added, followed by stirring at 80° C. for 2.5 hours. To the reaction liquid, saturated aqueous sodium chloride was added, followed by extraction with dichloromethane three times. The organic layer was dried over anhydrous sodium sulfate, and concentrated under reduced pressure. Purification was carried out by using a silica gel column (ethyl acetate:hexane=40/60) to obtain

Compound III (9.7 g, 750).

¹H NMR (400 MHz, CDCl₃) δ 4.10-4.20 (2H, m), 4.07 (2H, brs), 3.72 (1H, t, J=4.7 Hz), 3.51 (2H, t, J=4.4 Hz), 2.44 (1H, t, J=4.7 Hz), 2.28 (2H, t, J=4.3 Hz), 1.47 (9H, 5), 1.25 (3H, t, J=5.3 Hz). MS: 271 [M+14]+

(Step (iii))

Compound III (9.7 g) was dissolved in pyridine (49 mL), and phenyl isothiocyanate (8.6 mL) was added, followed by stirring at 90° C. for 15 hours. The reaction liquid was concentrated under reduced pressure, and dissolved in toluene. Crystallization by adding diethyl ether yielded Compound IV (8.2 g, 64%).

¹H. NMR (400 MHz, DMSO-d₆) δ 12.74 (1H, brs), 7.36-7.48 (3H, tri), 7.16-7.18 (2H, m), 4.06 (2H, brs), 3.57 (2H, t, J=4.2 Hz), 2.55 (2H, brs), 1.42 (9H, s). MS: 360 [M+H]+

(Step (iv))

To a DMF (100 ml) solution of Compound IV (10.0 g), cyclobutyl bromide (2.9 ml) and DBU (diazabicycloundecene) (4.6 ml) were added, followed by stirring at 40° C. for 2 days. After the reaction, aqueous sodium chloride was added, followed by extraction with ethyl acetate three times. The organic layer was dried over anhydrous sodium sulfate, and then concentrated under reduced pressure. The obtained compound was used in the next reaction, without purification.

### (Step (v))

To a methanol (100 ml) solution of Compound V, 30 ml of 4 N—HCl/dioxane was added, followed by stirring at 0° C. for 6 hours. After the reaction, the solution was concentrated under reduced pressure. The obtained solid was crystallized from methanol-ethyl acetate to obtain Compound VI (5.9 g).

¹H NMR (400 MHz, DMSO-d₆) δ 8.80 (s, 2H), 7.61-7.51 (m, 3H), 7.36-7.27 (m, 4H), 7.26-7.17 (m, 3H), 4.32 (s, 1H), 4.27-4.12 (m, 4H), 3.81 (t, J=5.8 Hz, 1H), 3.65 (t, J=5.7 Hz, 1H), 2.98-2.86 (m, 2H), 2.80-2.73 (m, 1H), 2.70-2.59 (m, 2H), 2.44-2.28 (m, 3H), 2.04-1.86 (m, 6H). MS: 314 [M+H]+

### Reference Example 2

### Synthesis of 2-(Cyclobutylamino)-3-(2-furylmethyl)-5,6,7,8-tetrahydropyrido[4,3-d]pyrimidin-4-one Hydrochloride (Compound of the Following Formula (XI))

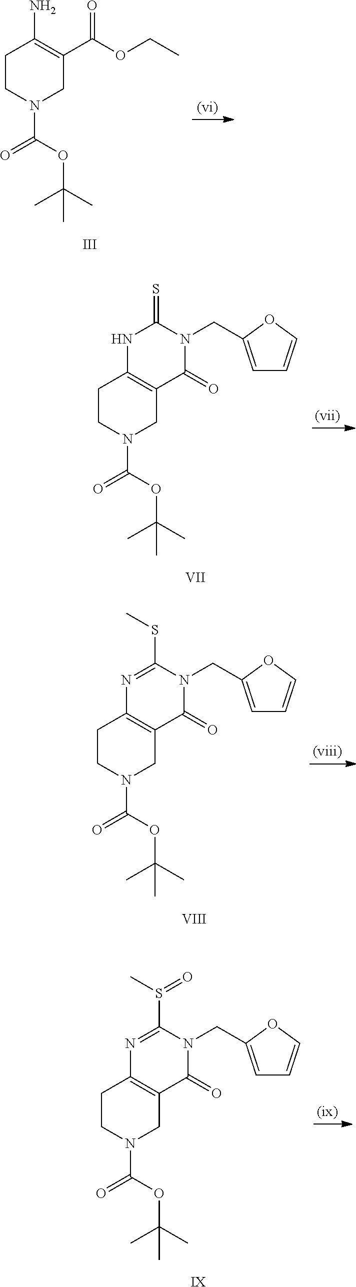

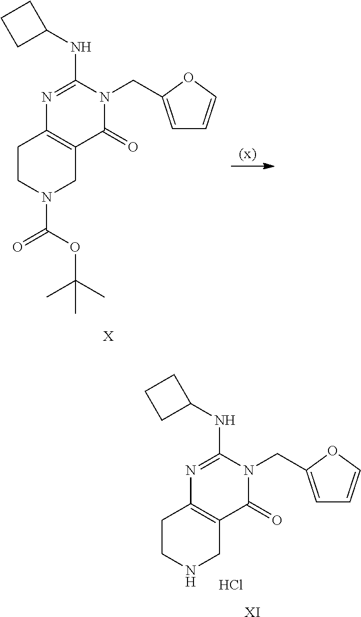

(Step (vi))

Compound III (2.0 g) was dissolved in pyridine (40 mL), and 2-furylmethyl isothiocyanate (2.1 g) was added, followed by stirring at 90° C. for 15 hours. The reaction liquid was concentrated under reduced pressure, and purified by silica gel column chromatography (petroleum ether:ethyl acetate=5:1) to obtain Compound VII (1.8 g, 670).

¹H NMR (400 MHz, DMSO-d₆) δ 12.73 (1H, s), 7.55 (1H, d, J=1.2 Hz), 6.37-6.39 (1H, m), 6.33-6.34 (1H, m), 5.51 (2H, s), 4.05 (2H, s), 3.52 (2H, t, J=5.2 Hz), 2.50-2.51 (2H, m), 1.41 (9H, s).

(Step (vii))

Compound VII (1.0 g) was dissolved in DMF (20 mL), and DBU (629 mg) was added. Under ice-cooling, methyl iodide (587 mg) was added, followed by stirring for 12 hours. To the reaction liquid, water (60 mL) was added, followed by extraction with ethyl acetate. The organic layer was dried over anhydrous sodium sulfate, and concentrated under reduced pressure. Then, purification was carried out by silica gel column chromatography (petroleum ether:ethyl acetate=10:1) to obtain Compound VIII (1.0 g, 96%).

(Step (viii))

Compound VIII (1.0 g), sodium acetate (656 mg), and magnesium sulfate (387 mg) were dissolved in dichloromethane. The mixture was purged with argon, and then cooled to −70° C. A dichloromethane (10 mL) solution of 85% mCPBA (1.62 g) was added, followed by stirring at −50° C. for 2 hours. To the reaction solution, 10% sodium thiosulfate was added, and the temperature was returned to room temperature. After extraction with dichloromethane, the organic layer was washed with an aqueous sodium hydrogen carbonate solution, and with aqueous sodium chloride. The organic layer was dried over anhydrous sodium sulfate, and concentrated under reduced pressure. Then, purification was carried out by silica gel column chromatography (petroleum ether:ethyl acetate=5:1) to obtain Compound IX (400 mg, 38%).

¹H NMR (400 MHz, CDCl3) δ 7.37 (1H, s), 6.50 (1H, s), 6.36 (1H, s), 5.70-5.80 (1H, m), 5.35-5.39 (1H, m), 4.38-4.42 (2H, m), 3.65-3.73 (2H, m), 2.92 (3H, s), 2.77-2.78 (2H, m), 1.48 (9H, s).

(Step (ix))

Compound IX (400 mg) was dissolved in dioxane (10 mL), and DIEA (N,N-diisopropylethylamine) (144 mg), cyclobutylamine (145 mg), and DMAP (N,N-dimethyl-4-aminopyridine) (13 mg) were added, followed by stirring at 50° C. for 12 hours. After cooling to room temperature, water was added. After extraction with ethyl acetate, the organic layer was washed with aqueous sodium chloride, and dried over anhydrous sodium sulfate. After concentration under reduced pressure, purification was carried out by silica gel column chromatography (petroleum ether:ethyl acetate=10:1) to obtain Compound X (200 mg, 49%). MS: 401 [M+H]+

### (Step (x))

Compound X (200 mg) was dissolved in ethyl acetate (5 mL), and 3 M-HCl/EtOAc (1 mL) was added at 0° C. After stirring at room temperature for 1 hour, the mixture was concentrated under reduced pressure to obtain Compound XI (143 mg, 850).

¹H NMR (400 MHz, DMSO-d₆) δ 9.57 (2H, s), 7.78 (1H, s), 7.60 (1H, d, J=0.8 Hz), 6.60-6.44 (2H, m), 5.32 (2H, s), 4.51-4.45 (1H, m), 3.76 (2H, s), 3.29 (2H, s), 2.69-2.71 (2H, m), 2.20-2.26 (2H, m), 2.03-2.14 (2H, m), 1.59-1.72 (2H, m). MS: 301 [M+14]+

### Example 1

### Synthesis of 2-Cyclobutylsulfanyl-3-phenyl-6-[2-[[4-(trifluoromethyl)phenyl]methylamino]acetyl]-7,8-dihydro-5H-pyrido[4,3-d]pyrimidin-4-one Trifluoroacetate (Compound of the Following Formula (XIV)

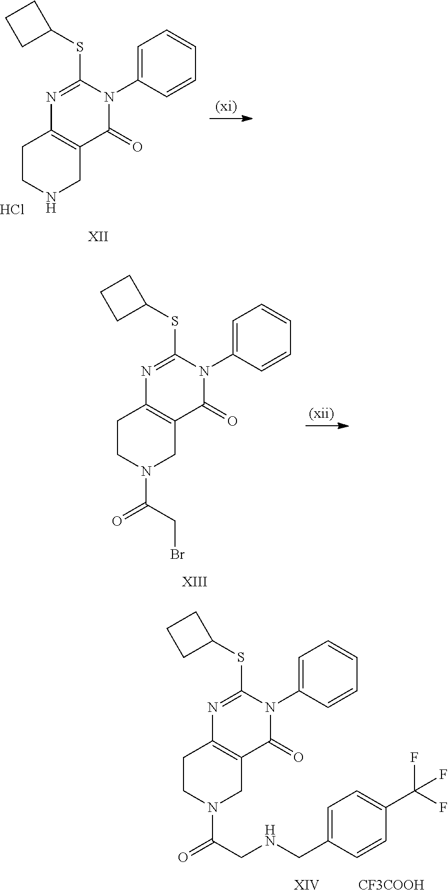

(Step (xi))

To an acetonitrile (1 ml) solution of Compound XII (the same as Compound VI of Reference Example 1) (20 mg), DIPEA (N,N-diisopropylethylamine) (0.020 ml) and bromoacetyl bromide (0.005 ml) were added, followed by stirring at room temperature for 20 minutes. The mixture was used, as it was, for the next reaction.

(Step (xii))

To the reaction solution obtained in Step (xi), 4-trifluoromethylbenzylamine (0.010 ml) and DIPEA (0.010 ml) were added, followed by stirring at room temperature for 1 hour. The reaction liquid was purified by using a reversed-phase preparative column to obtain the target Compound XIV (5.4 mg).

### Examples 2 to 9

Compounds of Examples 2 to 9 shown in Table 1 below were synthesized according to the method in Example 1.

### Example 10

### Synthesis of 6-[3-(Benzylamino)propanoyl]-2-cyclobutylsulfanyl-3-phenyl-7,8-dihydro-5H-pyrido[4,3-d]pyrimidin-4-one Trifluoroacetate (Compound of the Following Formula (XVII))

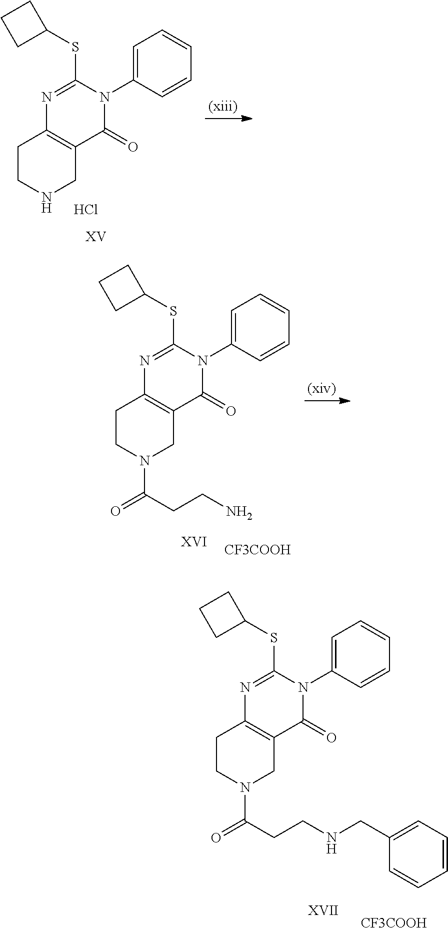

(Step (xiii))

To a dichloromethane (0.5 ml) solution of Compound XV (which was the same as Compound VI of Reference Example 1 described above) (190 mg), N-Boc-beta-alanine (134 mg), WSC.HCl (140 mg), and DIPEA (0.28 ml) were added, followed by stirring at room temperature for 5 hours. After concentration under reduced pressure, 2 ml of TFA (trifluoroacetic acid) was added, followed by stirring at room temperature for 2 hours. After concentration under reduced pressure, purification was carried out by using a reversed-phase preparative column to obtain 160 mg of Compound XVI.

(Step (xiv))

Compound XVI (102 mg) was dissolved in dichloromethane (0.6 ml), methanol (0.3 ml), and acetic acid (50 μl). Benzaldehyde (31 μl) and sodium cyanoborohydride (19 mg) were added, followed by stirring at room temperature for 4 hours. After concentration under reduced pressure, purification was carried out by using a reversed-phase preparative column to obtain Compound XVII (45 mg).

### Examples 11 to 24

Compounds of Examples 11 to 24 shown in Table 1 below were synthesized according to the method in Example 10.

TABLE 1

| Compound | Structural | MASS | | Compound Name |
|---|---|---|---|---|
| No. | formula | (ESI) | NMR | (IUPAC) |
| Ex. 1 | 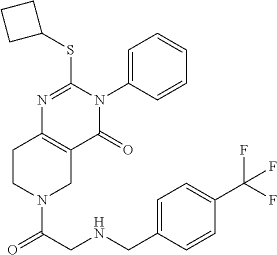 [SMILES: O=C(CNCc1ccc(C(F)(F)F)cc1)N1CCc2nc(SC3CCC3)n(-c3ccccc3)c(=O)c2C1] CF3COOH | 529 (M + H)+ | ¹H NMR (400 MHz, DMSO-d₆) δ 9.35 (s, 2H), 7.84 (dd, J = 8.3, 3.6 Hz, 2H), 7.74 (t, J = 7.5 Hz, 2H), 7.61-7.50 (m, 3H), 7.36-7.26 (m, 2H), 4.32 (s, 1H), 4.29-4.12 (m, 6H), 3.81 (t, J = 5.5 Hz, 1H), 3.64 (t, J = 5.7 Hz, 1H), 2.77 (t, J = 5.7 Hz, 1H), 2.64 (d, J = 5.8 Hz, 1H), 2.44-2.35 (m, 2H), 2.04- 1.86 (m, 4H). | 2-cyclobutyl- sulfanyl-3- phenyl-6-[2-[[4- (trifluoromethyl) phenyl] methylamino] acetyl]-7,8-dihydro- 5H-pyrido [4,3-d]pyrimidin- 4-one trifluoroacetate |
| Ex. 2 | 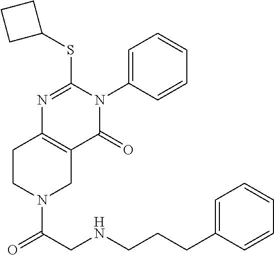 [SMILES: O=C(CNCCCc1ccccc1)N1CCc2nc(SC3CCC3)n(-c3ccccc3)c(=O)c2C1] CF3COOH | 489 (M + H)+ | ¹H NMR (400 MHz, DMSO-d₆) δ 8.80 (s, 2H), 7.61-7.51 (m, 3H), 7.36-7.27 (m, 4H), 7.26-7.17 (m, 3H), 4.32 (s, 1H), 4.27-4.12 (m, 4H), 3.81 (t, J = 5.8 Hz, 1H), 3.65 (t, J = 5.7 Hz, 1H), 2.98-2.86 (m, 2H), 2.80- 2.73 (m, 1H), 2.70-2.59 (m, 2H), 2.44-2.28 (m, 3H), 2.04-1.86 (m,6H).= | 2-cyclobutyl- sulfanyl-3- phenyl-6-[2-(3- phenylpropyl- amino)acetyl]-7, 8-dihydro-5H- pyrido[4,3-d] pyrimidin-4- one trifluoroacetate |
| Ex. 3 | 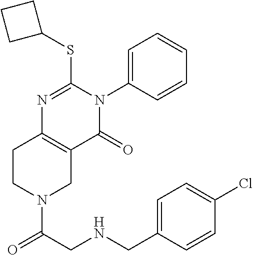 [SMILES: O=C(CNCc1ccc(Cl)cc1)N1CCc2nc(SC3CCC3)n(-c3ccccc3)c(=O)c2C1] CF3COOH | 495 (M + H)+ | ¹H NMR (400 MHz, DMSO-d₆) δ 9.24 (s, 2H), 7.63-7.48 (m, 7H), 7.38-7.26 (m, 2H), 4.32 (s, 1H), 4.25-4.10 (m, 6H), 3.80 (t, J = 5.9 Hz, 1H), 3.63 (t, J = 5.7 Hz, 1H), 2.76 (t, J = 6.2 Hz, 1H), 2.64 (t, J = 6.2 Hz, 1H), 2.44-2.35 (m, 2H), 2.04- 1.87 (m, 4H). | 6-[2-[(4-chloro- phenyl) methylamino]acetyl]- 2-cyclobutyl- sulfanyl-3- phenyl-7,8- dihydro-5H- pyrido[4,3-d] pyrimidin-4-one trifluoroacetate |
| Ex. 4 | 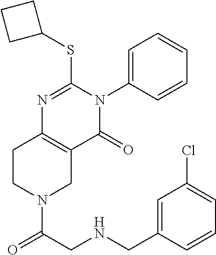 [SMILES: O=C(CNCc1cccc(Cl)c1)N1CCc2nc(SC3CCC3)n(-c3ccccc3)c(=O)c2C1] CF3COOH | 495 (M + H)+ | ¹H NMR (400 MHz, DMSO-d₆) δ 9.28 (s, 2H), 7.66-7.42 (m, 7H), 7.37-7.25 (m, 2H), 4.32 (s, 1H), 4.25-4.12 (m, 6H), 3.80 (d, J = 5.8 Hz, 1H), 3.63 (t, J = 5.9 Hz, 1H), 2.76 (t, J = 5.6 Hz, 1H), 2.64 (t, J = 4.9 Hz, 1H), 2.44-2.35 (m, 2H), 2.03- 1.88 (m, 4H). | 6-[2-[(3-chloro- phenyl) methylamino]acetyl]- 2-cyclobutyl- sulfanyl-3- phenyl-7,8-dihydro- 5H-pyrido 4,3-[d]pyrimidin- 4-one trifluoroacetate |
| Ex. 5 | 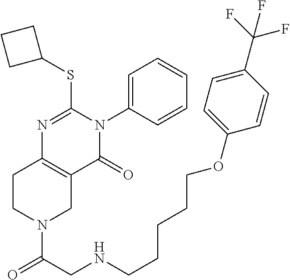 [SMILES: O=C(CNCCCCCOc1ccc(C(F)(F)F)cc1)N1CCc2nc(SC3CCC3)n(-c3ccccc3)c(=O)c2C1] CF3COOH | 601 (M + H)+ | 1H NMR (400 MHz, DMSO-d6) δ 8.78 (s, 2H), 7.65 (dd, J = 8.6, 3.8 Hz, 2H), 7.61- 7.49 (m, 3H), 7.37-7.26 (m, 2H), 7.11 (dd, J = 8.5, 4.4 Hz, 2H), 4.38-4.12 (m, 5H), 4.06 (dd, J = 6.2, 6.2 Hz, 2H), 3.82 (t, J = 5.8 Hz, 1H), 3.66 (t, J = 5.8 Hz, 1H), 3.02-2.85 (m, 2H), 2.83-2.74 (m, 1H), 2.69- 2.62 (m, 1H), 2.44-2.31 (m, 2H), 2.05-1.87 (m, 4H), 1.83-1.63 (m, 4H), 1.55- 1.40 (m, 2H). | 2-cyclobutyl- sulfanyl-3- phenyl-6-[2-[5- [4-(trifluoromethyl) phenoxy] pentylamino] acetyl]-7,8- dihydro-5H- pyrido[4,3-d] pyrimidin-4-one trifluoroacetate |
| Ex. 6 | 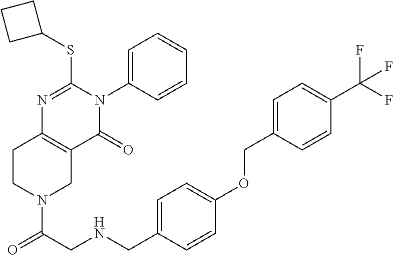 [SMILES: O=C(CNCc1ccc(OCc2ccc(C(F)(F)F)cc2)cc1)N1CCc2nc(SC3CCC3)n(-c3ccccc3)c(=O)c2C1] CF3COOH | 635 (M + H)+ | — | 2-cyclobutyl- sulfanyl-3- phenyl-6-[2-[[4- [[4-(trifluoro- methyl)phenyl] methoxy]phenyl] methylamino]acetyl]- 7,8-dihydro-5H- pyrido[4,3-d] pyrimidin-4- one hydrate trifluoroacetate |
| Ex. 7 | 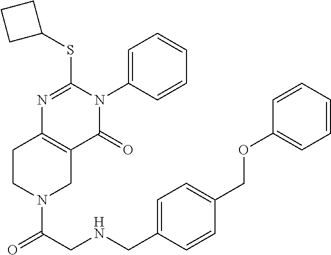 [SMILES: O=C(CNCc1ccc(COc2ccccc2)cc1)N1CCc2nc(SC3CCC3)n(-c3ccccc3)c(=O)c2C1] CF3COOH | 567 (M + H)+ | — | 2-cyclobutyl- sulfanyl-6-[2- [[4-(phenoxy- methyl)phenyl] methylamino] acetyl]-3-phenyl- 7,8-dihydro- 5H-pyrido[4, 3-d]pyrimidin-4-one trifluoroacetate |
| Ex. 8 | 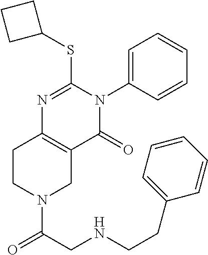 [SMILES: O=C(CNCCc1ccccc1)N1CCc2nc(SC3CCC3)n(-c3ccccc3)c(=O)c2C1] CF3COOH | 475 (M + H)+ | ¹H NMR (400 MHz, DMSO-d₆) δ 8.95 (s, 2H), 7.62-7.50 (m, 3H), 7.40-7.20 (m, 7H), 4.34 (s, 1H), 4.30-4.14 (m, 4H), 3.82 (t, J = 5.7 Hz, 1H), 3.66 (t, J = 5.7 Hz, 1H), 3.22-3.11 (m, 2H), 2.98 (dd, J = 16.6, 8.9 Hz, 2H), 2.78 (t, J = 5.8 Hz, 1H), 2.69- 2.64 (m, 1H), 2.44-2.35 (m, 2H), 2.02-1.87 (m, 4H). | 2-cyclobutyl- sulfanyl-6-[2- (phenylethylamino) acetyl]-3- phenyl-7,8- dihydro-5H- pyrido[4,3-d] pyrimidin-4-one trifluoroacetate |
| Ex. 9 | 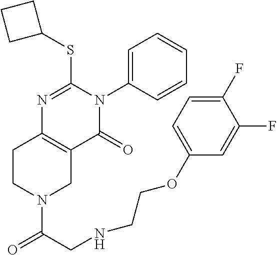 [SMILES: O=C(CNCCOc1ccc(F)c(F)c1)N1CCc2nc(SC3CCC3)n(-c3ccccc3)c(=O)c2C1] CF3COOH | 527 (M + H)+ | ¹H NMR (400 MHz, DMSO-d₆) δ 9.05 (s, 2H), 7.63-7.52 (m, 3H), 7.40 (ddd, J = 20.0, 9.4, 5.1 Hz, 1H), 7.34- 7.28 (m, 2H), 7.16-7.07 (m, 1H), 6.87-6.78 (m, 1H), 4.32 (s, 1H), 4.31-4.23 (m, 5H), 4.23-4.13 (m, 1H), 3.81 (t, J = 5.9 Hz, 1H), 3.67 (t, J = 5.6 Hz, 1H), 3.30-3.26 (m, 2H), 2.78 (t, J = 5.5 Hz, 1H), 2.68-2.61 (m, 1H), 2.43-2.35 (m, 2H), 1.95 (dd, J = 13.1, 6.6 Hz, 4H). | 2-cyclobutyl- sulfanyl-6-[2- [2-(3,4- difluorophenoxy) ethylamino] acetyl]-3-phenyl- 7,8-dihydro-5H- pyrido[4,3- d]pyrimidin-4- one trifluoroacetate |
| Ex. 10 | 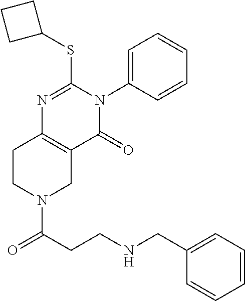 [SMILES: O=C(CCNCc1ccccc1)N1CCc2nc(SC3CCC3)n(-c3ccccc3)c(=O)c2C1] CF3COOH | 475 (M + H)+ | ¹H NMR (400 MHz, DMSO-d₆) δ 8.73 (br, 2H), 7.61- 7.39 (m, 8H), 7.35-7.27 (m, 2H), 4.29 (d, J = 13.5 Hz, 2H), 4.24-4.12 (m, 3H), 3.77 (t, J = 5.7 Hz, 1H), 3.70 (t, J = 5.7 Hz, 1H), 3.22- 3.08 (m, 2H), 2.91-2.82 (m, 2H), 2.74 (t, J = 5.7 Hz, 1H), 2.63 (t, J = 5.7 Hz, 1H), 2.44- 2.30 (m, 2H), 2.03-1.86 (m, 4H). | 6-[3-(benzyl- amino)propanoyl]- 2-cyclobutyl- sulfanyl-3- phenyl-7,8- dihydro-5H- pyrido[4,3-d] pyrimidin-4-one trifluoroacetate |
| Ex. 11 | 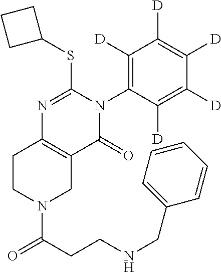 [SMILES: [2H]c1c([2H])c([2H])c(-n2c(SC3CCC3)nc3c(c2=O)CN(C(=O)CCNCc2ccccc2)CC3)c([2H])c1[2H]] CF3COOH | 480 (M + H)+ | 1H NMR (400 MHz, DMSO-d6) δ 8.70 (br, 2H), 7.55- 7.40 (m, 5H), 4.29 (d, J = 13.6 Hz, 2H), 4.25- 4.15 (m, 3H), 3.77 (t, J = 5.7 Hz, 1H), 3.70 (t, J = 5.7 Hz, 1H), 3.20-3.10 (m, 2H), 2.89-2.82 (m, 2H), 2.74 (t, J = 5.6 Hz, 1H), 2.63 (t, J = 5.6 Hz, 1H), 2.45-2.30 (m, 2H), 2.03-1.86 (m, 4H). | 6-[3-(benzyl- amino)propanoyl]- 2-cyclobutyl- sulfanyl-3- (2,3,4,5,6- pentadeuterio- phenyl)-7,8- dihydro-5H- pyrido[4,3-d] pyrimidin-4-one trifluoroacetate |
| Ex. 12 | 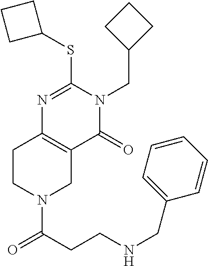 [SMILES: O=C(CCNCc1ccccc1)N1CCc2nc(SC3CCC3)n(CC3CCC3)c(=O)c2C1] CF3COOH | 467 (M + H)+ | 1H NMR (400 MHz, DMSO-d6) δ 8.69 (br, 2H), 7.52- 7.40 (m, 5H), 4.30-4.18 (m, 5H), 4.02-3.97 (m, 2H), 3.71 (t, J = 6.0 Hz, 1H), 3.65 (t, J = 6.0 Hz, 1H), 3.20- 3.08 (m, 2H), 2.86-2.80 (m, 2H), 2.78-2.64 (m, 2H), 2.58-2.43 (m, 1H), 2.15- 1.75 (m, 12H). | 6-[3-(benzyl- amino)propanoyl]- 3-(cyclo- butylmethyl)-2- cyclobutyl- sulfanyl-7,8- dihydro-4H- pyrido[4,3-d] pyrimidin-4-one trifluoroacetate |
| Ex. 13 | 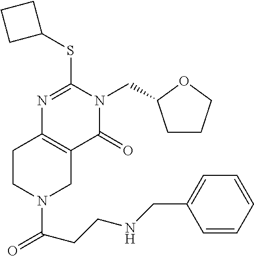 [SMILES: O=C(CCNCc1ccccc1)N1CCc2nc(SC3CCC3)n(C[C@H]3CCCO3)c(=O)c2C1] CF3COOH | 483 (M + H)+ | 1H NMR (400 MHz, DMSO-d6) δ 8.67 (br, 2H), 7.52- 7.40 (m, 5H), 4.31-4.16 (m, 5H), 4.03-3.60 (m, 7H), 3.18-3.10 (m, 2H), 2.86- 2.80 (m, 2H), 2.68-2.62 (m, 2H), 2.15-1.75 (m, 10H). | 6-[3-(benzyl- amino)propanoyl]- 2-cyclobutyl- sulfanyl-3- [[(2R)-tetra- hydrofuran-2-yl] methyl]-7,8- dihydro-5H- pyrido[4,3-d] pyrimidin-4-one trifluoroacetate |
| Ex. 14 | 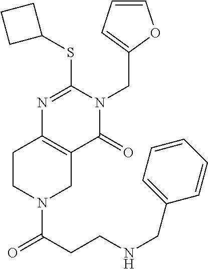 [SMILES: O=C(CCNCc1ccccc1)N1CCc2nc(SC3CCC3)n(Cc3ccco3)c(=O)c2C1] CF3COOH | 479 (M + H)+ | 1H NMR (400 MHz, DMSO-d6) δ 8.79 (s, 2H), 7.60 (t, J = 1.7 Hz, 1H), 7.56-7.48 (m, 2H), 7.48-7.36 (m, 3H), 6.47- 6.34 (m, 2H), 5.16 (d, J = 5.4 Hz, 2H), 4.36-4.13 (m, 5H), 3.72 (t, J = 5.8 Hz, 1H), 3.65 (t, J = 5.7 Hz, 1H), 3.21- 3.08 (m, 2H), 2.85 (t, J = 6.7 Hz, 2H), 2.66 (t, J = 5.2 Hz, 1H), 2.56 (t, J = 5.5 Hz, 1H), 2.50-2.40 (m, 2H), 2.17- 1.93 (m, 4H). | 6-[3-(benzyl- amino)propanoyl]- 2-cyclobutyl- sulfanyl-3- (2-furylmethyl)- 7,8- dihydro- 5H-pyrido[4, 3-d]pyrimidin- 4-one trifluoroacetate |
| Ex. 15 | 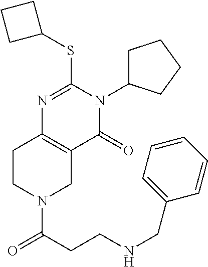 [SMILES: O=C(CCNCc1ccccc1)N1CCc2nc(SC3CCC3)n(C3CCCC3)c(=O)c2C1] CF3COOH | 467 (M + H)+ | 1H NMR (400 MHz, DMSO-d6) δ 8.79 (s, 2H), 7.56-7.48 (m, 2H), 7.48-7.38 (m, 3H), 4.73-4.58 (m, 1H), 4.31- 4.12 (m, 5H), 3.71 (t, J = 5.8 Hz, 1H), 3.64 (t, J = 5.7 Hz, 1H), 3.14 (s, 2H), 2.84 (m, J = 6.6 Hz, 2H), 2.63 (t, J = 5.6 Hz, 1H), 2.56-2.39 (m, 3H), 2.22-1.97 (m, 6H), 1.97- 1.75 (m, 4H), 1.66-1.49 (m, 2H). | 6-[3-(benzylamino) propanoyl]-2-cyclobutyl- sulfanyl-3- cyclopentyl-7, 8-dihydro-5H- pyrido[4,3-d] pyrimidin-4- one trifluoroacetate |
| Ex. 16 | 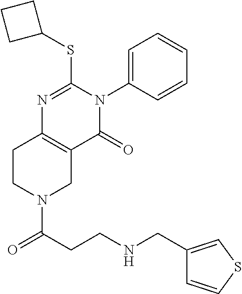 [SMILES: O=C(CCNCc1ccsc1)N1CCc2nc(SC3CCC3)n(-c3ccccc3)c(=O)c2C1] CF3COOH | 481 (M + H)+ | 1H NMR (400 MHz, DMSO-d6) δ 8.70 (s, 2H), 7.69 (ddd, J = 6.1, 3.0, 1.3 Hz, 1H), 7.64 (td, J = 4.5, 3.0 Hz, 1H), 7.55 (m, 3H), 7.31 (dd, J = 5.3, 3.8, 2.5 Hz, 2H), 7.24 (ddd, J = 6.4, 5.0, 1.3 Hz, 1H), 4.34-4.12 (m, 5H), 3.77 (t, J = 5.8 Hz, 1H), 3.70 (t, J = 5.8 Hz, 1H), 3.14 (q, J = 6.4, 5.3 Hz, 2H), 2.85 (td, J = 6.7, 4.2 Hz, 2H), 2.78-2.69 (m, 1H), 2.63 (t, J = 5.9 Hz, 1H), 2.39 (m, 2H), 2.04-1.86 (m, 4H). | 2-cyclobutyl- sulfanyl-3- phenyl-6-[3-(3- thienylmeethyl- amino)propanoyl]- 7,8-dihydro- 5H-pyrido[4, 3-d]pyrimidin- 4-one trifluoroacetate |
| Ex. 17 | 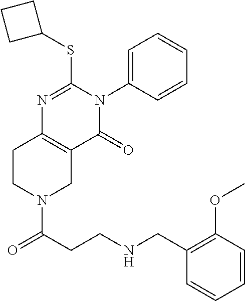 [SMILES: COc1ccccc1CNCCC(=O)N1CCc2nc(SC3CCC3)n(-c3ccccc3)c(=O)c2C1] CF3COOH | 505 (M + H)+ | 1H NMR (400 MHz, DMSO-d6) δ 8.50 (s, 2H), 7.55 (dt, ,J = 5.0, 2.0 Hz, 3H), 7.43 (m, 2H), 7.32 (m, 2H), 7.11 (dd, J = 8.2, 2.3 Hz, 1H), 7.05- 6.98 (m, 1H), 4.29 (d, J = 16.4 Hz, 2H), 4.19 (m, 3H), 3.86 (d, J = 2.4 Hz, 3H), 3.77 (t, J = 5.8 Hz, 1H), 3.70 (t, J = 5.8 Hz, 1H), 3.22-3.09 (m, 2H), 2.87 (td, J = 6.5, 4.0 Hz, 2H), 2.79- 2.69 (m, 1H), 2.64 (t, J = 5.9 Hz, 1H), 2.39 (m, 2H), 2.03-1.86 (m, 4H). | 2-cyclobutyl- sulfanyl-6-[3- [(2-methoxy- phenyl)methyl- amino]propanoyl]- 3-phenyl-7, 8-dihydro-5H- pyrido[4,3-d] pyrimidin-4- one trifluoroacetate |
| Ex. 18 | 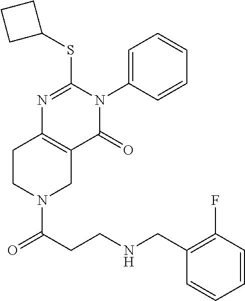 [SMILES: O=C(CCNCc1ccccc1F)N1CCc2nc(SC3CCC3)n(-c3ccccc3)c(=O)c2C1] CF3COOH | 493 (M + H)+ | 1H NMR (400 MHz, DMSO-d6) δ 8.78 (s, 2H), 7.65-7.45 (m, 5H), 7.32 (m, 4H), 4.36- 4.24 (m, 4H), 4.18 (m, 1H), 3.77 (t, J = 5.8 Hz, 1H), 3.71 (t, J = 5.8 Hz, 2H), 3.22 (d, J = 8.6 Hz, 2H), 2.88 (q, J = 6.3 Hz, 2H), 2.74 (t, J = 5.8 Hz, 1H), 2.63 (t, J = 5.9 Hz, 1H), 2.39 (m, 2H), 1.95 (m, 4H). | 2-cyclobutyl- sulfanyl-6-[3- [(2-fluoro- phenyl)methyl- ammino]propanoyl]- 3-phenyl-7,8- dihydro-5H- pyrido[4,3-d] pyrimidin-4-one trifluoroacetate |
| Ex. 19 | 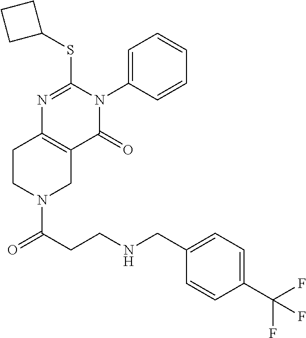 [SMILES: O=C(CCNCc1ccc(C(F)(F)F)cc1)N1CCc2nc(SC3CCC3)n(-c3ccccc3)c(=O)c2C1] CF3COOH | 543 (M + H)+ | 1H NMR (400 MHz, DMSO-d6) δ 8.84 (s, 2H), 7.86 (dd, J = 8.4, 3.4 Hz, 2H), 7.78- 7.71 (m, 2H), 7.56 (m, 3H), 7.31 (dt, J = 7.4, 2.6 Hz, 2H), 4.38-4.24 (m, 4H), 4.18 (m, 1H), 3.77 (t, J = 5.8 Hz, 1H), 3.71 (t, J = 5.8 Hz, 1H), 3.17 (d, J = 7.1 Hz, 2H), 2.87 (td, J = 7.0, 6.6, 3.2 Hz, 2H), 2.74 (t, J = 5.8 Hz, 1H), 2.63 (t, J = 6.0 Hz, 1H), 2.39 (m, 2H), 2.05-1.85 (m, 4H). | 2-cyclobutyl- sulfanyl-3- phenyl-6-[3-[[4- (trifluoromethyl) phenyl] methylamino] propanoyl]-7,8- dihydro-5H- pyrido[4,3-d] pyrimidin-4-one trifluoroacetate |
| Ex. 20 | 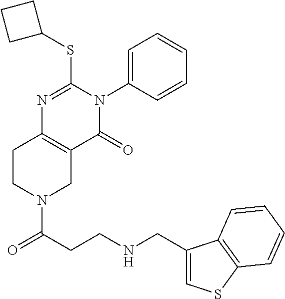 [SMILES: O=C(CCNCc1csc2ccccc12)N1CCc2nc(SC3CCC3)n(-c3ccccc3)c(=O)c2C1] CF3COOH | 531 (M + H)+ | 1H NMR (400 MHz, DMSO-d6) δ 8.89-8.72 (m, 2H), 8.06 (td, J = 7.7, 4.6 Hz, 2H), 7.99 (d, J = 7.4 Hz, 1H), 7.60-7.42 (m, 5H), 7.31 (m, 2H), 4.51 (q, J = 4.9 Hz, 2H), 4.29 (d, J = 13.3 Hz, 2H), 4.18 (m, 1H), 3.77 (t, J = 5.8 Hz, 1H), 3.70 (t, J = 5.8 Hz, 1H), 3.27 (t, J = 5.7 Hz, 3H), 2.90 (td, J = 6.7, 3.3 Hz, 2H), 2.73 (t, J = 5.6 Hz, 1H), 2.63 (t, J = 5.8 Hz, 1H), 2.38 (m, 2H), 2.05-1.85 (m, 4H). | 6-[3-(benzo- thiophen-3-yl- methylamino) propanoyl]-2- cyclobutyl- sulfanyl-3- phenyl-7,8- dihydro-5H- pyrido[4,3-d] pyrimidin-4- one trifluoroacetate |
| Ex. 21 | 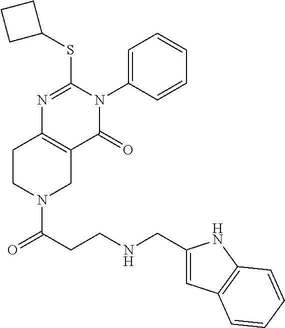 [SMILES: O=C(CCNCc1cc2ccccc2[nH]1)N1CCc2nc(SC3CCC3)n(-c3ccccc3)c(=O)c2C1] CF3COOH | 514 (M + H)+ | 1H NMR (400 MHz, DMSO-d6) δ 11.20-11.09 (m, 1H), 8.80 (s, 2H), 7.55 (dt, J = 5.2, 2.9 Hz, 4H), 7.47-7.41 (m, 1H), 7.31 (dt, J = 7.4, 2.5 Hz, 2H), 7.14 (m, 1H), 7.07- 6.99 (m, 1H), 6.65-6.58 (m, 1H), 4.41-4.23 (m, 4H), 4.18 (m, 1H), 3.77 (t, J = 5.8 Hz, 1H), 3.69 (t, J = 5.8 Hz, 1H), 3.21 (q, J = 6.0, 5.5 Hz, 2H), 2.88 (td, J = 6.6, 2.3 Hz, 2H), 2.72 (t, J = 6.0 Hz, 1H), 2.63 (t, J = 6.0 Hz, 1H), 2.39 (m, 2H), 2.05-1.86 (m, 4H). | 2-cyclobutyl- sulfanyl-6-[3- (1H-indol-2- ylmethylamino) propanoyl]-3- phenyl-7,8- dihydro-5H- pyrido[4,3-d] pyrimidin-4-one trifluoroacetate |
| Ex. 22 | 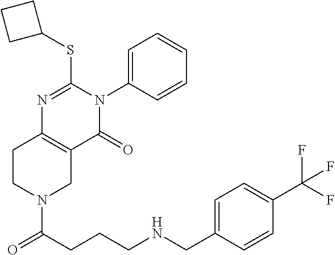 [SMILES: O=C(CCCNCc1ccc(C(F)(F)F)cc1)N1CCc2nc(SC3CCC3)n(-c3ccccc3)c(=O)c2C1] CF3COOH | 557 (M + H)+ | 1H NMR (400 MHz, DMSO-d6) δ 8.86 (s, 2H), 7.85 (dd, J = 8.4, 3.6 Hz, 2H), 7.73 (dd, J = 8.3, 4.0 Hz, 2H), 7.55 (m, 3H), 7.36-7.27 (m, 2H), 4.28 (d, J = 4.2 Hz, 4H), 4.18 (m, 1H), 3.75 (t, J = 5.8 Hz, 1H), 3.70 (t, J = 5.8 Hz, 1H), 3.01 (d, J = 5.9 Hz, 2H), 2.73 (t, J = 5.7 Hz, 1H), 2.59 (m, 4H), 2.39 (m, 2H), 2.02-1.82 (m, 6H). | 2-cyclobutyl- sulfanyl-3- phenyl-6-[4-[[4- trifluoromethyl) phenyl] methylamino] butanoyl]-7,8- dihydro-5H- pyrido[4,3-d] pyrimidin-4-one trifluoroacetate |
| Ex. 23 | 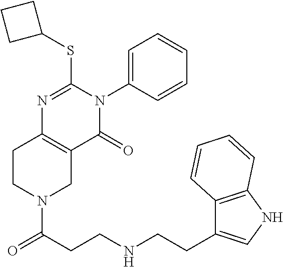 [SMILES: O=C(CCNCCc1c[nH]c2ccccc12)N1CCc2nc(SC3CCC3)n(-c3ccccc3)c(=O)c2C1] CF3COOH | 528 (M + H)+ | 1H NMR (400 MHz, DMSO-d6) δ 10.96 (d, J = 4.5 Hz, 1H), 8.35 (br, 2H), 7.69-7.42 (m, 4H), 7.37 (d, J = 8.0 Hz, 1H), 7.34-7.28 (m, 2H), 7.25 (dd, 4.7, 2.3 Hz, 1H), 7.10 (dd, J = 7.5, 7.4 Hz, 1H), 7.02 (dd, J = 7.5, 7.4 Hz, 1H), 4.30 (d, J = 11.7 Hz, 2H), 4.24-4.11 (m, 1H), 3.78 (t, J = 5.8 Hz, 1H), 3.71 (t, J = 5.9 Hz, 1H), 3.46-3.31 (m, 1H), 3.31-3.17 (m, 3H), 3.05 (td, J = 7.2, 7.1 Hz, 2H), 2.93-2.79 (m, 2H), 2.79-2.71 (m, 1H), 2.71- 2.60 (m, 1H), 2.44-2.31 (m, 2H), 2.06-1.87 (m, 4H). | 2-cyclobutyl- sulfanyl-6-[3- [2-(1H-indol- 3-yl)ethylamino] propanoyl]- 3-phenyl-7,8- dihydro-5H- pyrido[4,3-d] pyrimidin-4-one trifluoroacetate) |
| Ex. 24 | 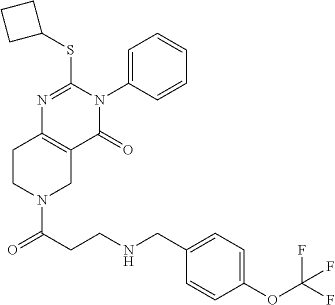 [SMILES: O=C(CCNCc1ccc(OC(F)(F)F)cc1)N1CCc2nc(SC3CCC3)n(-c3ccccc3)c(=O)c2C1] CF3COOH | 559 (M + H)+ | 1H NMR (400 MHz, DMSO-d6) δ 8.76 (s, 2H), 7.68-7.62 (m, 2H), 7.55 (dt, J = 5.0, 2.2 Hz, 3H), 7.51-7.41 (m, 2H), 7.34-7.28 (m, 2H), 4.35- 4.12 (m, 5H), 3.77 (t, J = 5.8 Hz, 1H), 3.70 (t, J = 5.8 Hz, 1H), 3.17 (s, 2H), 2.87 (td, J = 6.6, 3.9 Hz, 2H), 2.74 (t, J = 5.8 Hz, 1H), 2.39 (m, 2H), 1.95 (m, 4H). | 2-cyclobutyl- sulfanyl-3- phenyl-6-[3-[[4- (trifluoro- methoxy)phenyl] methylamino] propanoyl]-7,8- dihydro-5H- pyrido[4,3-d] pyrimidin-4-one trifluoroacetate) |

### Example 28

### Synthesis of 6-[3-(1,3-Benzodioxol-5-ylmethylamino)propanoyl]-2-cyclobut ylsulfanyl-3-phenyl-7,8-dihydro-5H-pyrido[4,3-d]pyrimidin-4-one Trifluoroacetate (Compound of the Following Formula (XX))

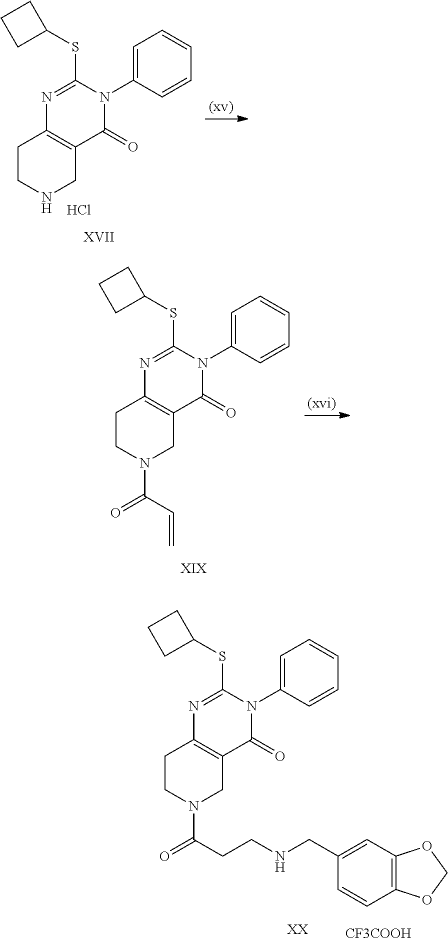

(Step (xv))

To a dichloromethane (15 ml) solution of Compound XVIII (which was the same as Compound VI of Reference Example 1 described above) (2.0 g), acryloyl chloride (693 mg) and DIPEA (2.99 ml) were added, followed by stirring at room temperature for 1 hour. Dichloromethane and 1 N HCl were added, followed by extraction with dichloromethane, and drying over anhydrous sodium sulfate. Purification was carried out by using a preparative silica gel column (ethyl acetate:hexane=1:4) to obtain 1.7 g of Compound XIX.

(Step (xvi))

Compound XIX (48 mg) was dissolved in tetrahydrofuran (0.5 ml). Diazabicycloundecene (58 μl) and piperonylamine (24 μl) were added, followed by stirring at 80° C. for 15 hours. After concentration under reduced pressure, purification was carried out by using a reversed-phase preparative column to obtain Compound XX (61 mg). (Yield: 73%)

Note that the compound (Compound XVII) of Example 10 was also successfully synthesized from Compound XIX described above on the basis of the same production method as described above. Specifically, Compound XIX (50 mg) was dissolved in 1,4-dioxane (1.0 ml). Diazabicycloundecene (61 μl) and benzylamine (30 μl) were added, followed by stirring at 80° C. for 15 hours. After concentration under reduced pressure, purification was carried out by using a reversed-phase preparative column to successfully obtain Compound XVII (54 mg). (Yield: 67%)

### Examples 25 to 27 and 29 to 49, Example 56, Example 59, and Example 60

Compounds of Examples 25 to 27 and 29 to 49, Example 56, Example 59, and Example 60 shown in the table below were synthesized according to the method of Example 28.

### Examples 50 to 55, Example 57, and Example 58

Synthesis in Examples 50 to 55, Example 57, and Example 58 was carried out according to the method in Example 1.

Chloroacetyl chloride was used instead of bromoacetyl bromide used in Example 1. By a subsequent reaction with a suitable amine, the compounds of Examples 50 to 55, Example 57, and Example 58 shown in the table below were synthesized.

TABLE 2

| | | MASS | | |
|---|---|---|---|---|
| | | (ESI) | | Compound |
| Compound | | m/z | | Name |
| No. | | (M + H)+ | NMR | (IUPAC) |
| Ex. 25 | 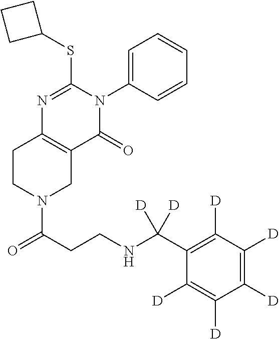 [SMILES: [2H]c1c([2H])c([2H])c(C([2H])([2H])NCCC(=O)N2CCc3nc(SC4CCC4)n(-c4ccccc4)c(=O)c3C2)c([2H])c1[2H]] CF3COOH | 482 (M + H)+ | ¹H NMR (400 MHz, DMSO-d₆) δ 8.83-8.67 (br, 2H), 7.55 (dt, J = 4.7, 2.3 Hz, 3H), 7.31 (dt, J = 7.3, 2.5 Hz, 2H), 4.29 (d, J = 13.2 Hz, 1H), 4.24-4.13 (m, 1H), 3.77 (t, J = 5.8 Hz, 1H), 3.70 (t, J = 5.7 Hz, 1H), 3.22-3.09 (m, 2H), 2.93-2.82 (m, 2H), 2.74 (t, J = 5.8 Hz, 1H), 2.63 (t, J = 6.2 Hz, 1H), 2.45-2.33 (m, 2H), 2.03- 1.86 (m, 4H). | 2-cyclobutyl- sulfanyl-6-[3- [[dideuterio- (2,3,4,5,6- pentadeuterio- phenyl)methyl] amino]propanoyl]- 3-phenyl-7,8- dihydro-5H- pyrido[4,3-d] pyrimidin-4-one trifluoroacetate |
| Ex. 26 | 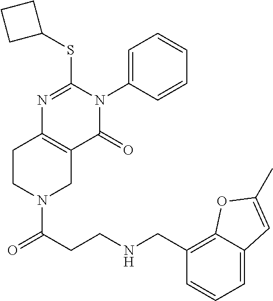 [SMILES: Cc1cc2cccc(CNCCC(=O)N3CCc4nc(SC5CCC5)n(-c5ccccc5)c(=O)c4C3)c2o1] CF3COOH | 529 (M + H)+ | ¹H NMR (400 MHz, DMSO-d₆) δ 8.84 (s, 2H), 7.63-7.51 (m, 4H), 7.37 (ddd, J = 7.0, 5.5, 1.3 Hz, 1H), 7.34- 7.24 (m, 3H), 6.68 (dd, J = 2.4, 1.2 Hz, 1H), 4.47 (q, J = 4.7 Hz, 2H), 4.29 (d, J = 14.0 Hz, 2H), 4.24-4.12 (m, 1H), 3.77 (t, J = 5.8 Hz, 1H), 3.71 (t, J = 5.9 Hz, 1H), 3.24 (d, J = 9.2 Hz, 3H), 2.95-2.84 (m, 2H), 2.74 (t, J = 5.7 Hz, 1H), 2.63 (t, J = 5.7 Hz, 1H), 2.44-2.30 (m, 3H), 2.04-1.84 (m, 4H). | 2-cyclobutyl- sulfanyl-6- [3-[(2-methyl- benzofuran- 7-yl)methylamino] propanoyl]-3- phenyl-7,8- dihydro-5H- pyrido[4,3-d] pyrimidin-4-one trifluoroacetate |
| Ex. 27 | 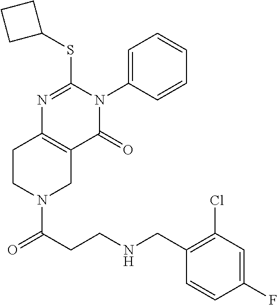 [SMILES: O=C(CCNCc1ccc(F)cc1Cl)N1CCc2nc(SC3CCC3)n(-c3ccccc3)c(=O)c2C1] CF3COOH | 527 (M + H)+ | ¹H NMR (400 MHz, DMSO-d₆) δ 8.77 (s, 2H), 7.71 (dt, J = 8.7, 5.9 Hz, 1H), 7.62 (dt, J = 8.8, 2.7 Hz, 1H), 7.59- 7.50 (m, 3H), 7.39 (tt, J = 8.5, 2.8 Hz, 1H), 7.28- 7.35 (m, 2H), 4.38-4.24 (qd, 4H), 4.18 (qd, J = 8.5, 7.9, 4.4 Hz, 1H), 3.78 (t, J = 5.8 Hz, 1H), 3.71 (t, J = 5.8 Hz, 1H), 3.24 (s, 2H), 2.89 (q, J = 6.2 Hz, 2H), 2.74 (t, J = 5.9 Hz, 1H), 2.64 (t, J = 5.8 Hz, 1H), 2.45-2.30 (m, 2H), 2.06- 1.84 (m, 4H). | 6-[3-[(3- chloro-4- fluoro-phenyl) methylamino] propanoyl]- 2-cyclobutyl- sulfanyl-3- phenyl-7,8- dihydro-5H- pyrido[4,3- d]pyrimidin- 4-one trifluoroacetate |
| Ex. 28 | 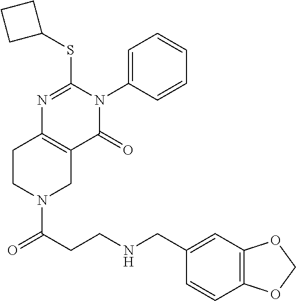 [SMILES: O=C(CCNCc1ccc2c(c1)OCO2)N1CCc2nc(SC3CCC3)n(-c3ccccc3)c(=O)c2C1] CF3COOH | 519 (M + H)+ | ¹H NMR (400 MHz, DMSO-d₆) δ 8.83-8.67 (br, 2H), 7.55 (dt, J = 4.7, 2.3 Hz, 3H), 7.31 (dt, J = 7.3, 2.5 Hz, 2H), 4.29 (d, J = 13.2 Hz, 2H), 4.24-4.13 (m, 1H), 3.77 (t, J = 5.8 Hz, 1H), 3.70 (t, J = 5.7 Hz, 1H), 3.22-3.09 (m, 2H), 2.93-2.82 (m, 2H), 2.74 (t, J = 5.8 Hz, 1H), 2.63 (t, J = 6.2 Hz, 1H), 2.45-2.33 (m, 2H), 2.03- 1.86 (m, 4H). | 6-[3-(1,3- benzodioxol- 5-ylmethyl- amino)propanoyl]- 2-cyclo- butylsulfanyl- 3-phenyl-7,8- dihydro-5H- pyrido[4,3-d] pyrimidin-4-one trifluoroacetate |
| Ex. 29 | 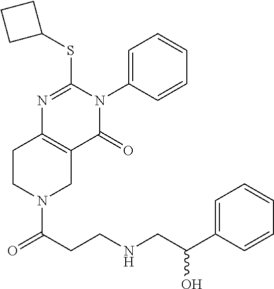 [SMILES: O=C(CCNCC(O)c1ccccc1)N1CCc2nc(SC3CCC3)n(-c3ccccc3)c(=O)c2C1] CF3COOH | 505 (M + H)+ | ¹H NMR (400 MHz, DMSO-d₆) δ 8.75-8.67 (br, 1H), 8.51- 8.43 (br, 1H), 7.60- 7.51 (m, 3H), 7.45-7.35 (m, 4H), 7.38-7.27 (m, 3H), 6.20 (br, 1H), 4.97-4.87 (m, 1H), 4.30 (d, J = 10.0 Hz, 2H), 4.24-4.14 (m, 1H), 3.78 (t, J = 5.9 Hz, 1H), 3.72 (t, J = 5.8 Hz, 1H), 3.30-3.17 (m, 3H), 3.12-3.01 (m, 1H), 2.92 (q, J = 6.9 Hz, 2H), 2.75 (t, J = 5.8 Hz, 1H), 2.64 (t, J = 5.9 Hz, 1H), 2.45-2.34 (m, 2H), 2.02- 1.88 (m, 4H). | 2-cyclobutyl- sulfanyl-6- [3-[(2- hydroxy-2- phenyl-ethyl) amino]propanoyl]- 3-phenyl- 7,8-dihydro- 5H-pyrido[4,3-d] pyrimidin-4-one trifluoroacetate |
| Ex. 30 | 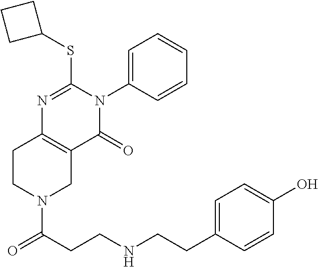 [SMILES: O=C(CCNCCc1ccc(O)cc1)N1CCc2nc(SC3CCC3)n(-c3ccccc3)c(=O)c2C1] CF3COOH | 505 (M + H)+ | ¹H NMR (400 MHz, DMSO-d₆) δ 9.44-9.20 (br, 1H), 8.52- 8.31 (br, 2H), 7.62- 7.51 (m, 3H), 7.37-7.27 (m, 2H), 7.10-7.00 (m, 2H), 6.77-6.67 (m, 2H), 4.30 (d, J = 12.2 Hz, 2H), 4.25-4.12 (m, 1H), 3.78 (t, J = 5.8 Hz, 1H), 3.72 (t, J = 5.8 Hz, 1H), 3.26-3.04 (m, 4H), 2.94- 2.70 (m, 5H), 2.69-2.59 (m, 1H), 2.45-2.31 (m, 2H), 2.04-1.85 (m, 4H). | 2-cyclobutyl- sulfanyl-6- [3-[2-(4- hydroxyphenyl) ethylamino] propanoyl]-3- phenyl- 7,8-dihydro- 5H-pyrido[4,3- d]pyrimidin- 4-one trifluoroacetate |
| Ex. 31 | 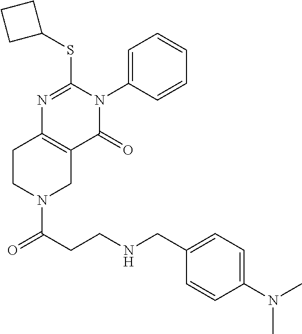 [SMILES: CN(C)c1ccc(CNCCC(=O)N2CCc3nc(SC4CCC4)n(-c4ccccc4)c(=O)c3C2)cc1] CF3COOH | 518 (M + H)+ | ¹H NMR (400 MHz, DMSO-d₆) δ 9.44-9.20 (br, 1H), 8.52- 8.31 (br, 2H), 7.62- 7.51 (m, 3H), 7.37-7.27 (m, 2H), 7.10-7.00 (m, 2H), 6.77-6.67 (m, 2H), 4.30 (d, J = 12.2 Hz, 2H), 4.25-4.12 (m, 1H), 3.78 (t, J = 5.8 Hz, 1H), 3.72 (t, J = 5.8 Hz, 1H), 3.26-3.04 (m, 4H), 2.94- 2.70 (m, 5H), 2.69-2.59 (m, 1H), 2.45-2.31 (m, 2H), 2.04-1.85 (m, 4H). | 2-cyclobutyl- sulfanyl-6- [3-[(4- dimethylamino- phenyl) methylamino] propanoyl]-3- phenyl-7,8- dihydro-5H- pyrido[4,3-d] pyrimidin-4- one trifluoroacetate |
| Ex. 32 | 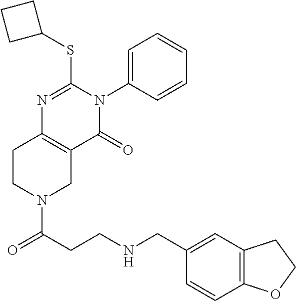 [SMILES: O=C(CCNCc1ccc2c(c1)CCO2)N1CCc2nc(SC3CCC3)n(-c3ccccc3)c(=O)c2C1] CF3COOH | 517 (M + H)+ | ¹H NMR (400 MHz, DMSO-d₆) δ 8.56 (s, 2H), 7.61-7.50 (m, 3H), 7.39-7.27 (m, 3H), 7.22 (ddd, J = 7.8, 5.5, 1.9 Hz, 1H), 6.82 (dd, J = 8.2, 4.1 Hz, 1H), 4.62-4.50 (m, 2H), 4.28 (d, J = 14.4 Hz, 2H), 4.23-4.05 (m, 3H), 3.77 (t, J = 5.8 Hz, 1H), 3.70 (t, J = 5.9 Hz, 1H), 3.19 (td, J = 8.8, 3.0 Hz, 2H), 3.16- 3.05 (m, 2H), 2.84 (q, J = 6.2 Hz, 2H), 2.70-2.59 (m, 2H), 2.44-2.30 (m, 2H), 2.03-1.86 (m, 4H). | 2-cyclobutyl- sulfanyl-6- [3-(2,6- dihydrobenzo- furan-5-yl- methylamino) propanoyl]-3- phenyl-7,8- dihydro-5H- pyrido[4,3- d]pyrimidin- 4-one trifluoroacetate |
| Ex. 33 | 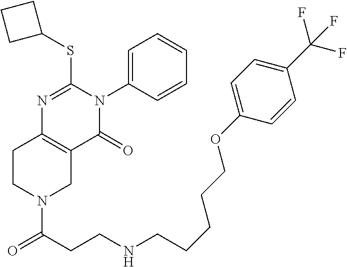 [SMILES: O=C(CCNCCCCCOc1ccc(C(F)(F)F)cc1)N1CCc2nc(SC3CCC3)n(-c3ccccc3)c(=O)c2C1] CF3COOH | 615 (M + H)+ | ¹H NMR (400 MHz, DMSO-d₆) δ 8.25 (s, 2H), 7.68-7.62 (m, 2H), 7.59-7.52 (m, 3H), 7.35-7.28 (m, 2H), 7.14- 7.08 (m, 2H), 4.30 (d, J = 13.5 Hz, 2H), 4.06 (td, J = 6.3, 3.1 Hz, 2H), 3.78 (t, J = 5.8 Hz, 1H), 3.72 (t, J = 5.8 Hz, 1H), 3.17 (q, J = 6.2 Hz, 2H), 2.97 (s, 2H), 2.85 (q, J = 6.6 Hz, 2H), 2.75 (t, J = 5.7 Hz, 1H), 2.44-2.31 (m, 2H), 2.04- 1.87 (m, 4H), 1.76 (ddt, J = 10.3, 6.2, 3.7 Hz, 2H), 1.71- 1.59 (m, 2H), 1.53-1.40 (m, 2H). | 2-cyclobutyl- sulfanyl-3- phenyl-6-[3- [5-[4-(tri- fluoromethyl) phenoxy] pentylamino] propanoyl]- 7,8-dihydro- 5H-pyrido [4,3-d] pyrimidin-4-one trifluoroacetate |
| Ex. 34 | 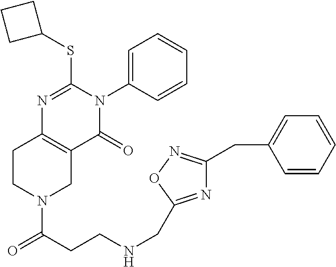 [SMILES: O=C(CCNCc1nc(Cc2ccccc2)no1)N1CCc2nc(SC3CCC3)n(-c3ccccc3)c(=O)c2C1] CF3COOH | 557 (M + H)+ | ¹H NMR (400 MHz, DMSO-d₆) δ 9.56-9.03 (br, 2H), 7.54- 7.44 (m, 3H), 7.32-7.16 (m, 7H), 4.61 (s, 2H), 4.22 (d, J = 14.3 Hz, 2H), 4.16-4.06 (m, 3H), 3.70 (t, J = 5.8 Hz, 1H), 3.63 (t, J = 5.9 Hz, 1H), 3.31-3.21 (m, 2H), 2.89- 2.79 (m, 2H), 2.67 (t, J = 5.7 Hz, 1H), 2.56 (t, J = 5.9 Hz, 1H), 2.38-2.27 (m, 2H), 1.95-1.79 (m, 4H). | 6-[3-[(3- benzyl-1,2,4- oxadiazol-5- yl)methyl- amino]propanoyl]- 2-cyclobutyl- sulfanyl-3-phenyl- 7,8-dihydro-5H- pyrido[4,3-d] pyrimidin-4-one trifluoroacetate |
| Ex. 35 | 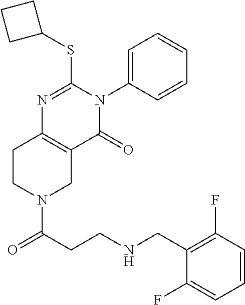 [SMILES: O=C(CCNCc1c(F)cccc1F)N1CCc2nc(SC3CCC3)n(-c3ccccc3)c(=O)c2C1] CF3COOH | 511 (M + H)+ | ¹H NMR (400 MHz, DMSO-d₆) δ 8.92-8.75 (br, 2H), 7.65- 7.50 (m, 4H), 7.35-7.29 (m, 2H), 7.29-7.20 (m, 2H), 4.33-4.24 (m, 4H), 4.24- 4.11 (m, 1H), 3.77 (t, J = 5.8 Hz, 1H), 3.71 (t, J = 5.8 Hz, 1H), 3.31-3.18 (m, 2H), 2.93-2.83 (m, 2H), 2.74 (t, J = 5.8 Hz, 1H), 2.64 (t, J = 5.7 Hz, 1H), 2.44-2.33 (m, 2H), 2.04- 1.85 (m, 4H). | 2-cyclobutyl- sulfanyl-6- [3-[(2,6- difluorophenyl) methylamino] propanoyl]- 3-phenyl-7,8- dihydro-5H- pyrido[4,3-d] pyrimidin-4-one trifluoroacetate |
| Ex. 36 | 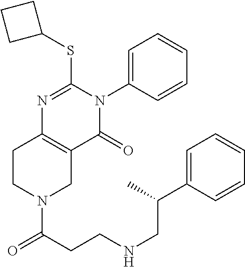 [SMILES: C[C@@H](CNCCC(=O)N1CCc2nc(SC3CCC3)n(-c3ccccc3)c(=O)c2C1)c1ccccc1] CF3COOH | 503 (M + H)+ | ¹H NMR (400 MHz, DMSO-d₆) δ 8.58-8.36 (br, 1H), 8.30- 8.11 (br, 1H), 7.61- 7.51 (m, 3H), 7.42-7.22 (m, 7H), 4.27 (d, J = 7.7 Hz, 2H), 4.19 (ttd, J = 8.7, 8.0, 3.5 Hz, 1H), 3.75 (t, J = 5.9 Hz, 1H), 3.70 (t, J = 5.8 Hz, 1H), 3.27-3.07 (m, 5H), 2.91- 2.80 (m, 2H), 2.78-2.70 (m, 1H), 2.66-2.59 (m, 1H), 2.45-2.33 (m, 2H), 2.02- 1.86 (m, 4H), 1.31-1.22 (m, 3H). | 3-cyclobutyl- sulfanyl- 3-phenyl-6- [3-[[(2R)-2- phenylpropyl] amino]propanoyl]- 7,8-dihydro-5H- pyrido[4,3-d] pyrimidin-4-one trifluoroacetate |
| Ex. 37 | 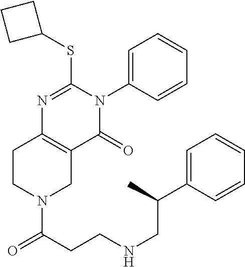 [SMILES: C[C@H](CNCCC(=O)N1CCc2nc(SC3CCC3)n(-c3ccccc3)c(=O)c2C1)c1ccccc1] CF3COOH | 503 (M + H)+ | ¹H NMR (400 MHz, DMSO-d₆) δ 8.60-8.39 (br, 1H), 8.32- 8.13 (br, 1H), 7.60- 7.50 (m, 3H), 7.41-7.22 (m, 7H), 4.27 (d, J = 7.6 Hz, 2H), 4.24-4.11 (m, 1H), 3.75 (t, J = 5.8 Hz, 1H), 3.70 (t, J = 5.8 Hz, 1H), 3.30-3.06 (m, 5H), 2.92-2.81 (m, 2H), 2.74 (t, J = 5.8 Hz, 1H), 2.63 (t, J = 5.8 Hz, 1H), 2.44-2.31 ( m, 2H), 2.02- 1.88 (m, 4H), 1.34-1.20 (m, 3H). | 2-cyclobutyl- sulfanyl-3- phenyl-6- [3-[[(2S)-2- phenylpropyl] amino]propanoyl]- 7,8-dihydro-5H- pyrido[4,3-d] pyrimidin- 4-one trifluoroacetate |
| Ex. 38 | 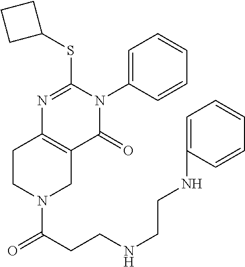 [SMILES: O=C(CCNCCNc1ccccc1)N1CCc2nc(SC3CCC3)n(-c3ccccc3)c(=O)c2C1] CF3COOH | 504 (M + H)+ | ¹H NMR (400 MHz, DMSO-d₆) δ 8.28 (s, 2H), 7.56-7.43 (m, 3H), 7.29-7.20 (m, 2H), 7.10-7.00 (m, 2H), 6.61- 6.49 (m, 3H), 4.23 (d, J = 14.2 Hz, 2H), 4.18-4.06 (m, 1H), 3.71 (t, J = 5.8 Hz, 1H), 3.16 (d, J = 7.1 Hz, 2H), 3.07 (s, 2H), 2.80 (q, J = 6.4 Hz, 2H), 2.67 (t, J = 5.8 Hz, 1H), 2.57 (t, J = 5.9 Hz, 1H), 2.38-2.24 (m, 2H), 1.98-1.78 (m, 4H). | 6-[3-(2- aminolinoethyl- amino)propanoyl]- 2-cyclobutyl- sulfanyl-3-phenyl- 7,8-dihydro- 5H-pyrido [4,3-d]pyrimidin- 4-one trifluoroacetate |
| Ex. 39 | 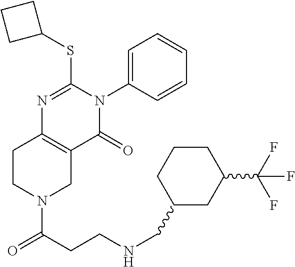 [SMILES: O=C(CCNCC1CCCC(C(F)(F)F)C1)N1CCc2nc(SC3CCC3)n(-c3ccccc3)c(=O)c2C1] CF3COOH | 549 (M + H)+ | ¹H NMR (400 MHz, DMSO-d₆) δ 8.47-8.26 (br, 2H), 7.61- 7.50 (m, 3H), 7.36-7.26 (m, 2H), 4.30 (d, J = 11.8 Hz, 2H), 4.26-4.12 (m, 1H), 3.78 (t, J = 5.9 Hz, 1H), 3.72 (t, J = 5.7 Hz, 1H), 3.22-3.10 (m, 2H), 3.08-2.98 (m, 1H), 2.95-2.81 (m, 4H), 2.79- 2.71 (m, 1H), 2.68-2.60 (m, 1H), 2.45-2.23 (m, 3H), 2.19-2.14 (m, 1H), 2.05- 1.88 (m, 4H), 1.88-1.68 (m, 3H), 1.65-1.41 (m, 1H), 1.39-1.24 (m, 1H), 1.22- 1.08 (m, 1H), 1.06-0.84 (m, 1H). | 2-cyclobutyl- sulfanyl-3- phenyl-6- [3-[[3-(tri- fluoromethyl) cyclohexyl] methylamino] propanoyl]- 7,8-dihydro- 5H-pyrido [4,3-d] pyrimidin-4- one trifluoroacetate |
| Ex. 40 | 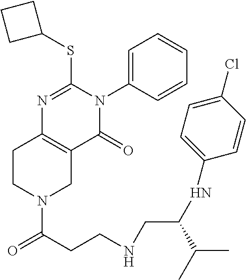 [SMILES: CC(C)[C@H](CNCCC(=O)N1CCc2nc(SC3CCC3)n(-c3ccccc3)c(=O)c2C1)Nc1ccc(Cl)cc1] CF3COOH | 581 (M + H)+ | ¹H NMR (400 MHz, DMSO-d₆) δ 8.37 (brs, 1H), 8.14 (brs, 1H), 7.62-7.48 (m, 3H), 7.39-7.26 (m, 2H), 7.16-7.07 (m, 2H), 6.76- 6.61 (m, 2H), 5.71 (dd, J = 4.5, 10.0 Hz, 1H), 4.28 (d, J = 9.9 Hz, 2H), 4.23-4.09 (m, 1H), 3.76 (t, J = 5.9 Hz, 1H), 3.70 (t, J = 5.8 Hz, 1H), 3.64-3.55 (m, 1H), 3.29- 3.12 (m, 3H), 2.98-2.80 (m, 3H), 2.80-2.69 (m, 1H), 2.67-2.58 (m, 1H), 2.45- 2.30 (m, 2H), 2.05-1.78 (m, 5H), 0.94-0.82 (m, 6H). | 6-[3-[[(2R)- 2-(4-chloro- anilino)-3- methyl- butyl]amino] propanoyl]-2- cyclobutyl- sulfanyl-3- phenyl-7,8- dihydro-5H- pyrido[4,3-d] pyrimidin-4-one trifluoroacetate |
| Ex. 41 | 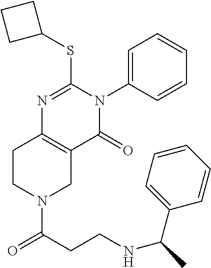 [SMILES: C[C@@H](NCCC(=O)N1CCc2nc(SC3CCC3)n(-c3ccccc3)c(=O)c2C1)c1ccccc1] CF3COOH | 490 (M + H)+ | ¹H NMR (400 MHz, DMSO-d₆) δ 8.96 (brs, 1H), 8.70 (brs, 1H), 7.62-7.51 (m, 5H), 7.51-7.38 (m, 3H), 7.37-7.26 (m, 2H), 4.55- 4.36 (m, 1H), 4.26 (d, J = 19.3 Hz, 2H), 4.22-4.11 (m, 1H), 3.77-3.72 (m, 1H), 3.67 (t, J = 5.8 Hz, 1H), 3.15-2.99 (m, 1H), 2.99- 2.86 (m, 1H), 2.86-2.58 (m, 2H), 2.77-2.69 (m, 1H), 2.65-2.58 (m, 1H), 2.45- 2.28 (m, 2H), 2.05-1.85 (m, 4H), 1.57 (t, J = 6.2 Hz, 3H). | 2-cyclobutyl- sulfanyl-3- phenyl-6- [3-[[(1R)-1- phenylethyl] amino]propanoyl]- 7,8-dihydro-5H- pyrido[4,3- d]pyrimidin- 4-one trifluoroacetate |
| Ex. 42 | 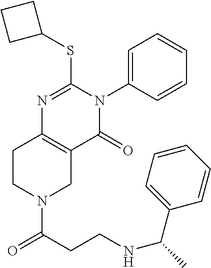 CF3COOH | 450 (M + H)+ | ¹H NMR (400 MHz, DMSO-d₆) δ 8.92 (brs, 1H), 8.68 (brs, 1H), 7.64-7.41 (m, 8H), 7.37-7.25 (m, 2H), 4.51-4.37 (m, 1H), 4.26 (d, J = 19.6 Hz, 2H), 4.22-4.12 (m, 1H), 3.75 (t, J = 6.1 Hz, 1H), 3.68 (t, J = 5.8 Hz, 1H), 3.17-2.99 (m, 1H), 2.98- 2.86 (m, 1H), 2.86-2.76 (m, 2H), 2.76-2.58 (m, 1H), 2.67-2.58 (m, 1H), 2.46- 2.28 (m, 2H), 2.06-1.84 (m, 4H), 1.57 (dd, J = 6.8, 5.6 Hz, 3H). | 2-cyclobutyl- sulfanyl-3- phenyl-6- [3-[[(1S)-1- phenylethyl] amino]propanoyl]- 7,8-dihydro-5H- pyrido[4,3- d]pyrimidin- 4-one trifluoroacetate |
| Ex. 43 | 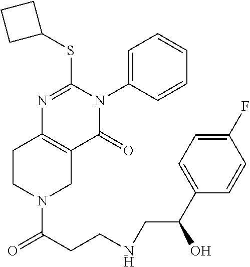 [SMILES: O=C(CCNC[C@H](O)c1ccc(F)cc1)N1CCc2nc(SC3CCC3)n(-c3ccccc3)c(=O)c2C1] CF3COOH | 524 (M + H)+ | ¹H NMR (400 MHz, DMSO-d₆) δ 8.87 (brs, 1H), 8.73- 8.33 (m, 1H), 7.72-7.40 (m, 5H), 7.40-7.20 (m, 4H), 6.26 (brs, 1H), 5.04- 4.79 (m, 1H), 4.40-4.10 (m, 3H), 3.92-3.55 (m, 3H), 3.34-3.14 (m, 2H), 3.14- 2.99 (m, 1H), 2.99-2.80 (m, 2H), 2.80-2.69 (m, 1H), 2.68-2.59 (m, 1H), 2.48- 2.28 (m, 2H), 2.10-1.78 (m, 4H). | 2-cyclobutyl- sulfanyl-6- [3-[[(2R)- 2-(4-fluoro- phenyl)-2- hydroxy-ethyl] amino]propanoyl]- 3-phenyl-7,8- dihydro-5H- pyrido[4,3- d]pyrimidin- 4-one trifluoroacetate |
| Ex. 44 | 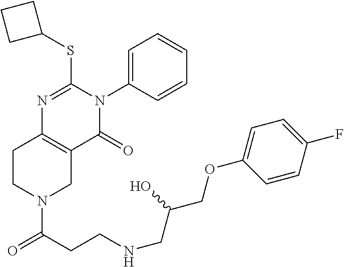 [SMILES: O=C(CCNCC(O)COc1ccc(F)cc1)N1CCc2nc(SC3CCC3)n(-c3ccccc3)c(=O)c2C1] CF3COOH | 554 (M + H)+ | ¹H NMR (400 MHz, DMSO-d₆) δ 8.62-8.30 (m, 2H), 7.68- 7.50 (m, 3H), 7.37-7.27 (m, 2H), 7.22-7.08 (m, 2H), 7.05-6.94 (m, 2H), 5.90 (s, 1H), 4.30 (d, J = 12.6 Hz, 2H), 4.25-4.09 (m, 2H), 4.01- 3.87 (m, 2H), 3.85-3.65 (m, 2H), 3.31-3.16 (m, 2H), 3.14-2.98 (m, 1H), 2.98- 2.82 (m, 2H), 2.82-2.71 (m, 1H), 2.70-2.61 (m, 1H), 2.70 (dt, J = 45.82, 5.87 Hz, 2H), 2.48-2.29 (m, 2H), 2.09-1.82 (m, 4H). | 2-cyclobutyl- sulfanyl-6- [3-[[3-(4- fluorophenoxy)- 2-hydroxy- propyl]amino] propanoyl]-7,8- dihydro-5H- pyrido[4,3-d] pyrimidin-4- one trifluoroacetate |
| Ex. 45 | 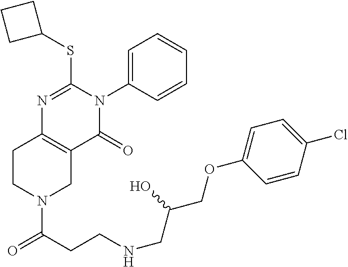 [SMILES: O=C(CCNCC(O)COc1ccc(Cl)cc1)N1CCc2nc(SC3CCC3)n(-c3ccccc3)c(=O)c2C1] CF3COOH | 570 (M + H)+ | ¹H NMR (400 MHz, DMSO-d₆) δ 8.47 (brs, 2H), 7.69- 7.47 (m, 3H), 7.47-7.24 (m, 4H), 7.00 (d, J = 8.95 Hz, 2H), 6.10-5.81 (m, 1H), 4.30 (d, J = 12.8 Hz, 2H), 4.24-4.09 (m, 2H), 4.08-3.87 (m, 3H), 3.87-3.63 (dt, J = 24.1, 5.88 Hz, 2H), 3.30-3.15 (m, 2H), 3.14-2.99 (m, 1H), 2.99-2.82 (m, 2H), 2.82- 2.70 (m, 1H), 2.71-2.58 (m, 1H), 2.46-2.29 (m, 2H), 2.09-1.80 (m, 4H). | 6-[3-[[3-(4- chlorophenoxy)- 2-hydroxy-propyl] amino]propanoyl]- 2-cyclobutyl- sulfanyl- 3-phenyl-7,8- dihydro-5H-pyrido [4,3-d] pyrimidin-4- one trifluoroacetate |
| Ex. 46 | 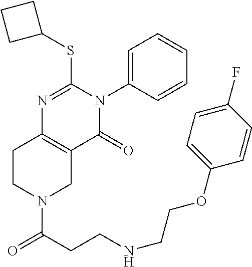 [SMILES: O=C(CCNCCOc1ccc(F)cc1)N1CCc2nc(SC3CCC3)n(-c3ccccc3)c(=O)c2C1] CF3COOH | 524 (M + H)+ | ¹H NMR (400 MHz, DMSO-d₆) δ 8.57 (brs, 2H), 7.61- 7.51 (m, 3H), 7.36-7.28 (m, 2H), 7.21-7.12 (m, 2H), 7.05-6.98 (m, 2H), 4.30 (d, J = 12.7 Hz, 2H), 4.26-4.13 (m, 3H), 3.78 (t, J = 5.9 Hz, 1H), 3.72 (t, J = 5.8 Hz, 1H), 3.47-3.28 (m, 2H), 3.32- 3.22 (m, 2H), 2.98-2.82 (m, 2H), 2.81-2.70 (m, 1H), 2.70-2.59 (m, 1H), 2.45- 2.34 (m, 2H), 2.05-1.86 (m, 4H). | 2-cyclobutyl- sulfanyl-6- [3-[2-(4- fluorophenoxy) ethylamino] propanoyl]- 3-phenyl- 7,8-dihydro- 5H-pyrido [4,3-d]pyrimidin- 4-one trifluoroacetate |
| Ex. 47 | 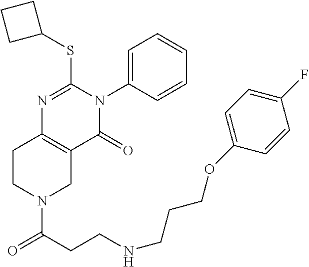 [SMILES: O=C(CCNCCCOc1ccc(F)cc1)N1CCc2nc(SC3CCC3)n(-c3ccccc3)c(=O)c2C1] CF3COOH | 538 (M + H)+ | ¹H NMR (400 MHz, DMSO-d₆) δ 8.36 (brs, 2H), 7.62- 7.50 (m, 3H), 7.38-7.27 (m, 2H), 7.20-7.07 (m, 2H), 7.01-6.91 (m, 2H), 4.30 (d, J = 13.1 Hz, 2H), 4.25-4.12 (m, 1H), 4.09-3.97 (m, 2H), 3.78 (t, J = 5.9 Hz, 1H), 3.72 (t, J = 5.8 Hz, 1H), 3.28-3.06 (m, 4H), 2.94- 2.81 (m, 2H), 2.81-2.70 (m, 1H), 2.68-2.59 (m, 1H), 2.45-2.33 (m, 2H), 2.16- 1.84 (m, 6H). | 2-cyclobutyl- sulfanyl-6- [3-[3-(4- fluorophenoxy) propylamino] propanoyl]- 3-phenyl- 7,8-dihydro- 5H-pyrido [4,3-d]pyrimidin- 4-one trifluoroacetate |
| Ex. 48 | 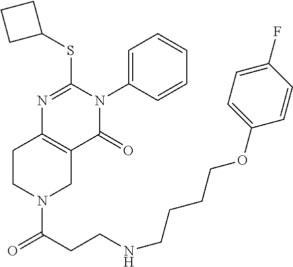 [SMILES: O=C(CCNCCCCOc1ccc(F)cc1)N1CCc2nc(SC3CCC3)n(-c3ccccc3)c(=O)c2C1] CF3COOH | 552 (M + H)+ | ¹H NMR (400 MHz, DMSO-d₆) δ 8.30 (brs, 2H), 7.61- 7.50 (m, 3H), 7.36 (m, 2H), 7.17-7.06 (m, 2H), 6.99- 6.90 (m, 2H), 4.30 (d, J = 13.3 Hz, 2H), 4.25-4.12 (m, 1H), 4.03-3.92 (m, 2H), 3.78 (t, J = 5.9 Hz, 1H), 3.72 (t, J = 5.8 Hz, 1H), 3.25-3.10 (m, 2H), 3.09- 2.96 (m, 2H), 2.92-2.79 (m, 2H), 2.79-2.71 (m, 1H), 2.10-2.58 (m, 1H), 2.45- 2.34 (m, 2H), 2.07-1.87 (m, 4H), 1.82-1.69 (m, 4H). | 2-cyclobutyl- sulfanyl-6- [3-[4-(4- fluorophenoxy) butylamino] propanoyl]- 3-phenyl- 7,8-dihydro- 5H-pyrido [4,3-d]pyrimidin- 4-one trifluoroacetate |
| Ex. 49 | 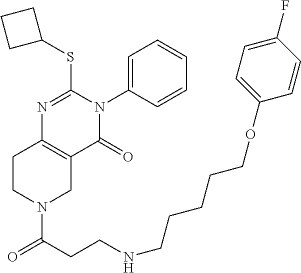 [SMILES: O=C(CCNCCCCCOc1ccc(F)cc1)N1CCc2nc(SC3CCC3)n(-c3ccccc3)c(=O)c2C1] CF3COOH | 566 (M + H)+ | ¹H NMR (400 MHz, DMSO-d₆) δ 8.28 (brs, 2H), 7.62- 7.50 (m, 3H), 7.38-7.26 (m, 2H), 7.18- 7.03 (m, 2H), 6.99-6.87 (m, 2H), 4.30 (d, J = 13.4 Hz, 2H), 4.26-4.11 (m, 1H), 4.01-3.89 (m, 2H), 3.78 (t, J = 5.8 Hz, 1H), 3.71 (t, J = 5.8 Hz, 1H), 3.27-3.11 (m, 3H), 3.05- 2.90 (m, 2H), 2.90-2.79 (m, 2H), 2.79-2.71 (m, 1H), 2.71-2.58 (m, 1H), 2.46- 2.32 (m, 2H), 2.07-1.84 (m, 4H), 1.81-1.57 (m, 4H), 1.55-1.37 (m, 2H). | 2-cyclobutyl- sulfanyl-6- [3-[5-(4- fluorophenoxy) pentylamino] propanoyl]- 3-phenyl- 7,8-dihydro- 5H-pyrido [4,3-d]pyrimidin- 4-one trifluoroacetate |
| Ex. 50 | 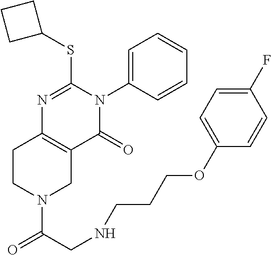 [SMILES: O=C(CNCCCOc1ccc(F)cc1)N1CCc2nc(SC3CCC3)n(-c3ccccc3)c(=O)c2C1] CF3COOH | 523 (M + H)+ | ¹H NMR (400 MHz, DMSO-d₆) δ 8.87 (s, 2H), 7.61-7.51 (m, 3H), 7.37-7.28 (m, 2H), 7.20-7.08 (m, 2H), 7.00- 6.89 (m, 2H), 4.34 (s, 1H), 4.31-4.12 (m, 4H), 4.04 (dt, J = 8.6, 5.9 Hz, 2H), 3.82 (t, J = 5.8 Hz, 1H), 3.67 (t, J = 5.8 Hz, 3H), 3.04-3.18 (m, 2H), 2.78 (t, J = 5.8 Hz, 1H), 2.65 (d, J = 6.0 Hz, 1H), 2.44-2.30 (m, 2H), 2.11 (td, J = 8.6, 5.5 Hz, 2H), 2.04- 1.85 (m, 4H). | 2-cyclobutyl- sulfanyl-6- [2-[3-(4- fluorophenoxy) propylamino] acetyl]- 3-phenyl-7,8- dihydro-5H- pyrido[4,3-d] pyrimidin-4-one trifluoroacetate |
| Ex. 51 | 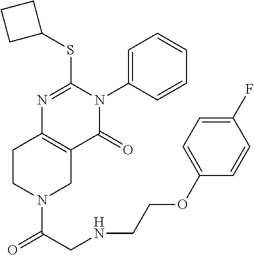 [SMILES: O=C(CNCCOc1ccc(F)cc1)N1CCc2nc(SC3CCC3)n(-c3ccccc3)c(=O)c2C1] CF3COOH | 509 (M + H)+ | ¹H NMR (400 MHz, DMSO-d₆) δ 9.12-9.00 (br, 2H), 7.61- 7.51 (m, 3H), 7.36-7.28 (m. 2H), 7.21-7.10 (m, 2H), 7.05-6.94 (m, 2H), 4.37- 4.13 (m, 7H), 3.81 (t, J = 5.8 Hz, 1H), 3.67 (t, J = 5.8 Hz, 1H), 3.52-3.36 (m, 2H), 2.78 (t, J = 5.8 Hz, 1H), 2.70-2.60 (m, 1H), 2.45- 2.34 (m, 2H), 2.05-1.87 (m, 4H). | 2-cyclobutyl- sulfanyl-6- [2-[2-(4- fluorophenoxy) ethylamino] acetyl]-3- phenyl-7,8- dihydro-5H- pyrido[4,3- d]pyrimidin- 4-one trifluoroacetate |
| Ex. 52 | 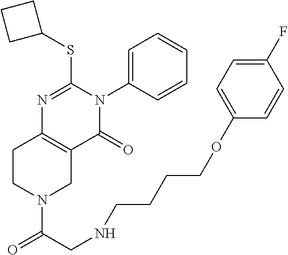 [SMILES: O=C(CNCCCCOc1ccc(F)cc1)N1CCc2nc(SC3CCC3)n(-c3ccccc3)c(=O)c2C1] CF3COOH | 537 (M + H)+ | ¹H NMR (400 MHz, DMSO-d₆) δ 8.83 (s, 2H), 7.61-7.51 (m, 3H), 7.37-7.26 (m, 2H), 7.17-7.07 (m, 2H), 6.99- 6.89 (m, 2H), 4.33 (s, 1H), 4.28-4.12 (m, 4H), 3.96 (q, J = 5.9 Hz, 2H), 3.82 (t, J = 5.8 Hz, 2H), 3.66 (t, J = 5.8 Hz, 4H), 3.07-2.91 (m, 2H), 2.78 (t, J = 5.9 Hz, 1H), 2.65 (d, J = 5.9 Hz, 1H), 2.29-2.44 (m, 2H), 2.04- 1.86 (m, 4H), 1.87-1.67 (m, 4H). | 2-cyclobutyl- sulfanyl-6- [2-[4-(4- fluorophenoxy) butylamino] acetyl]-3- phenyl-7,8- dihydro-5H- pyrido[4,3-d] pyrimidin-4- one trifluoroacetate |
| Ex. 53 | 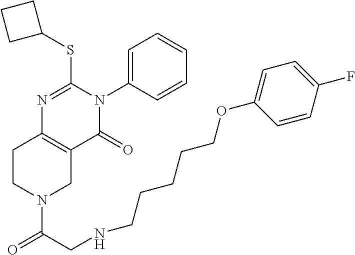 [SMILES: O=C(CNCCCCCOc1ccc(F)cc1)N1CCc2nc(SC3CCC3)n(-c3ccccc3)c(=O)c2C1] CF3COOH | 552 (M + H)+ | ¹H NMR (400 MHz, DMSO-d₆) δ 8.98-8.66 (m, 2H), 7.68- 7.49 (m, 3H), 7.40-7.27 (m, 2H), 7.19-7.05 (m, 2H), 7.00-6.86 (m, 2H), 4.29 (d, J = 33.9 Hz, 2H), 4.23-4.15 (m, 2H), 4.01-3.88 (m, 3H), 3.87-3.78 (m, 1H), 3.73- 3.62 (m, 1H), 3.00-2.85 (m, 2H), 2.84-2.74 (m, 1H), 2.71-2.61 (m, 1H), 2.46- 2.30 (m, 2H), 2.07-1.87 (m, 4H), 1.82-1.59 (m, 5H), 1.53-1.33 (m, 3H). | 2-cyclobutyl- sulfanyl-6- [2-[5-(4- fluorophenoxy) pentylamino] acetyl]-3- phenyl-7,8- dihydro-5H-pyrido [4,3-d]pyrimidin- 4-one trifluoroacetate |
| Ex. 54 | 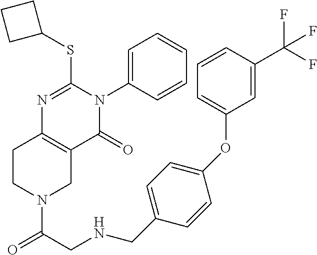 [SMILES: O=C(CNCc1ccc(Oc2cccc(C(F)(F)F)c2)cc1)N1CCc2nc(SC3CCC3)n(-c3ccccc3)c(=O)c2C1] CF3COOH | 621 (M + H)+ | ¹H NMR (400 MHz, DMSO-d₆) δ 9.45-9.21 (br, 2H), 7.70- 7.47 (m, 7H), 7.40-7.29 (m, 4H), 7.21-7.10 (m, 2H), 4.43-4.08 (m, 7H), 3.82 (t, J = 5.8 Hz, 1H), 3.65 (t, J = 5.8 Hz, 1H), 2.77 (t, J = 5.7 Hz, 1H), 2.66 (t, J = 5.9 Hz, 1H), 2.46-2.31 (m, 2H), 2.06-1.84 (m, 4H). | 2-cyclobutyl- sulfanyl-3- phenyl-6-[2- [[4-[3-(trifluoro- methyl)phenoxy] phenyl]methylamino] acetyl]-7,8- dihydero-5H- pyrido[4,3-d] pyrimidin-4- one trifluoroacetate |
| Ex. 55 | 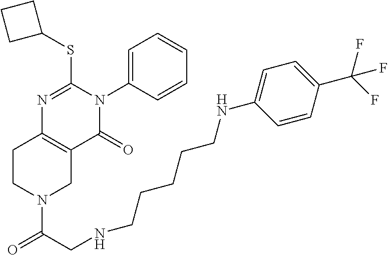 [SMILES: O=C(CNCCCCCNc1ccc(C(F)(F)F)cc1)N1CCc2nc(SC3CCC3)n(-c3ccccc3)c(=O)c2C1] CF3COOH | 600 (M + H)+ | ¹H NMR (400 MHz, DMSO-d₆) δ 8.88-8.71 (br, 2H), 7.63- 7.51 (m, 3H), 7.41-7.26 (m, 4H), 6.64 (dd, J = 8.6, 4.5 Hz, 2H), 6.46-6.20 (br, 1H), 4.38-4.11 (m, 5H), 3.81 (t, J = 5.8 Hz, 1H), 3.66 (t, J = 5.9 Hz, 1H), 3.10-3.00 (m, 2H), 2.96-2.86 (t, J = 5.7 Hz, 1H), 2.70-2.61 (m, 1H), 2.45-2.34 (m, 2H), 2.03-1.88 (m, 4H), 1.67 (tt, J = 7.8, 7.0 Hz, 2H), 1.55 (tt, J = 7.8, 7.5 Hz, 2H), 1.40 (tt, J = 7.8, 7.8 Hz 2H). | 2-cyclobutyl- sulfanyl-3- phenyl-6-[2- [5-[4-(trifluoro- methyl)anilino] pentylamino] acetyl]-7,8- dihydro-5H- pyrido[4,3-d] pyrimidin-4-one trifluoroacetate |
| Ex. 56 | 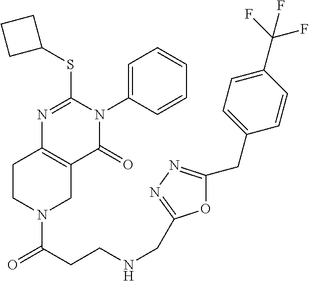 [SMILES: O=C(CCNCc1nnc(Cc2ccc(C(F)(F)F)cc2)o1)N1CCc2nc(SC3CCC3)n(-c3ccccc3)c(=O)c2C1] CF3COOH | 625 (M + H)+ | ¹H NMR (400 MHz, DMSO-d₆) δ 9.28 (s, 2H), 7.80-7.70 (m, 2H), 7.63-7.51 (m, 5H), 7.35-7.27 (m, 2H), 4.60 (s, 2H), .48 (d, J = 2.5 Hz, 2H), 4.29 (d, J = 14.8 Hz, 2H), 2.89 (q, J = 6.4 Hz, 2H), 2.81-2.71 (m, 1H), 2.69- 2.63 (m, 2H), 2.45-2.30 (m, 3H), 2.03-1.85 (m, 4H). | 2-cyclobutyl- sulfanyl[-3- phenyl-6-[3- [5-[[4-(tri- fluoromethyl) phenyl]methyl]- 1,3,4-oxadiazol- 2-yl]methylamino] propanoyl]-7,8- dihydro-5H- pyrido[4,3-d] pyrimidin-4- one trifluoroacetate |
| Ex. 57 | 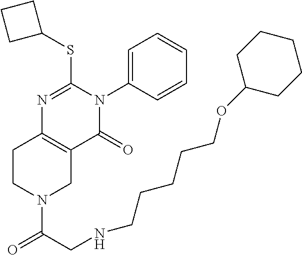 [SMILES: O=C(CNCCCCCOC1CCCCC1)N1CCc2nc(SC3CCC3)n(-c3ccccc3)c(=O)c2C1] CF3COOH | 540 (M + H)+ | ¹H NMR (400 MHz, DMSO-d₆) δ 8.85 (brs, 2H), 7.62- 7.50 (m, 3H), 7.39-7.26 (m, 2H), 4.40-4.10 (m, 5H), 3.81 (t, J = 5.85 Hz, 1H), 3.66 (t, J = 5.81 Hz, 1H), 3.39-3.34 (m, 2H), 3.25- 3.14 (m, 1H), 2.98-2.84 (m, 2H), 2.78 (t, J = 5.70 Hz, 1H), 2.65 (t, J = 5.70 Hz, 1H), 2.46-2.32 (m, 2H), 2.04- 1.87 (m, 4H), 1.86-1.72 (m, 2H), 1.72-1.55 (m, 4H), 1.55-1.41 (m, 3H), 1.41- 1.27 (m, 2H), 1.27-1.09 (m, 5H). | 2-cyclobutyl- sulfanyl-6- [2-[5-(cyclo- hexoxy)pentyl- amino]acetyl]-3- phenyl-7,8- dihydro-5H- pyrido[4,3-d] pyrimidin-4- one trifluoroacetate |
| Ex. 58 | 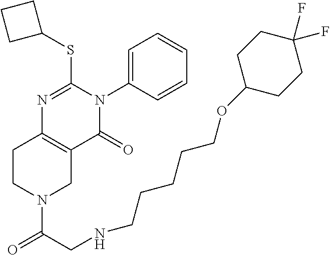 [SMILES: O=C(CNCCCCCOC1CCC(F)(F)CC1)N1CCc2nc(SC3CCC3)n(-c3ccccc3)c(=O)c2C1] CF3COOH | 576 (M + H)+ | ¹H NMR (400 MHz, DMSO-d₆) δ 8.87 (brs, 2H), 7.64- 7.51 (m, 3H), 7.38-7.27 (m, 2H), 4.29 (d, J = 33.6 Hz, 2H), 4.23-4.13 (m, 3H), 3.81 (t, J = 5.9 Hz, 1H), 3.66 (t, J = 5.8 Hz, 1H), 3.51-3.43 (m, 1H), 3.38-3.25 (m, 2H), 2.99-2.84 (m, 2H), 2.78 (t, J = 5.7 Hz, 1H), 2.65 (t, J = 5.9 Hz, 1H), 2.45-2.33 (m, 2H), 2.05-1.58 (m, 14H), 1.57-1.45 (m, 2H), 1.41- 1.28 (m, 2H). | 2-cyclobutyl- sulfanyl-6- [2-[5-(4,4-di- fluorocyclo- hexoxy)pentyl- amino]acetyl]- 3-phenyl-7,8- dihydro-5H- pyrido[4,3-d] pyrimidin-4- one trifluoroacetate |
| Ex. 59 | 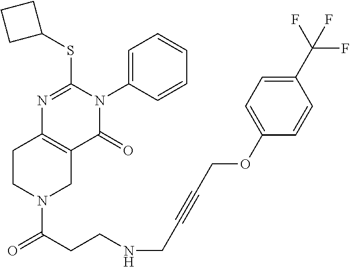 [SMILES: O=C(CCNCC#CCOc1ccc(C(F)(F)F)cc1)N1CCc2nc(SC3CCC3)n(-c3ccccc3)c(=O)c2C1] CF3COOH | 597 (M + H)+ | ¹H NMR (400 MHz, DMSO-d₆) δ 8.93 (s, 2H), 7.73-7.65 (m, 2H), 7.59-7.52 (m, 3H), 7.37-7.27 (m, 2H), 7.24- 7.14 (m, 2H), 5.04 (dt, J = 3.5, 1.8 Hz, 2H), 4.29 (d, J = 18.5 Hz, 2H), 4.23-4.13 (m, 1H), 4.05 (s, 2H), 3.77 (t, J = 5.8 Hz, 1H), 3.69 (t, J = 5.8 Hz, 2H), 3.21 (s, 2H), 2.84 (q, J = 6.7 Hz, 2H), 2.74 (t, J = 5.8 Hz, 1H), 2.64 (t, J = 5.8 Hz, 1H), 2.45-2.30 (m, 2H), 2.07- 1.85 (m, 4H). | 2-cyclobutyl- sulfanyl-3- phenyl-6-[3- [4-[4-(tri- fluoromethyl) phenoxy]but-2- ynylamino] propanoyl]-7,8- dihydro-5H- pyrido[4,3-d] pyrimidin-4- one trifluoroacetate |
| Ex. 60 | 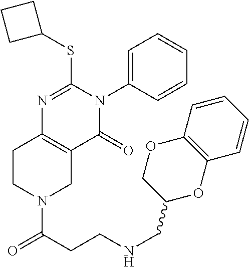 [SMILES: O=C(CCNCC1COc2ccccc2O1)N1CCc2nc(SC3CCC3)n(-c3ccccc3)c(=O)c2C1] CF3COOH | 533 (M + H)+ | ¹H NMR (400 MHz, DMSO-d₆) δ 8.68 (br, 2H), 7.61- 7.51 (m, 3H), 7.36-7.28 (m, 2H), 6.97-6.84 (m, 4H), 4.65-4.56 (m, 1H), 4.38- 4.26 (m, 3H), 4.24-4.14 (m, 1H), 4.10-3.99 (m, 1H), 3.79 (t, J = 5.8 Hz, 1H), 3.72 (t, J = 5.7 Hz, 1H), 3.41-3.36 (m, 1H), 3.33- 3.24 (m, 3H), 2.95-2.85 (m, 2H), 2.75 (t, J = 5.9 Hz, 1H), 2.70-2.60 (m, 1H), 2.44- 2.34 (m, 2H), 2.02-1.88 (m, 4H). | 2-cyclobutyl- sulfanyl-6- [3-(2,3-dihydro- 1,4-benzo- dioxin-3- ylmethylamino) propanoyl]-3- phenyl-7,8- dihydro-5H- pyrido[4,3-d] pyrimidin-4- one trifluoroacetate |

### [Pharmacological Test]

The compounds of Examples 1 to 24 of the present invention were investigated for their pharmacological action.

### (1) Preparation of Recombinant Human MGAT2 Enzyme>

The human MGAT2 gene described in Am. J. Physiol. Endocrinol. Metab. 285: E927-E937, 2003 was expressed using Jump-In™ CHO-K1 Kit (Life technologies) as described in the attached manual. The cells were suspended in a buffer containing Tris-hydrochloric acid (pH 7.4) at a final concentration of 1 mM, 1 mM ethylenediaminetetraacetic acid, 1 mM dithiothreitol, 250 mM sucrose, and complete protease inhibitor cocktail (Roche applied Science), and then disrupted using a Teflon homogenizer (AS ONE Corporation), followed by centrifugation at 130 g for 10 minutes. The supernatant was further centrifuged at 100,000 g for 1 hour to obtain a pellet as microsomes containing the recombinant enzyme, and a solution obtained by suspending this pellet at a protein concentration of about 800 to 1900 μg/ml was used as an MGAT recombinant enzyme solution.

### (2) Test for Human MGAT2 Activity Inhibitory Action

To a buffer containing 50 mM Tris-hydrochloric acid (pH 7.4), 6 mM magnesium chloride, and 2 mg/mL bovine serum albumin, a test compound was added, and the final concentration of dimethyl sulfoxide was adjusted to 0.3%. Moreover, 2-monooleoyl glycerol, oleoyl CoA, 1-¹⁴C-oleoyl CoA (Perkin Elmer), and orlistat (SIGMA) were added at final concentrations of 50 μM, 50 μM, 2.2 μM, and 8.3 μM, respectively, followed by incubation at 37° C. for 10 minutes. After 10 minutes, the recombinant human MGAT2 enzyme solution was added at a final concentration of 0.67 μg/mL, and the reaction was started, with the amount of the reaction solution being 150 μl. The reaction was carried out at 37° C. for 30 minutes.

After 30 minutes, the reaction was stopped by adding 300 μl of isopropanol:heptane:water=(80:20:2) to the reaction liquid. Further, 200 μl of heptane and 100 μl of water were added, followed by stirring. Then, 100 μl of the upper layer was transferred to a 96-well plate (Corning), and mixed with 100 μl of liquid scintillator (LumaSafe Plus, Lumac). Then, the ¹⁴C count was quantitatively determined using TopCount NXT™ microplate scintillation luminescence counter (Perkin Elmer).

The ¹⁴C count in a case where no test compound was added was defined as A, the ¹⁴C count in a case where 10 μM of the compound (Comparative Example 1) of Example No. 8 of WO2008/038768 was added was defined as B, and the ¹⁴C count in the case where the test compound was added was taken as C. For this case, {1−(C−B)/(A−B)}×100(%) was calculated as the human MGAT2 activity inhibition ratio of the test compound (shown in the column named vs Comp. Ex. 1 in the following Table). A greater value of the “vs Comparative Example 1” means that the MGAT2 activity inhibitory action of the compound of Example was higher as compared to the MGAT2 activity inhibitory action of the compound of Comparative Example 1 (i.e., the human MGAT2 activity inhibition ratio was higher). In addition, for each of Examples 1 to 24 and Comparative Example 1, the inhibition ratios at various concentrations were calculated, and the 50% inhibition concentration (IC₅₀ value) was found by a four-parameter logistic regression analysis using XLFit (IDBS). Since this evaluation test was conducted for two separate groups, the test results of the two groups are shown separately in Table (First Test) and Table 4 (Second Test).

TABLE 3

| Compound No. | IC50 (nM) | vs Comp. Ex. 1 |
|---|---|---|
| Example 1 | 4.1 | 54 |
| Example 2 | 1.3 | 171 |
| Example 3 | 3.7 | 60 |
| Example 4 | 2.9 | 77 |
| Example 5 | 0.64 | 349 |
| Example 6 | 1.6 | 144 |
| Example 7 | 2.3 | 75 |
| Example 8 | 3.5 | 63 |
| Example 9 | 3.9 | 57 |
| Example 10 | 0.82 | 273 |
| Comparative Example 1 | 223.0 | — |
| (Compound of Example | | |
| 8 in WO2008/038768)* | | |

*The chemical structural formula of Comparative Example 1 is shown below (hereinafter, the same).

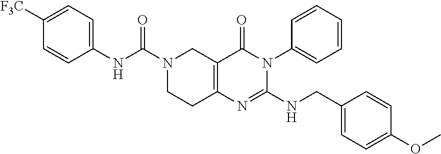 [SMILES: COc1ccc(CNc2nc3c(c(=O)n2-c2ccccc2)CN(C(=O)Nc2ccc(C(F)(F)F)cc2)CC3)cc1]

TABLE 4

| Compound No. | IC50 (nM) | vs Comp. Ex. 1 |
|---|---|---|
| Example 11 | 1.8 | 166 |
| Example 12 | 1.4 | 216 |
| Example 13 | 6.9 | 44 |
| Example 14 | 4.7 | 65 |
| Example 15 | 2.4 | 129 |
| Example 16 | 1.8 | 173 |
| Example 17 | 2.1 | 148 |
| Example 18 | 2.3 | 134 |
| Example 19 | 0.9 | 352 |
| Example 20 | 1.0 | 300 |
| Example 21 | 1.2 | 246 |
| Example 22 | 3.8 | 79 |
| Example 23 | 1.6 | 193 |
| Example 24 | 1.4 | 221 |
| Comparative Example 1 | 303.0 | — |
| (Compound of Example 8 | | |
| of WO2008/038768) | | |

As can be seen from Tables 3 and 4, the compounds of the present invention achieved extremely higher MGAT2 inhibitory activity than the compound of Comparative Example 1, which is a conventional compound, because of their structure, especially, because of the presence of the structure of “—C(═O)—R₁—NH—R₂—X—,” which is absent in the conventional compound. Accordingly, it is conceivable that the compounds of the present invention are better as MGAT2 inhibitors than conventional compounds, have fat absorption inhibitory action, and effectively act on lipid metabolism disorders and adiposity.

Next, the compounds of Examples 25 to 60 of the present invention were also investigated for their pharmacological action by using the same method as in Examples 1 to 24. Table 5 shows the results. Note that, a comparative evaluation test against the compound of Comparative Example 1 was conducted for each of the compounds of Examples (in every measurement of the inhibitory activity of the compounds of Examples 25 to 60, the inhibitory activity of the compound of Comparative Example 1 was measured). The 50% inhibition concentrations of Comparative Example 1 in the measurement of the inhibitory activities of the compounds of Examples 25 to 60 were shown in Table 5 as “IC50 (nM) of Comparative Example 1.”

TABLE 5

| | | vs Comp. | IC50 (nM) of |
|---|---|---|---|
| Compound No. | IC50 (nM) | Ex. 1 | Comp. Ex. 1 |
| Example 25 | 2.2 | 111 | 244 |
| Example 26 | 1.0 | 196 | 200 |
| Example 27 | 1.0 | 194 | 200 |
| Example 28 | 1.7 | 125 | 210 |
| Example 29 | 1.5 | 138 | 210 |
| Example 30 | 3.0 | 69 | 210 |
| Example 31 | 1.6 | 130 | 210 |
| Example 32 | 1.3 | 166 | 210 |
| Example 33 | 0.8 | 228 | 170 |
| Example 34 | 3.2 | 47 | 150 |
| Example 35 | 1.1 | 130 | 150 |
| Example 36 | 0.7 | 205 | 140 |
| Example 37 | 0.5 | 273 | 140 |
| Example 38 | 1.4 | 211 | 295 |
| Example 39 | 0.4 | 320 | 137 |
| Example 40 | 0.7 | 245 | 170 |
| Example 41 | 1.1 | 151 | 170 |
| Example 42 | 1.2 | 148 | 170 |
| Example 43 | 2.5 | 72 | 180 |
| Example 44 | 1.8 | 135 | 250 |
| Example 45 | 1.3 | 226 | 300 |
| Example 46 | <1.0 | >184 | 180 |
| Example 47 | 1.1 | 166 | 180 |
| Example 48 | <1.0 | >184 | 180 |
| Example 49 | <1.0 | >184 | 180 |
| Example 50 | 3.7 | 73 | 270 |
| Example 51 | 4.2 | 64 | 270 |
| Example 52 | 1.7 | 158 | 270 |
| Example 53 | 2.0 | 133 | 270 |
| Example 54 | 2.6 | 91 | 240 |
| Example 55 | <1.0 | >207 | 210 |
| Example 56 | 5.8 | 35 | 210 |
| Example 57 | 2.6 | 81 | 210 |
| Example 58 | 3.3 | 63 | 210 |
| Example 59 | 2.2 | 117 | 260 |
| Example 60 | 1.1 | 140 | 160 |

As can be seen from Table 5, the compounds of the present invention achieved extremely higher MGAT2 inhibitory activity than the compound of Comparative Example 1 which is a conventional compound, because of their structure, especially because of the presence of the structure of “—C(═O)—R₁—NH—R₂—X—,” which is absent in the conventional compound. Accordingly, it is conceivable that the compounds of the present invention are better as MGAT2 inhibitors than conventional compounds, have fat absorption inhibitory action, and effectively act on lipid metabolism disorders and adiposity.

### Test for Anti-Obesity Action

The anti-obesity action of the compound of Example 5 was examined in C57BL/6J mice fed on high-fat diet (Research Diets D12492). Thirteen-week old male C57BL/6J-DIO mice (CHARLES RIVER LABORATORIES JAPAN, INC.) were purchased, and fed in animal facilities under a 12-hour light/dark cycle for 13 weeks on the diet of D12492. From six days before the administration of the compound, the animals were habituated by inserting a tube for forced oral administration into the stomach once per day. One day before the administration of the compound, the mice were randomized on the basis of the body weight, and divided into groups (n=8). From Day 1 to Day 22, forced oral administration of the compound or a medium (0.5% methyl cellulose) was conducted once per day. The body weight was measured every day, and the body fat weight and the fat-free weight were measured by using a quantitative NMR apparatus (EcoMRI, Hitachi Aloka Medical, Ltd.) on Day 21.

The percent body weight change (Day 23, Table 6), the body fat weight (Day 21, Table 7), and the fat-free weight (Day 21, Table 8) were each expressed in terms of the difference between the average value of the compound administration group and the average value of the medium administration group, and the difference between the groups was subjected to Dunnett's multiple comparison test (significance level p=0.05). Tables 6 to 8 show the results. Note that, in Tables 6 to 8, “n.s.” means a non-significant difference.

TABLE 6
Table 6: Percent Body Weight Change

| | Average value | | Dunnett's |
|---|---|---|---|
| | (%, difference | | test (comparison |
| Amount of | from medium | | with medium |
| compound | administration | Standard | administration |
| administered | group) | error | group) |
| 10 mg/kg | −4.15 | 0.63 | n.s. |
| 30 mg/kg | −9.56 | 2.84 | p < 0.01 |

TABLE 7
Table 7: Body Fat Weight

| | Average value | | Dunnett's |
|---|---|---|---|
| | (g, difference | | test (comparison |
| Amount of | from medium | | with medium |
| compound | administration | Standard | administration |
| administered | group) | error | group) |
| 10 mg/kg | −2.22 | 0.39 | n.s. |
| 30 mg/kg | −4.26 | 1.09 | p < 0.001 |

TABLE 8
Table 8: Fat-Free Weight

| | Average value | | Dunnett's |
|---|---|---|---|
| | (g, difference | | test (comparison |
| Amount of | from medium | | with medium |
| compound | administration | Standard | administration |
| administered | group) | error | group) |
| 10 mg/kg | −0.34 | 0.43 | n.s. |
| 30 mg/kg | −0.98 | 0.50 | n.s. |

From the results in Tables 6 and 7, it is apparent that the percent body weight change and the body fat weight were significantly lowered in the group to which the compound of Example 5 was administered at 30 mg/kg. In addition, from the results in Table 8, it was shown that the decrease of the fat-free body weight caused by the compound of Example 5 did not have a statistically significant difference. In other words, in the diet-induced obesity mouse model, it was shown that the compound of Example 5 brought about the body weight loss effect by decreasing the body fat weight without changing the fat-free weight.

### Test for Lipid Absorption Inhibitory Action

The lipid absorption inhibitory action of the compounds of Examples 5 and 60 and the compound of Comparative Example 1 was examined in an orally lipid-loaded mouse model. In this test, 10 to 11-week old male C57BL/6J mice (CLEA Japan, Inc.) were used. After the mice were fasted for 17 hours from the preceding day, blood was collected from the fundus, and further the mice were subjected to forced oral administration of one of the compounds or the medium (0.5% methyl cellulose). Thirty minutes after the administration, tyloxapol (500 mg/kg) was administered into the tail vein, and corn oil (200 μl) was orally administered (n=5-6). Blood was collected via the tail vein 2, 4, and 6 hours after the administration of corn oil, and plasma was obtained by centrifuging the blood. The neutral fat concentration in the plasma was measured by using Triglyceride E-Test Wako (Wako Pure Chemical Industries, Ltd.). The neutral fat concentration in the plasma before the administration of the compound was taken as the value at 0 hours, and the area under the curve (AUC) of the neutral fat concentration in the plasma up to 6 hours after the administration of corn oil was calculated. From the average value of the AUC of each administration group, the lipid absorption inhibition ratio was obtained by using the following formula.

Lipid absorption inhibition ratio (%)=(Average value of AUC of medium administration group-Average value of AUC of compound administration group)/Average value of AUC of medium administration group×100

Table 9 shows the results.

TABLE 9
Table 9: Lipid Absorption Inhibition Ratio in Orally
Lipid-Loading Test

| | | | Lipid absorption |
|---|---|---|---|
| | | Administered | inhibition ratio |
| | Compound | amount | (%) |
| Test 1 | Comp. Ex. 1 | 30 mg/kg | 34 |
| | Comp. Ex. 1 | 100 mg/kg | 44 |
| Test 2 | Example 5 | 30 mg/kg | 45 |
| | Comp. Ex. 1 | 100 mg/kg | 41 |
| Test 3 | Example 5 | 30 mg/kg | 42 |
| | Example 60 | 30 mg/kg | 42 |

From the results in Table 9, it was found that the compound of Example 5 administered in an amount which was ⅓ of that of the compound of Comparative Example 1 exhibited lipid absorption inhibitory action not inferior to that of the compound of Comparative Example 1. In addition, it has been found that the compound of Example 60 has excellent lipid absorption inhibitory action comparable to that of the compound of Example 5.

### INDUSTRIAL APPLICABILITY

The compound represented by general formula (I) of the present invention or a pharmaceutically acceptable salt thereof has extremely higher MGAT2 inhibitory activity than conventional compounds. Accordingly, the compound represented by general formula (I) of the present invention or a pharmaceutically acceptable salt thereof is excellent as an MGAT2 inhibitor, and can be used suitably for inhibiting fat absorption, treating or preventing a lipid metabolism disorder, and treating or preventing adiposity. Accordingly, the compound represented by general formula (I) of the present invention or a pharmaceutically acceptable salt thereof is extremely industrially useful.

1. A compound represented by the following general formula (I) or a pharmaceutically acceptable salt thereof: 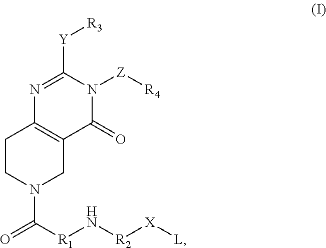
wherein
R₁ represents a linear C₁₋₆ alkylene group which may be optionally substituted by deuterium atoms;
R₂ represents a linear C₁₋₆ alkylene group or acyclic C₃₋₆ alkylene group, which may be optionally substituted by 1 to 6 identical or different substituents selected from deuterium atoms, halogen atoms, C₁₋₃ alkyl groups, C₁₋₃ haloalkyl groups, C₁₋₃ alkylthio groups, nitrile groups, hydroxyl groups, C₁₋₃ alkoxy groups, C₁₋₃ haloalkoxy groups, amino groups, C₁₋₃ alkylamino groups, and C₂₋₆ dialkylamino groups;
X represents a single bond, —O—, —S—, or —NR₅—, where R₅ represents a hydrogen atom, a C₁₋₆ alkyl group, or a C₁₋₆ haloalkyl group;
L represents a C₂₋₆ alkenyl group, a C₂₋₆ alkynyl group, a cycloalkyl group having 3 to 6 carbon atoms, a phenyl group, a naphthyl group, a 3- to 6-membered saturated or unsaturated heterocyclic group, or a 9- or 10-membered saturated or unsaturated heterobicyclic group, which may be optionally substituted by 1 to 5 identical or different substituents selected from deuterium atoms, halogen atoms, hydroxyl groups, amino groups, C₁₋₃ alkyl groups, C₁₋₃ haloalkyl groups, C₁₋₃ alkylthio groups, nitrile groups, C₁₋₃ alkoxy groups, C₁₋₃ haloalkoxy groups, C₁₋₃ alkylamino groups, and C₂₋₆ dialkylamino groups, or which may be optionally monosubstituted by a substituent selected from the following (a) to (e):
(a) a —C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;
(b) a —C₁₋₃ alkylene-O—C₁₋₆ alkyl group which may be optionally substituted by 1 to 5 identical or different substituents selected from deuterium atoms, halogen atoms, C₁₋₃ alkoxy groups, and C₁₋₃ haloalkoxy groups;
(c) a —C₁₋₃ alkylene-O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;
(d) an —O—C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups; and
(e) an —O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;
Y represents a single bond, —S—, —O—, or —NR₆—, where R₆ represents a hydrogen atom, a C₁₋₆ alkyl group, or a C₁₋₆ haloalkyl group;
Z represents a single bond or a C₁₋₆ alkylene group;
R₃ represents a C₃₋₈ alkyl group, a C₃₋₈ cycloalkyl group, or a 3- to 8-membered saturated or unsaturated heterocyclic group, and may optionally have 1 to 7 identical or different substituents selected from the group consisting of deuterium atoms, halogen atoms, C₁₋₃ alkyl groups, C₁₋₃ haloalkoxy groups, and C₁₋₃ haloalkyl groups; and
R₄ represents a C₁₋₆ alkyl group, a phenyl group, a cycloalkyl group having 3 to 8 carbon atoms, or a 3- to 8-membered saturated or unsaturated heterocyclic group, and may optionally have 1 to 7 identical or different substituents selected from the group consisting of deuterium atoms, halogen atoms, C₁₋₃ alkyl groups, C₁₋₃ haloalkyl groups, C₁₋₃ alkoxy groups, and C₁₋₃ haloalkoxy groups.

2. The compound according to claim 1 or a pharmaceutically acceptable salt thereof, wherein
X is a single bond, —O—, —S—, or —NH—.

3. The compound according to claim 1 or a pharmaceutically acceptable salt thereof, wherein
R₁ is a linear C₁₋₃ alkylene group.

4. The compound according to claim 1 or a pharmaceutically acceptable salt thereof, wherein
L is a phenyl group, a naphthyl group, a 5- or 6-membered saturated or unsaturated heterocyclic group, or a 9- or 10-membered saturated or unsaturated heterobicyclic group, which may be optionally substituted by 1 to 5 identical or different substituents selected from deuterium atoms, halogen atoms, hydroxyl groups, amino groups, C₁₋₃ alkyl groups, C₁₋₃ haloalkyl groups, C₁₋₃ alkylthio groups, nitrile groups, C₁₋₃ alkoxy groups, C₁₋₃ haloalkoxy groups, C₁₋₃ alkylamino groups, and C₂₋₆ dialkylamino groups, or which may be optionally monosubstituted by a substituent selected from the following (a) to (e):
(a) a —C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;
(b) a —C₁₋₃ alkylene-O—C₁₋₆ alkyl group which may be optionally substituted by 1 to 5 identical or different substituents selected from deuterium atoms, halogen atoms, C₁₋₃ alkoxy groups, and C₁₋₃ haloalkoxy groups;
(c) a —C₁₋₃ alkylene-O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;
(d) an —O—C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups; and
(e) an —O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups.

5. The compound according to claim 1 or a pharmaceutically acceptable salt thereof, wherein
R₃ is a C₃₋₆ alkyl group or a cycloalkyl group having 3 to 7 carbon atoms.

6. The compound according to claim 1 or a pharmaceutically acceptable salt thereof, wherein
Y is —S—, —O—, or —NH—.

7. The compound according to claim 1 or a pharmaceutically acceptable salt thereof, wherein
Z is a single bond or a C₁₋₃ alkylene group.

8. The compound according to claim 1 or a pharmaceutically acceptable salt thereof, wherein
R₄ is a C₁₋₆ alkyl group, a phenyl group, a cycloalkyl group having 3 to 7 carbon atoms, or a 5- or 6-membered saturated or unsaturated heterocyclic group.

9. The compound according to claim 1 or a pharmaceutically acceptable salt thereof, wherein
the compound is selected from the group consisting of the following compounds:

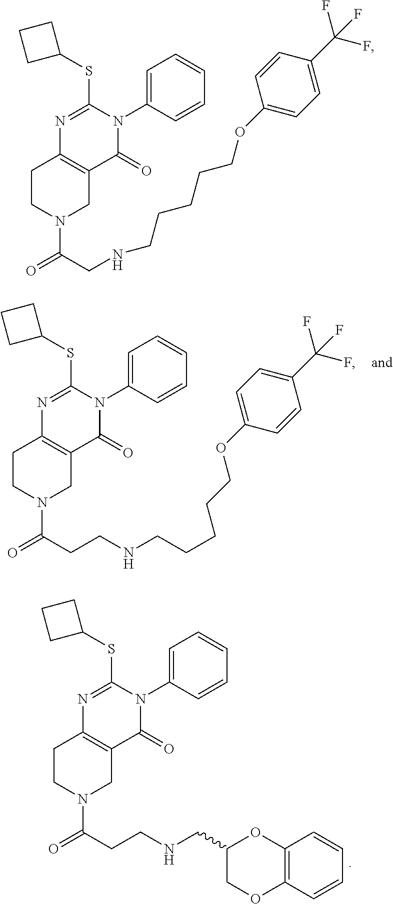

10. A monoacylglycerol acyltransferase (MGAT) inhibitor, comprising the compound according to claim 1 or a pharmaceutically acceptable salt thereof as an active ingredient.

11. A fat absorption inhibitor, comprising the compound according to claim 1 or a pharmaceutically acceptable salt thereof as an active ingredient.

12. An agent for preventing and/or treating adiposity, comprising the compound according to claim 1 or a pharmaceutically acceptable salt thereof as an active ingredient.

13. An agent for preventing and/or treating a lipid metabolism disorder, comprising the compound according to claim 1 or a pharmaceutically acceptable salt thereof as an active ingredient.

14. A pharmaceutical composition, comprising the compound according to claim 1 or a pharmaceutically acceptable salt thereof as an active ingredient.

15. A method for producing a compound represented by the following general formula (I) or a pharmaceutically acceptable salt thereof, comprising the steps of:
(a) adding acryloyl chloride to a compound represented by the following general formula (II) for a reaction therebetween, to obtain a compound represented by the following general formula (III); and
(b) adding a an alkylated amine represented by Formula L-X—R₂—NH₂, where L, X, and R₂ are the same as L, X, and R₂ in general formula (I), respectively, to the compound represented by general formula (III) for a reaction therebetween, to obtain the compound represented by general formula (I): 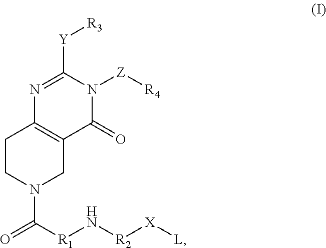
wherein
R₁ represents a linear ethylene group which may be optionally substituted by deuterium atoms;
R₂ represents a linear C₁₋₆ alkylene group or acyclic C₃₋₆ alkylene group, which may be optionally substituted by 1 to 6 identical or different substituents selected from deuterium atoms, halogen atoms, C₁₋₃ alkyl groups, C₁₋₃ haloalkyl groups, C₁₋₃ alkylthio groups, nitrile groups, hydroxyl groups, C₁₋₃ alkoxy groups, C₁₋₃ haloalkoxy groups, amino groups, C₁₋₃ alkylamino groups, and C₂₋₆ dialkylamino groups;
X represents a single bond, —O—, —S—, or —NR₅—, where R₅ represents a hydrogen atom, a C₁₋₆ alkyl group, or a C₁₋₆ haloalkyl group;
L represents a C₂₋₆ alkenyl group, a C₂₋₆ alkynyl group, a cycloalkyl group having 3 to 6 carbon atoms, a phenyl group, a naphthyl group, a 3- to 6-membered saturated or unsaturated heterocyclic group, or a 9- or 10-membered saturated or unsaturated heterobicyclic group, which may be optionally substituted by 1 to 5 identical or different substituents selected from deuterium atoms, halogen atoms, hydroxyl groups, amino groups, C₁₋₃ alkyl groups, C₁₋₃ haloalkyl groups, C₁₋₃ alkylthio groups, nitrile groups, C₁₋₃ alkoxy groups, C₁₋₃ haloalkoxy groups, C₁₋₃ alkylamino groups, and C₂₋₆ dialkylamino groups, or which may be optionally monosubstituted by a substituent selected from the following (a) to (e):
(a) a —C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;
(b) a —C₁₋₃ alkylene-O—C₁₋₆ alkyl group which may be optionally substituted by 1 to 5 identical or different substituents selected from deuterium atoms, halogen atoms, C₁₋₃ alkoxy groups, and C₁₋₃ haloalkoxy groups;
(c) a —C₁₋₃ alkylene-O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;
(d) an —O—C₁₋₃ alkylene-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups; and
(e) an —O-phenyl group which may be optionally substituted by 1 to 5 identical or different substituents selected from deuterium atoms, halogen atoms, C₁₋₃ alkyl groups, C₁₋₃ haloalkyl groups, C₁₋₃ alkylthio groups, nitrile groups, C₁₋₃ alkoxy groups, and C₁₋₃ haloalkoxy groups;
Y represents a single bond, —S—, —O—, or —NR₆—, where R₆ represents a hydrogen atom, a C₁₋₆ alkyl group, or a C₁₋₆ haloalkyl group;
Z represents a single bond or a C₁₋₆ alkylene group;
R₃ represents a C₃₋₈ alkyl group, a C₃₋₈ cycloalkyl group, or a 3- to 8-membered saturated or unsaturated heterocyclic group, and may optionally have 1 to 7 identical or different substituents selected from the group consisting of deuterium atoms, halogen atoms, C₁₋₃ alkyl groups, C₁₋₃ haloalkoxy groups, and C₁₋₃ haloalkyl groups; and
R₄ represents a C₁₋₆ alkyl group, a phenyl group, a cycloalkyl group having 3 to 8 carbon atoms, or a 3- to 8-membered saturated or unsaturated heterocyclic group, and may optionally have 1 to 7 identical or different substituents selected from the group consisting of deuterium atoms, halogen atoms, C₁₋₃ alkyl groups, C₁₋₃ haloalkyl groups, C₁₋₃ alkoxy groups, and C₁₋₃ haloalkoxy groups; 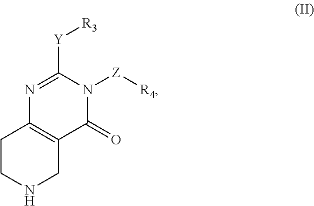
wherein
Y, Z, R₃, and R₄ are the same as Y, Z, R₃, and R₄ in general formula (I), respectively; and 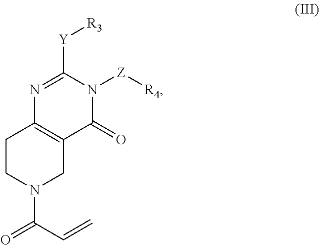
wherein
Y, Z, R₃, and R₄ are the same as Y, Z, R₃, and R₄ in general formula (I), respectively.
